(19) United States
(12) Patent Application Publication
(10) Pub. No.: US 2016/0115527 A1
(43) Pub. Date: Apr. 28, 2016

(54) COMPOSITIONS, KITS, AND RELATED METHODS FOR DETECTING AND/OR MONITORING SHIGA TOXIN PRODUCING ESCHERICHIA COLI

(71) Applicant: Roka Bioscience, Inc., Warren, NJ (US)

(72) Inventors: Michael McClellan BECKER, San Diego, CA (US); Bettina Groschel, San Diego, CA (US); Kristin LIVEZEY, Encinitas, CA (US)

(21) Appl. No.: 14/984,750

(22) Filed: Dec. 30, 2015

Related U.S. Application Data
(63) Continuation of application No. PCT/US2014/045485, filed on Jul. 3, 2014.
Application No. PCT/US2014/045485 is a continuation-in-part of application No. 14/369,888, filed on Jun. 30, 2014.
(60) Provisional application No. 61/842,924, filed on Jul. 3, 2013.
(60) Provisional application No. 61/582,153, filed on Dec. 30, 2011.
(60) Provisional application No. 61/646,180, filed on May 11, 2012.

(51) Int. Cl. C12Q 1/68; B65B 25/06; G01N 33/569

(52) U.S. Cl. CPC C12Q 1/689; G01N 33/56916; A23L 1/31; B65B 25/065; C12Q 2600/158; G01N 2333/245; A23V 2002/00

(57) ABSTRACT

The invention relates to a method for testing a sample for the presence of a pathogenic Escherichia coli, the method including detecting the presence of (i) ecf and (ii) wzx and/or stx in the sample, wherein detection of (i) ecf and (ii) wzx and/or stx in the sample is taken as an indication that the sample includes pathogenic E. coli.

### CROSS-REFERENCE TO RELATED APPLICATIONS

This application claims benefit of U.S. Provisional Application No. 61/842,924, filed Jul. 3, 2013, which is incorporated herein by reference.

### BACKGROUND OF THE INVENTION

This invention relates to detection or monitoring or both of Shiga toxin producing E. coli (“STEC”).

There are more than 200 Shiga toxin (stx)-producing Escherichia coli (“STEC”) serotypes, but many have not been implicated in causing illness. STEC may cause devastating illnesses, particularly in children, of varying severity, from diarrhea (often bloody), hemorrhagic colitis, and abdominal cramps to kidney disorders. Outbreaks of illnesses caused by STEC have been epidemiologically related to contact with animals and consumption of meat and fresh produce. Shiga toxin will bind to tissues in the kidneys and cause hemolytic uremic syndrome (“HUS”), leading to kidney failure and death. STEC also may cause asymptomatic infections and extraintestinal infections. Enterohemorrhagic E. coli (“EHEC”) is a subset of STEC and includes well recognized human pathogens. EHEC infections, like STEC infections, result in hemorrhagic colitis, which may progress into life-threatening HUS. E. coli O157:H7 is the most notorious STEC/EHEC strain most often associated with the most severe forms of disease. O157:H7 is a known food-borne pathogen increasingly causing illness worldwide.

Numerous non-O157 STEC isolates have also been linked to illnesses and outbreaks of disease. Six O groups have been described by the U.S. Center for Disease Control (“CDC”) to be the cause of the majority of non-O157 STEC disease. These serotypes have been identified as O26, O45, O103, O111, O121, and O145, and are commonly referred to as the “big six” non-O157 STEC. It is estimated that non-O157 STEC may cause diarrhea at frequencies similar to other enteric bacterial pathogens, such as Salmonella and Shigella. Non-O157 STEC also causes infections resulting in HUS.

The morbidity and mortality associated with worldwide outbreaks of STEC disease have highlighted the threat these organisms pose to public health. For this reason, there is a demand for compositions and diagnostic methods for detection of STEC in environmental and biological samples and, in particular, in foods such as meat and dairy products. Accordingly, there remains a need in the art for a rapid and robust detection system that can specifically and selectively identify virulent E. coli STEC in a sample of interest including virulent non-O157:H7 STECs O26, O45, O103, O111, O121, and O145.

Furthermore, E. coli O157:H7 and non-O157 shiga toxin-producing E. coli (STEC) strains are associated with severe illnesses such as hemorrhagic colitis (HC) and as mentioned above HUS, and have become an increasing concern to the beef industry, regulatory agencies, and the public (Bosilevac et al. 2011. Appl Environ Microbiol 77:2103-2112.). The U.S. Department of Agriculture Food Safety and Inspection Service (USDA FSIS) has determined, in addition to E. coli O157:H7, six most frequent STEC serogroups are adulterants in raw, non-intact beef products or components of such products. These six most frequent O serogroups were identified by the U.S. Centers for Disease Control and Prevention (CDC) as the most common non-O157 STEC responsible for 70% of all reported illness (Brooks et al. 2005. The Journal of infectious diseases 192:1422-1429.). The remaining 30% of illnesses, however, were caused by STEC of different O serogroups and are overlooked by current regulations. FSIS-based methods and many commercially available test methods screen initially for the presence of shiga toxin genes, stx1 and stx2, in addition to the locus of enterocyte effacement (LEE)-encoded intimin gene (eae). Presumptive positive samples are further analyzed for the presence of the six most frequent O serogroups O26, O45, O103, O111, O121, and O145 (USDA. 2013. Microbiology Laboratory Guidebook). These methods have the risk of detecting false positive results due to samples co-contaminated with two independent micro-organisms, each containing only one of the two target genes, stx and eae. Therefore, identification of single genetic markers that detect pathogenic STEC are likely to improve testing results by reducing undesirable false positive results.

### SUMMARY OF THE INVENTION

In general, as is described herein, the invention features a method for testing a sample for the presence of a virulent Escherichia coli, the method including detecting the presence of (i) ecf (e.g., the ecf operon, ecf1, ecf2, ecf3, and ecf4) and (ii) wzx and/or stx (e.g., stx1 or stx2 or any stx described herein) in the sample, wherein detection of (i) ecf and (ii) wzx and/or stx in the sample is taken as an indication that the sample includes the virulent E. coli strain. In these aspects, the detection of (i) and/or (ii) can be detection of a nucleic acid encoding (i) ecf and/or (ii) wzx and/or stx (e.g., stx1 or stx2 or any stx described herein). In another embodiment, detection of (i) or (ii) can include detection of an (i) ecf (e.g., ecf1, ecf2, ecf3, and ecf4) polypeptide and/or (ii) a wzx polypeptide and/or stx (e.g., stx1 or stx2) polypeptide.

Exemplary samples include virtually any material possibly contaminated with an E. coli pathogen. Samples include any food, water, biological, environmental or pharmaceutical sample as disclosed herein. Virtually any sample suspected of being contaminated with a virulent E. coli is tested using the methods and compositions described in this application. Exemplary samples include pharmaceutical, environmental (e.g., air, soil, lakes, rivers, or other water samples including sewage) or agricultural samples (e.g., those collected from agricultural watersheds as well as those collected from field and farm environments), samples obtained from cattle or other livestock including chickens and turkeys (such as during live animal production or during animal harvest), finished food products (e.g., for human or animal consumption), food ingredients and raw food materials, food samples (e.g., drinks and beverages (unpasteurized fresh-pressed juices such as apple cider), dairy products, yogurt, and cheese made from raw milk as well as raw, frozen, or processed foods), meat samples (e.g., raw ground beef, high fat ground beef, raw ground beef components (e.g., beef and veal bulk packed manufacturing trimmings and other beef and veal components such as primal cuts, sub primal cuts, head meat, cheek meat, esophagus meat, heart, and advanced meat recovery product intended for grinding)), produce such as fruits (e.g., grapes, apples, peaches, or strawberries), vegetables (e.g., lettuce, spinach, cabbage, celery, cilantro, coriander, cress sprouts, radishes, or alfalfa sprouts), as well as biological samples (e.g., fecal and blood samples) or samples from a food processing environment. Samples may also be collected to investigate foodborne outbreaks such as those originating in a restaurant or a food processing plant. Other samples are collected to facilitate checking the safety of a foodstuff suspected of being contaminated by a pathogen. Such a foodstuff may be for human or animal consumption, and may be in the form of a food or a beverage. Samples may be enriched as desired according to standard methods. The methods provide for testing to determine the presence or absence of the markers described herein according to standard techniques well known in the art.

In some embodiments of this invention, the detection of ecf and wzx is taken as an indication of the presence of E. coli O157:H7. In these or other embodiments, detection of ecf and the absence of wzx is taken as an indication of the presence of non-O157:H7 shiga toxin (stx)-containing E. coli (STEC). In these or yet other embodiments, detection of ecf and stx is taken as an indication of the presence of enterohemorrhagic Escherichia coli (EHEC).

In certain embodiments, the sample is obtained following enrichment of the sample, such as high fat ground beef, beef trim, or produce (for example, fruits such as grapes, apples, peaches, or strawberries and/or vegetables such as lettuce, spinach, radishes and alfalfa sprouts).

In embodiments which include detection of a nucleic acid, the detecting can include, e.g., contacting the sample with an oligonucleotide (e.g., an oligonucleotide with a detectable label) that hybridizes to a portion of a nucleic acid encoding the ecf operon, a nucleic acid encoding wzx, a nucleic acid encoding stx1, or a nucleic acid encoding stx2. These detection methods may include a hybridization assay selected from the group including of a Transcription Mediated Amplification (TMA) reaction, a Nucleic Acid Sequence-Based Amplification (NASBA) reaction, a Polymerase Chain Reaction (PCR) reaction, a hybridization protection assay, or a non-amplified hybridization reaction.

In embodiments which include detection of a polypeptide, the method may include a polypeptide detection assay, e.g., an immunoassay. The polypeptide detection methods generally include, e.g., contacting the sample with a molecule (e.g., a molecule with a detectable label) that specifically binds to a polypeptide selected from the group including of ecf1, ecf2, ecf3, ecf4, wzx, stx1, and stx2. Examples of such molecules include an antibody or fragment thereof.

In another aspect, the invention features a method for producing a packaged foodstuff free of a virulent Escherichia coli adulterant, the method including the steps of a) providing a sample obtained from a foodstuff; b) testing the foodstuff for the presence of (i) ecf (e.g., the ecf operon, ecf1, ecf2, ecf3, and ecf4) and (ii) wzx and/or stx (e.g., stx1 or stx2) in the sample, wherein absence of (i) ecf and (ii) wzx and/or stx in the sample is taken as an indication that the sample is free of pathogenic E. coli adulterant; and c) packaging the foodstuff identified as free of the pathogenic E. coli adulterant (e.g., packaging the foodstuff in a carton, container, plastic wrap, or a foodstuff tray wrapped with plastic).

In another aspect, the invention features a method for producing a packaged lot of meat free of a virulent Escherichia coli adulterant, the method including the steps of a) providing a sample obtained from a lot of meat (e.g., where the sample is obtained following enrichment of a meat sample);

b) testing the sample for the presence of (i) ecf (e.g., the ecf operon, ecf1, ecf2, ecf3, and ecf4) and (ii) wzx and/or stx (e.g., stx1 or stx2) in the sample, wherein absence of (i) ecf and (ii) wzx and/or stx in the sample is taken as an indication that the sample is free of pathogenic E. coli adulterant; and
c) packaging meat identified as free of the pathogenic E. coli adulterant (e.g., packaging the meat in a carton, container, plastic wrap, or a meat tray wrapped with plastic).

In these aspects, the detection of (i) and/or (ii) can be detection of a nucleic acid encoding (i) ecf (e.g., the ecf operon, ecf1, ecf2, ecf3, and ecf4) and/or (ii) wzx and/or stx (e.g., stx1 or stx2). In another embodiment, detection of (i) or (ii) can include detection of (i) an ecf (e.g., ecf1, ecf2, ecf3, and ecf4) polypeptide and/or (ii) a wzx polypeptide and/or stx (e.g., stx1 or stx2) polypeptide.

In some embodiments of this invention, the detection of ecf and wzx is taken as an indication of the presence of E. coli O157:H7. In these or other embodiments, detection ecf and the absence of wzx is taken as an indication of the presence of non-O157:H7 shiga toxin (stx)-containing E. coli (STEC). In these or yet other embodiments detection of ecf and stx is taken as an indication of the presence of enterohemorrhagic Escherichia coli (EHEC).

In embodiments which include detection of a nucleic acid, the detecting can include, e.g., contacting the sample with an oligonucleotide (e.g., an oligonucleotide with a detectable label) that hybridizes to a portion of a nucleic acid encoding the ecf operon, a nucleic acid encoding wzx, a nucleic acid encoding stx1, or a nucleic acid encoding stx2. These detection methods may include a hybridization assay selected from the group including of a Transcription Mediated Amplification (TMA) reaction, a Nucleic Acid Sequence-Based Amplification (NASBA) reaction, a Polymerase Chain Reaction (PCR) reaction, a hybridization protection assay, or a non-amplified hybridization reaction.

In embodiments which include detection of a polypeptide, the method may include a polypeptide detection assay, e.g., an immunoassay. The polypeptide detection methods can also include, e.g., contacting the sample with a molecule (e.g., a molecule with a detectable label) that specifically binds to a polypeptide selected from the group including of ecf1, ecf2, ecf3, ecf4, wzx, stx1, and stx2 or any stx described herein. Examples of such molecules include an antibody or fragment thereof.

In certain embodiments, the foregoing methods can, e.g., further include shipping the packaged meat. Also, the lot of meat can include, e.g., raw ground beef, high fat ground beef, raw ground beef components (e.g., beef and veal bulk packed manufacturing trimmings and other beef and veal components such as primal cuts, sub primal cuts, head meat, cheek meat, esophagus meat, heart, and advanced meat recovery product intended for grinding).

In any of the foregoing aspects, sample provided for enrichment is, e.g., about 200 g to about 500 g (e.g., about 325 g to about 375 g).

In another aspect, the invention features a method for producing a lot of produce free of a pathogenic Escherichia coli adulterant, the method including the steps of a) providing a sample obtained from a lot of produce (e.g., where the sample is obtained following enrichment of a produce sample);

b) testing for the presence of (i) ecf (e.g., the ecf operon, ecf1, ecf2, ecf3, and ecf4) and (ii) wzx and/or stx (e.g., stx1 or stx2 or any stx described herein) in the sample, wherein absence of (i) ecf and (ii) wzx and/or stx in the sample is taken as an indication that the sample is free of pathogenic E. coli adulterant; and c) packaging produce identified as free of the pathogenic E. coli adulterant.

In these aspects, the detection of (i) and/or (ii) can be detection of a nucleic acid encoding (i) ecf (e.g., the ecf operon, ecf1, ecf2, ecf3, and ecf4) and/or (ii) wzx and/or stx (e.g., stx1 or stx2). In another embodiment, detection of (i) or (ii) can include detection of (i) an ecf (e.g., ecf1, ecf2, ecf3, and ecf4) polypeptide and/or (ii) a wzx polypeptide and/or stx (e.g., stx1 or stx2) polypeptide.

In some embodiments of this invention, the detection of ecf and wzx is taken as an indication of the presence of E. coli O157:H7. In these or other embodiments, detection ecf and the absence of wzx is taken as an indication of the presence of non-O157:H7 shiga toxin (stx)-containing E. coli (STEC). In these or yet other embodiments detection of ecf and stx is taken as an indication of the presence of enterohemorrhagic Escherichia coli (EHEC).

In embodiments which include detection of a nucleic acid, the detecting can include, e.g., contacting the sample with an oligonucleotide (e.g., an oligonucleotide with a detectable label) that hybridizes to a portion of a nucleic acid encoding the ecf operon, a nucleic acid encoding wzx, a nucleic acid encoding stx1, or a nucleic acid encoding stx2. These detection methods may include a hybridization assay selected from the group including of a Transcription Mediated Amplification (TMA) reaction, a Nucleic Acid Sequence-Based Amplification (NASBA) reaction, a Polymerase Chain Reaction (PCR) reaction, a hybridization protection assay, or a non-amplified hybridization reaction.

In embodiments which include detection of a polypeptide, the method may include a polypeptide detection assay, e.g., an immunoassay. The polypeptide detection methods can also include, e.g., contacting the sample with a molecule (e.g., a molecule with a detectable label) that specifically binds to a polypeptide selected from the group including of ecf1, ecf2, ecf3, ecf4, wzx, stx1, and stx2. Examples of such molecules include an antibody or fragment thereof.

In certain embodiments, the foregoing methods can, e.g., further include shipping the packaged produce. Also, the lot of produce can include, e.g., fruit or vegetables (such as lettuce, spinach, cabbage, celery, cilantro, coriander, cress sprouts, radishes, or alfalfa sprouts).

In any of the foregoing aspects, sample provided for enrichment is, e.g., about 200 g to about 500 g (e.g., about 325 g to about 375 g).

In any of the foregoing methods, the detecting of (i) and detecting of (ii) can be performed in a single or multiple reaction mixtures.

In another aspect, the invention features a composition including (i) a first oligonucleotide that specifically hybridizes to a nucleic acid encoding the ecf operon, or portion thereof (e.g., ecf1, ecf2, ecf3, or ecf4), and (ii) a second oligonucleotide that specifically hybridizes to a nucleic acid encoding wzx, stx1, or stx2. In certain embodiments, the first and or second oligonucleotide can be, e.g., detectably labeled. The foregoing compositions can, e.g., further including primers for performing a Transcription Mediated Amplification (TMA) reaction, a Nucleic Acid Sequence-Based Amplification (NASBA) reaction and/or a Polymerase Chain Reaction (PCR) reaction.

In yet another aspect, the invention features a composition including (i) a first amplicon produced by a method of amplifying a nucleic acid encoding the ecf operon (e.g., ecf1, ecf2, ecf3, or ecf4) and (ii) a second amplicon produced by a method of amplifying a nucleic acid encoding wzx, stx1, or stx2. In certain embodiments, the method of amplifying the nucleic acid is selected from Transcription Mediated Amplification (TMA) reaction, a Nucleic Acid Sequence-Based Amplification (NASBA) reaction and a Polymerase Chain Reaction (PCR) reaction. In the foregoing compositions, the first and/or second amplicon can be, e.g., detectably labeled.

The invention also relates to the use of ECF such as the ecf operon/gene cluster (e.g., ECF2-1 and ECF2-2 described herein) to detect virulent STECs including virulent non-O157:H7 STEC and virulent non-O157:H7 EHEC. Use of this nucleic acid target, in combination, with other targets such as Z5866, rfb_{O157}, wzx_{O157}, wzy_{O157}, Z0344, Z0372, SIL_{O157}, and katP junction provides a robust, sensitive assay for distinguishing O157:H7 from virulent non-O157:H7 STEC.

The invention accordingly relates to compositions, kits, and methods used for the detection of E. coli STEC. The invention is based at least in part on the discovery that certain E. coli sequences are surprisingly efficacious for the detection of O157:H7 and virulent non-O157 STECs such as the big six: O26, O45, O103, O111, O121, and O145. In certain aspects and embodiments, particular regions of O157:H7 STEC have been identified as useful targets for nucleic acid amplification and, which when used in combination, provide improvements in relation to specificity, sensitivity, or speed of detection as well as other advantages.

By “virulent non-O157:H7 STEC” is meant any E. coli bacterium containing an Ecf gene cluster other than O157:H7. Exemplary virulent non-O157:H7 STEC include E. coli such as O26, O45, O103, O111, O121, and O145. Other exemplary non-O157:H7 STEC are those containing stx1 or stx2 in combination with eae and the large EHEC plasmid.

The invention accordingly further features a first method for assigning whether a sample includes Shiga-toxin producing E. coli (STEC), the method includes the steps of: (a) providing nucleic acids from a sample; (b) detecting an O157-specific fragment and an ECF-specific fragment; (c) assigning to the sample one of the following outcomes: 1) if the O157-specific fragment and the ECF-specific fragment are absent then the sample is negative for virulent O157 STEC and a virulent non-O157:H7 STEC; 2) if the O157-specific fragment is present and the ECF-specific fragment is absent then the sample is negative for a virulent non-O157:H7 STEC; 3) if the O157-specific fragment and ECF-specific fragment are present then the sample includes virulent O157 STEC; or 4) if the O157-specific fragment is absent and the ECF-specific fragment is present then the sample includes a virulent non-O157:H7 STEC. This method typically includes an O157-specific fragment which is rfb, wzx, or wzy as is disclosed herein. Exemplary virulent O157 STEC include O157:H7, O157:NM, O157:H−, O157:H8, or O157:H21. And exemplary virulent, non-O157:H7 STEC includes O26, O45, O103, O111, O121, or O145. The method also involves detection of at least two O157-specific fragments (e.g., rfb and wzk, rfb and wzy, and wzk and wzy, or rfb, wzk, and wzy). Other exemplary O157-specific fragments include katP junction and Z5866.

In another aspect, the invention features a second method for assigning whether a sample includes STEC, the method includes the steps of: (a) providing nucleic acids from a sample; (b) detecting an O157:H7-specific fragment and a ECF-specific fragment; (c) assigning to the sample one of the following outcomes: 1) if the O157:H7-specific fragment and the ECF-specific fragment are absent then the sample is negative for O157:H7 STEC and a virulent non-O157:H7 STEC is present; 2) if the O157:H7-specific fragment is present and the ECF-specific fragment is absent then the sample is negative for a virulent non-O157:H7 STEC; 3) if the O157:H7-specific fragment and the ECF-specific fragment are both present then the sample includes an O157:H7 STEC; or 4) if the O157:H7-specific fragment is absent and the ECF-specific fragment is present then the sample includes a virulent non-O157:H7 STEC. Exemplary O157:H7-specific fragments include katP junction or Z5866 as is described herein. Exemplary virulent, non-O157:H7 STEC includes O26, O45, O103, O111, O121, or O145. The method also involves, in certain embodiments, detection of at least two O157:H7-specific fragments.

In another aspect, the invention features a third method of assigning whether a sample includes STEC, the method includes the steps of: (a) providing nucleic acids from a sample; (b) detecting a first fragment that detects O157 STEC and STEC lacking an ECF gene, and a second fragment that detects an ECF gene; (c) assigning to the sample one of the following outcomes: 1) if the first and second fragments are absent then the sample is negative for virulent O157 STEC and a virulent non-O157:H7 STEC; 2) if the first fragment is present and the second fragment is absent then the sample is negative for a virulent non-O157:H7 STEC; 3) if the first fragment and second fragment are present then the sample includes virulent O157 STEC; or 4) if the first fragment is absent and the second fragment is present then the sample includes a virulent non-O157:H7 STEC. Exemplary first fragments include Sil or Z0372, as is described herein. Exemplary virulent O157 STEC includes O157:H7, O157:NM, O157:H−, O157:H8, or O157:H21. And exemplary virulent, non-O157:H7 STEC includes O26, O45, O103, O111, O121, or O145. The method also involves detection of at least two first fragments (e.g., Sil and Z0372).

In another aspect, the invention features a fourth method of assigning whether a sample includes STEC, the method includes the steps of: (a) providing nucleic acids from a sample; (b) detecting a first fragment that detects O157:H7 STEC and STEC lacking an ECF gene, and a second fragment that detects the ECF gene; (c) assigning to the sample one of the following outcomes: 1) if the first and second fragments are absent then the sample is negative for O157:H7 STEC and a virulent non-O157:H7 STEC; 2) if the first fragment is present and the second fragment is absent then the sample is negative for virulent non-O157:H7 STEC; 3) if the first fragment and second fragment are present then the sample includes an O157:H7 STEC; or 4) if the first fragment is absent and the second fragment is present then the sample includes a virulent non-O157:H7 STEC. Exemplary virulent, non-O157:H7 STEC includes O26, O45, O103, O111, O121, or O145.

In another aspect, the invention features still a method for detecting STEC in a sample, the method including the steps of: a) providing a sample including nucleic acid molecules; b) contacting the nucleic acid molecules with a virulent O157 STEC-specific probe and an ECF-specific probe under hybridization conditions, wherein i) the virulent O157 STEC-specific probe specifically hybridizes to a virulent O157 STEC-specific fragment of the nucleic acid molecules; and ii) the ECF-specific probe specifically hybridizes to an ECF-specific fragment of the nucleic acid molecules; and c) detecting hybridization of the virulent O157 STEC-specific probe and the ECF-specific probe to identify the presence or absence of the virulent O157 STEC-specific fragment or the ECF-specific fragment as an indication of the presence of absence of STEC in the sample. Typically, the absence of the virulent O157 STEC-specific fragment and absence of the ECF-specific fragment is taken as an indication that the sample is negative for virulent O157 STEC and a virulent non-O157:H7 STEC; the presence of the virulent O157-specific fragment and the absence of the ECF-specific fragment is taken as an indication that the sample is negative for a virulent non-O157:H7 STEC; the presence of the virulent O157-specific fragment and the presence of the ECF-specific fragment is taken as an indication that the sample is positive for virulent O157 STEC; or the absence of the virulent O157 STEC-specific fragment and the presence of the ECF-specific fragment is taken as an indication that the sample is positive for a virulent non-O157:H7 STEC. Exemplary virulent O157 STEC-specific fragments include rfb, wzx, or wzy. Exemplary virulent O157 STEC includes O157:H7, O157:NM, O157:H−, O157:H8, or O157:H21. And exemplary virulent, non-O157:H7 STEC includes O26, O45, O103, O111, O121, or O145. The method also involves detection of at least two virulent O157 STEC-specific fragments (e.g., rfb and wzk, rfb and wzy, and wzk and wzy, or rfb, wzk, and wzy). Exemplary methods for detecting hybridization involve amplification or cDNA synthesis. Nucleic acid molecules, if desired, are typically purified from an environmental or a biological sample (e.g., a food sample such as meat).

In another aspect, the invention features a method for detecting STEC in a sample, the method includes the steps of: a) providing a sample including nucleic acid molecules; b) contacting the nucleic acid molecules with an O157:H7-specific probe and an ECF-specific probe under hybridization conditions, wherein i) the O157:H7-specific probe specifically hybridizes to an O157:H7-specific fragment of the nucleic acid molecules; and ii) the ECF-specific probe specifically hybridizes to an ECF-specific fragment of the nucleic acid molecules; and c) detecting hybridization of the O157:H7-specific probe and the ECF-specific probe to identify the presence or absence of the O157:H7-specific fragment or the ECF-specific fragment as an indication of the presence of absence of STEC in the sample. Typically, the absence of the O157:H7-specific fragment and absence of the ECF-specific fragment is taken as an indication that the sample is negative for O157:H7 STEC and a virulent non-O157:H7 STEC; the presence of the O157:H7-specific fragment and the absence of the ECF-specific fragment is taken as an indication that the sample is negative for a virulent non-O157:H7 STEC; the presence of the O157:H7-specific fragment and the presence of the ECF-specific fragment is taken as an indication that the sample is positive for an O157:H7 STEC; or the absence of the O157:H7-specific fragment and the absence of the ECF-specific fragment is taken as an indication that the sample is positive for a virulent non-O157:H7 STEC. Exemplary O157:H7-specific fragments include katP junction or Z5866 as is described herein.

Exemplary virulent, non-O157:H7 STEC include O26, O45, O103, O111, O121, or O145. The method also involves detection of at least two O157:H7-specific fragments (e.g, katP and Z5866). Standard methods for detecting hybridization involve amplification or cDNA synthesis. Nucleic acid molecules, if desired, are typically purified from an environmental or a biological sample (e.g., a food sample such as meat).

In another aspect, the invention features a method for detecting STEC in a sample, the method includes the steps of: a) providing a sample including nucleic acid molecules; b) contacting the nucleic acid molecules with a first probe and a second probe under hybridization conditions, wherein i) the first probe specifically hybridizes with nucleic acid molecules of (1) a virulent O157 STEC and (2) STEC lacking an ECF gene; and ii) the second probe specifically hybridizes to an ECF-specific fragment of the nucleic acid molecules; and c) detecting hybridization of the first probe and the second probe, wherein the presence or absence of hybridization to the first probe and the second probe is taken as indication of the presence or absence of STEC in the sample. Typically, the absence of hybridization to the first probe and absence of hybridization to the second probe is taken as an indication that the sample is negative for virulent O157 STEC and a virulent non-O157:H7 STEC; the presence of hybridization to the first probe and the absence of hybridization to the second probe is taken as an indication that the sample is negative for a virulent non-O157:H7 STEC; the presence of hybridization to the first probe and the presence of hybridization to the second probe is taken as an indication that the sample is positive for virulent O157 STEC; or the absence of hybridization to the first probe and the presence of hybridization to the second probe is taken as an indication that the sample is positive for a virulent non-O157:H7 STEC. Exemplary first fragments include Sil or Z0372 as is described herein. Exemplary virulent O157 STEC includes O157:H7, O157:NM, O157:H−, O157:H8, or O157:H21. Exemplary virulent, non-O157:H7 STEC includes O26, O45, O103, O111, O121, or O145. The method also involves detection of at least two first fragments (e.g., Sil and Z0372). Standard methods for detecting hybridization involve amplification or cDNA synthesis. Nucleic acid molecules, if desired, are typically purified from an environmental or a biological sample (e.g. a food sample such as meat).

In still another aspect, the invention features an method for detecting STEC in a sample, the method including the steps of: a) providing a sample including nucleic acid molecules; b) contacting the nucleic acid molecules with a first probe and a second probe under hybridization conditions, wherein i) the first probe specifically hybridizes with nucleic acid molecules of (1) an O157:H7 STEC and (2) STEC lacking an ECF gene; and ii) the second probe specifically hybridizes to an ECF-specific fragment of the nucleic acid molecules; and c) detecting hybridization of the first probe and the second probe, wherein the presence or absence of hybridization to the first probe and the second probe is taken as indication of the presence or absence of STEC in the sample. Typically, the absence of hybridization to the first probe and absence of hybridization to the second probe is taken as an indication that the sample is negative for O157 STEC and a virulent non-O157:H7 STEC; the presence of hybridization to the first probe and the absence of hybridization to the second probe is taken as an indication that the sample is negative for a virulent non-O157:H7 STEC; the presence of hybridization to the first probe and the presence of hybridization to the second probe is taken as an indication that the sample is positive for an O157:H7 STEC; or the absence of hybridization to the first probe and the presence of hybridization to the second probe is taken as an indication that the sample is positive for a virulent non-O157:H7 STEC. Standard methods for detecting hybridization involve amplification or cDNA synthesis. Nucleic acid molecules, if desired, are typically purified from an environmental or a biological sample (e.g., a food sample such as meat).

In another aspect, the invention features a method for assessing the presence or absence of virulent non-O157:H7 STEC in a sample, the method includes the steps of: a) contacting nucleic acid molecules from the sample with an ECF-specific probe under hybridization conditions, wherein the ECF-specific probe specifically hybridizes to an ECF-specific region; and b) detecting hybridization of the ECF-specific probe and the nucleic acid molecules, wherein presence or absence of hybridization of the ECF-specific probe with the nucleic acid molecules indicates the presence or absence of virulent non-O157:H7 STEC in the sample. Typically, the nucleic acid molecules are contacted with a virulent O157 STEC-specific probe that specifically hybridizes to a virulent O157 STEC-specific fragment of the nucleic acid molecules, and wherein (i) absence of hybridization of the O157 STEC-specific probe and absence of hybridization of the ECF-specific probe is taken as an indication that the sample is negative for virulent O157 STEC and a virulent non-O157:H7 STEC; (ii) the presence of hybridization of the virulent O157-specific fragment and the absence of hybridization of the ECF-specific fragment is taken as an indication that the sample is negative for a virulent non-O157:H7 STEC; (iii) the presence of hybridization of the virulent O157-specific fragment and the presence of hybridization of the ECF-specific fragment is taken as an indication that the sample is positive for virulent O157 STEC; or (iv) the absence of hybridization of the virulent O157 STEC-specific fragment and the presence of hybridization of the ECF-specific fragment is taken as an indication that the sample is positive for a virulent non-O157:H7 STEC. The nucleic acid molecules may also be contacted with a O157:H7-specific probe that specifically hybridizes to an O157:H7-specific fragment of the nucleic acid molecules, and (i) the absence of hybridization of the O157:H7-specific fragment and absence of hybridization of the ECF-specific fragment is taken as an indication that the sample is negative for O157:H7 STEC and a virulent non-O157:H7 STEC; (ii) the presence of hybridization of the O157:H7-specific fragment and the absence of hybridization of the ECF-specific fragment is taken as an indication that the sample is negative for a virulent non-O157:H7 STEC; (iii) the presence of hybridization of the O157:H7-specific fragment and the presence of hybridization of the ECF-specific fragment is taken as an indication that the sample is positive for an O157:H7 STEC; and (iv) the absence of hybridization of the O157:H7-specific fragment and the absence of the ECF-specific fragment is taken as an indication that the sample is positive for a virulent non-O157:H7 STEC.

Similarly, the nucleic acid molecules may be contacted with a probe (a′) that specifically hybridizes with nucleic acid molecules of (1) a virulent O157 STEC and (2) STEC lacking an ECF gene; and wherein (i) the absence of hybridization to the probe (a′) and absence of hybridization to the ECF-specific fragment is taken as an indication that the sample is negative for virulent O157 STEC and a virulent non-O157:H7 STEC, (ii) the presence of hybridization to the probe (a′) and the absence of hybridization to the ECF-specific fragment is taken as an indication that the sample is negative for a virulent non-O157:H7 STEC; (iii) the presence of hybridization to the probe (a′) and the presence of hybridization to the ECF-specific fragment is taken as an indication that the sample is positive for virulent O157 STEC, (iv) the absence of hybridization to the probe (a′) and the presence of hybridization to the ECF-specific fragment is taken as an indication that the sample is positive for a virulent non-O157:H7 STEC.

And, if desired, the nucleic acid molecules may be contacted with a probe (b′) that specifically hybridizes with nucleic acid molecules of (1) an O157:H7 STEC and (2) STEC lacking an ECF gene, and wherein (i) the absence of hybridization to probe (b′) and absence of hybridization to the ECF-specific fragment is taken as an indication that the sample is negative for O157 STEC and a virulent non-O157:H7 STEC; (ii) the presence of hybridization to the probe (b′) and the absence of hybridization to the ECF-specific fragment is taken as an indication that the sample is negative for a virulent non-O157:H7 STEC, (iii) the presence of hybridization to the probe (b′) and the presence of hybridization to the ECF-specific fragment is taken as an indication that the sample is positive for an O157:H7 STEC, and (iv) the absence of hybridization to the probe (b′) and the presence of hybridization to the ECF-specific fragment is taken as an indication that the sample is positive for a virulent non-O157:H7 STEC.

In still another number of aspects, the invention features targets for identifying a STEC as well as oligonucleotides or primers, alone or in combination, which are useful for identifying or amplifying such targets. Exemplary target sequences and oligonucleotides are described herein (see, for example, FIGS. 1-9 and Table 2 as well as other sequences described herein, respectively).

Accordingly, in another aspect, the invention features a nucleic acid consisting of a nucleic acid sequence wherein the nucleic acid sequence is a 1318 bp Z5886 shown in FIG. 1 or a fragment thereof or sequence complementary thereto.

In another aspect, the invention features a composition including a nucleic acid consisting of a nucleic acid sequence wherein the nucleic acid sequence is a fragment of the Ecf gene cluster shown in FIG. 2 or a fragment thereof or sequence complementary thereto, wherein the fragment is 1-2404 bp or 3584-5612 bp as shown in FIG. 2. Exemplary nucleic acid sequences are the 949 bp Ecf2-1 fragment or the 1050 bp Ecf2-2 fragment, each disclosed herein. For example, an isolated nucleic acid sequence selected from the group consisting of: 5′-CCC TTA TGA AGA GCC AGT ACT GAA G-3′ (SEQ ID NO: 1) and 5′ ATT ACG CAT AGG GCG TAT CAG CAC-3′ (SEQ ID NO: 2).

Other Ecf primers include the following or combinations thereof:

| | | SEQ |
|---|---|---|
| | | ID |
| | Sequence | NO: |
| ecf1 Set 1 | CCC TTA TGA AGA | 1 |
| Forward | GCC AGT ACT | |
| Primer | GAAG | |
| ecf1 Set 1 | ATT ACG CAT AGG | 2 |
| Reverse | GCG TAT CAG CAC | |
| Primer | | |
| ecf1 Set 3 | TGC AAG GCA TCT | 3 |
| Forward | TCC CGT ACT GAT | |
| Primer | | |
| ecf1 Set 3 | TCT GCG AGC CAC | 4 |
| Reverse | TTC ATC TGT TCA | |
| Primer | | |
| ecf1 Set 5 | AGC AGG AAT ATT | 5 |
| Forward | CTC ACC GCG ACT | |
| Primer | | |
| ecf1 Set 5 | ACA GAC AAC CTG | 6 |
| Reverse | TCC CAG CGT TTA | |
| Primer | | |
| ecf3 Set 1 | TTC CTT TGC CAT | 7 |
| Forward | GGC GGA GAA TTG | |
| Primer | | |
| ecf3 Set 1 | AGC GGC TCC TGT | 8 |
| Reverse | CTG ATT AAC GAT | |
| Primer | | |
| ecf3 Set 4 | TGA TCA TCG TGC | 9 |
| Forward | ATC TGC TGG GTA | |
| Primer | | |
| ecf3 Set 4 | ATG CCC TGT AAT | 10 |
| Reverse | GCC ATC AAA CCG | |
| Primer | | |
| ecf3 Set 5 | TGT ACA CTG TTC | 11 |
| Forward | CGT TCC TGC TGT | |
| Primer | | |
| ecf3 Set 5 | TCC CTG AAT TGC | 12 |
| Reverse | GGA TTC ACC AGA | |
| Primer | | |
| ecf4 Set 3 | ACG CTG GAA TGG | 13 |
| Forward | TCT GGA GAT TGT | |
| Primer | | |
| ecf4 Set 3 | ATC CAC CAC CGG | 14 |
| Reverse | ATT TCT CTG GTT | |
| Primer | | |
| ecf4 Set 4 | AAC TTT ACC GGT | 15 |
| Forward | TAT CGG ACG GCT | |
| Primer | | |
| ecf4 Set 4 | TGC TCA GGA TGT | 16 |
| Reverse | GGA CGA ACG AAA | |
| Primer | | |
| ecf4 Set 1 | TGG TAC CAC CTT | 17 |
| Forward | CTG CTG TAC TCT | |
| Primer | | |
| ecf4 Set 1 | TAC CTG TCC ACG | 18 |
| Reverse | TCA TCC AGT AAC | |
| Primer | | |

In still another aspect, the invention features a composition including a nucleic acid consisting of a nucleic acid sequence wherein the nucleic acid sequence is a 1269 bp Rfb_{O157} shown in FIG. 3 or a fragment thereof or sequence complementary thereto. In another aspect, the invention features a composition including a nucleic acid consisting of a nucleic acid sequence wherein the nucleic acid sequence is a 1392 bp Wzx_{O157} shown in FIG. 4 or a fragment thereof or sequence complementary thereto.

In another aspect, the invention features a composition including a nucleic acid consisting of a nucleic acid sequence wherein the nucleic acid sequence is a 1185 bp Wzy_{O157} shown in FIG. 5 or a fragment thereof or sequence complementary thereto.

In yet another aspect, the invention features a composition including a nucleic acid consisting of a nucleic acid sequence wherein the nucleic acid sequence is a 2634 bp SIL_{O157} shown in FIG. 6 or a fragment thereof or sequence complementary thereto.

In another aspect, the invention features a composition including a nucleic acid consisting of a nucleic acid sequence wherein the nucleic acid sequence is a 279 bp Z0344 shown in FIG. 7 or a fragment thereof or sequence complementary thereto.

And in another aspect, the invention features a composition including a nucleic acid consisting of a nucleic acid sequence wherein the nucleic acid sequence is a 357 bp Z0372 shown in FIG. 8 or a fragment thereof or sequence complementary thereto.

The invention also features oligonucleotides that bind to any of the aforementioned targets as well as combinations of any of these oligonucleotides.

Accordingly, the invention further features a composition, including: a first oligonucleotide that has a target-complementary base sequence to Ecf2-1 or Ecf2-2, optionally including a 5′ sequence that is not complementary to the specific target sequence.

In addition, the invention features a composition, including: a first oligonucleotide that has a target-complementary base sequence to Ecf gene cluster, optionally including a 5′ sequence that is not complementary to the specific target sequence and a second oligonucleotide. Exemplary second oligonuclotides include, without limitation, an oligonucleotide selected from the group consisting of:
a.) an oligonucleotide that has a target-complementary base sequence to Z5886, optionally including a 5′ sequence that is not complementary to the specific target sequence;
b.) an oligonucleotide that has a target-complementary base sequence to hyIA, optionally including a 5′ sequence that is not complementary to the specific target sequence;
c.) an oligonucleotide that has a target-complementary base sequence to rfb_{O157}, optionally including a 5′ sequence that is not complementary to the specific target sequence;
d.) an oligonucleotide that has a target-complementary base sequence to wzx_{O157}, optionally including a 5′ sequence that is not complementary to the specific target sequence;
e.) an oligonucleotide that has a target-complementary base sequence to wzy_{O157}, optionally including a 5′ sequence that is not complementary to the specific target sequence;
f.) an oligonucleotide that has a target-complementary base sequence to SIL_{O157}, optionally including a 5′ sequence that is not complementary to the specific target sequence.
g.) an oligonucleotide that has a target-complementary base sequence to Z0344, optionally including a 5′ sequence that is not complementary to the specific target sequence;
h.) an oligonucleotide that has a target-complementary base sequence to Z0372, optionally including a 5′ sequence that is not complementary to the specific target sequence;
i.) an oligonucleotide that has a target-complementary base sequence to katP junction, optionally including a 5′ sequence that is not complementary to the specific target sequence.

Such compositions are prepared, if desired, so that only one of the first and second oligonucleotides has a 3′ end that can be extended by a template-dependent DNA polymerase. Further, if desired, an oligonucleotide may include a detectably labeled hybridization probe.

The invention provides long awaited advantages over a wide variety of standard screening methods used for distinguishing and evaluating STEC. In particular, the invention disclosed herein reduces not only the number of false positives typically obtained when compared to current methods but also reduces the number of tests and steps performed on a sample. The invention accordingly obviates many issues encountered when analyzing a sample in which many microorganism co-infections result in a high false positive rate.

Accordingly, the methods of the invention provide a facile means to identify and distinguish STEC. In addition, the methods of the invention provide a route for analyzing virtually any number of samples for presence of STEC with high-volume throughput and high sensitivity. The methods are also relatively inexpensive to perform and enable the analysis of small quantities of samples found in either purified or crude extract form.

Further, the invention disclosed herein advantageously demonstrates specificity for distinguishing highly virulent non-O157:H7 STEC, including the big six non-O157:H7 STECs, from O157:H7.

Other features and advantages of the invention will be apparent from the following description of the preferred embodiments thereof, and from the claims.

### BRIEF DESCRIPTION OF THE DRAWINGS

FIG. 1 shows a 1318 bp sequence of Z5886. Forward and reverse primers used to generate an 80 bp amplicon are also shown.

FIG. 2 shows a 5612 bp sequence of the ECF gene cluster as well as Ecf2-1 and Ecf2-2 fragments respectively 949 bp and 1050 bp. Forward and reverse primers used to generate a 114 bp amplicon are also shown in connection with the ECF gene cluster and Ecf2-1 gene fragment.

FIG. 3 shows a 1269 bp sequence of Rfb_{O157}. Forward and reverse primers used to generate a 141 bp amplicon are also shown.

FIG. 4 shows a 1392 bp sequence of wzx_{O157}. Forward and reverse primers used to generate a 122 bp amplicon are also shown. Forward and reverse primers used to generate a 167 bp amplicon are shown as well.

FIG. 5 shows a 1185 bp sequence of wzy. Forward and reverse primers used to generate a 191 bp amplicon are also shown.

FIG. 6 shows a 2634 bp sequence of SIL_{O157}. Forward and reverse primers used to generate a 152 bp amplicon are shown.

FIG. 7 shows a 279 bp sequence of Z0344. Forward and reverse primers used to generate a 125 bp amplicon are shown.

FIG. 8 shows a 357 bp sequence of Z0372. Forward and reverse primers used to generate a 177 bp amplicon are shown.

FIG. 9 shows a 1489 bp sequence of katP junction. Forward and reverse primers used to generate a 101 bp amplicon are shown.

FIG. 10 shows polymerase chain reaction (PCR) screening results testing 214 E. coli strains for identifying virulent O157:H7 and non-O157 STEC.

FIG. 11 shows ecf-1, ecf-2, ecf-3, and ecf-4 nucleotide and polypeptide sequences.

FIG. 12 shows WZX O-antigen nucleotide and polypeptide sequences.

FIG. 13 shows Shiga Toxin nucleotide and polypeptide sequences.

### DETAILED DESCRIPTION OF THE INVENTION

In certain aspects and embodiments, the invention relates to compositions, methods and kits for the identification, detection, and/or quantitation of E. coli STEC, which may be present either alone or as a component, large or small, of a homogeneous or heterogeneous mixture of nucleic acids in a sample taken for testing, e.g., for diagnostic testing, for screening of blood products, for microbiological detection in bioprocesses, food such as meat or dairy products, water, animals such as reservoirs of O157:H7 and non-O157:H7 STEC such as ruminants and other animals, industrial or environmental samples, and for other purposes. Specific methods, compositions, and kits as disclosed herein provide improved sensitivity, specificity, or speed of detection in the amplification-based detection of E. coli STEC such as O157:H7 and non-O157:H7 STEC. Accordingly, in certain embodiments of the invention, assays disclosed herein identify ecf sequences common to E. coli O157:H7 and non-O157:H7 STEC, and differentiates E. coli STECs including virulent non-O157 STECs such as O26, O45, O103, O111, O121, and O145 from other non-virulent strains and, for example, from O157:H7. A preferred useful region for such differentiation is the ECF gene cluster, for example Ecf2-1 and Ecf2-2.

As a result of extensive analyses of amplification oligonucleotides specific for E. coli O157:H7, the particular region of E. coli O157:H7, corresponding to the region of E. coli Ecf2-1 sequence, has been identified as a target for amplification-based detection of E. coli O157:H7 and non-O157:H7 STEC. In addition, after extensive analysis a particular region of E. coli O157:H7 (Z5886)(hereinafter referred to as the “Z5886 region”) has been identified as still another useful target for amplification-based detection of E. coli O157:H7. Other useful regions include rfb_{O157}, wzx_{O157}, wzy_{O157}, Z0344, Z0372, SIL_{O157}, and katP junction as is disclosed herein. Accordingly, the invention relates to methods of detection of E. coli O157:H7 and non-O157:H7 STEC in a sample of interest, amplification oligonucleotides, compositions, reaction mixtures, and kits.

The assays described herein detect sequences specific for STEC from other non-virulent strains. The assays also provide for the detection of the big six virulent, non-O157:H7 STEC. It may utilize virtually any known nucleic amplification protocol such as real-time polymerase chain reaction (PCR) or real-time transcription mediated amplification (TMA), where the target-specific sequence is amplified and a fluorescent molecular torch is used to detect the amplified products as they are produced. Target detection is performed simultaneously with the amplification and detection of an internal control in order to confirm reliability of the result. The result of the assay consists of the classification of the sample as positive or negative for the presence or absence of STEC.

In one embodiment, the sample is a blood sample or a contaminated meat product where STEC is a known or suspected contaminant. Using the methods disclosed herein, for example, the presence of STEC in one or more contaminated samples may be monitored in a rapid and sensitive fashion.

### Target Nucleic Acid/Target Sequence

Target nucleic acids may be isolated from any number of sources based on the purpose of the amplification assay being carried out. The present invention provides a method for detecting and distinguishing between E. coli (e.g., O157 STEC and virulent non-O157 strains) using a hybridization assay that may also include a nucleic amplification step that precedes a hybridization step. Preparation of samples for amplification of E. coli sequences may include separating and/or concentrating organisms contained in a sample from other sample components according to standard techniques, e.g., filtration of particulate matter from air, water, or other types of samples. Once separated or concentrated, the target nucleic acid may be obtained from any medium of interest, such as those described above and, in particular, contaminated food. Sample preparation may also include chemical, mechanical, and/or enzymatic disruption of cells to release intracellular contents, including E. coli RNA or DNA. Preferred samples are food and environmental samples. Methods to prepare target nucleic acids from various sources for amplification are well known to those of ordinary skill in the art. Target nucleic acids may be purified to some degree prior to the amplification reactions described herein, but in other cases, the sample is added to the amplification reaction without any further manipulations.

Sample preparation may include a step of target capture to specifically or non-specifically separate the target nucleic acids from other sample components. Nonspecific target preparation methods may selectively precipitate nucleic acids from a substantially aqueous mixture, adhere nucleic acids to a support that is washed to remove other sample components, or use other means to physically separate nucleic acids, including STEC nucleic acid, from a mixture that contains other components. Other nonspecific target preparation methods may selectively separate RNA from DNA in a sample.

A target sequence may be of any practical length. An optimal length of a target sequence depends on a number of considerations, for example, the amount of secondary structure, or self-hybridizing regions in the sequence. Typically, target sequences range from about 30 nucleotides in length to about 300 nucleotides in length or greater. Target sequences accordingly may range from 3-100, 50-150, 75-200, 100-500, or even 500-800 or 900-1,100 nucleotides in length. The optimal or preferred length may vary under different conditions which can be determined according to the methods described herein and the sequences of the targets described herein.

### Nucleic Acid Identity

In some instances, a nucleic acid comprises a contiguous base region that is at least 70%; or 75%; or 80%, or 85% or 90%, or 95%, or even 96%, 97%, 98%, 99% or even 100% identical to a contiguous base region of a reference nucleic acid. For short nucleic acids, the degree of identity between a base region of a query nucleic acid and a base region of a reference nucleic acid can be determined by manual alignment or using any standard alignment tool known in the art such as “BLAST.” “Identity’ is simply determined by comparing just the nucleic acid sequences. Thus, the query:reference base sequence alignment may be DNA:DNA, RNA:RNA, DNA:RNA, RNA:DNA, or any combinations or analogs thereof. Equivalent RNA and DNA base sequences can be compared by converting U's (in RNA) to T's (in DNA).

### Oligonucleotides

An oligonucleotide can be virtually any length, limited only by its specific function in the amplification reaction or in detecting an amplification product of the amplification reaction. However, in certain embodiments, preferred oligonucleotides will contain at least about 5, 6, 7, 8, 9, or 10; or 11, 12, 13, 14, 15, 16, 17, 18, 19 or 20; or 22; or 24; or 26; or 28; or 30; or 32; or 34; or 36; or 38; or 40; or 42; or 44; or 46; or 48; or 50; or 52; or 54; or 56 contiguous bases that are complementary to a region of the target nucleic acid sequence or its complementary strand. The contiguous bases are preferably at least about 80%, more preferably at least about 90%, and most preferably completely complementary to the target sequence to which the oligonucleotide binds. Certain preferred oligonucleotides are of lengths generally between about 5-20, 5-25, 10-100; or 12-75; or 14-50; or 15-40 bases long and optionally can include modified nucleotides. Exemplary oligonucleotides are described herein.

Oligonucleotides may be modified in any way, as long as a given modification is compatible with the desired function of a given oligonucleotide. One of ordinary skill in the art can easily determine whether a given modification is suitable or desired for any given oligonucleotide. Modifications include base modifications, sugar modifications or backbone modifications.

Primers are a type of oligonucleotide used in amplification reactions. Primers have a 3′ hydroxyl group which is involved in the amplification reaction.

### Nucleic Acid Amplification

Many well-known methods of nucleic acid amplification require thermocycling to alternately denature double-stranded nucleic acids and hybridize primers; however, other well-known methods of nucleic acid amplification are isothermal. Exemplary amplification methods include polymerase chain reaction (“PCR”), the ligase chain reaction (“LCR”), strand displacement amplification (“SDA”), nucleic acid sequence based amplification (“NASBA”), self-sustained sequence replication, and transcription-mediated amplification (“TMA”).

Suitable amplification conditions can be readily determined by a skilled artisan in view of the present disclosure. Amplification conditions, as disclosed herein, refer to conditions which permit nucleic acid amplification. Amplification conditions may, in some embodiments, be less stringent than “stringent hybridization conditions” as described herein. By “stringent hybridization conditions” is meant hybridization assay conditions wherein a specific detection probe is able to hybridize with target nucleic acids over other nucleic acids present in the test sample. It will be appreciated that these conditions may vary depending upon factors including the GC content and length of the probe, the hybridization temperature, the composition of the hybridization reagent or solution, and the degree of hybridization specificity sought. Specific stringent hybridization conditions are disclosed herein.

Oligonucleotides used in the amplification reactions as disclosed herein may be specific for and hybridize to their intended targets under amplification conditions, but in certain embodiments may or may not hybridize under more stringent hybridization conditions. On the other hand, detection probes generally hybridize under stringent hybridization conditions. While the Examples section infra provides preferred amplification conditions for amplifying target nucleic acid sequences, other acceptable conditions to carry out nucleic acid amplifications could be easily ascertained by someone having ordinary skill in the art depending on the particular method of amplification employed.

In a preferred embodiment, the target nucleic acid of a STEC can also be amplified by a transcription-based amplification technique. As is discussed above, one transcription-based amplification system is transcription-mediated amplification (TMA), which employs an RNA polymerase to produce multiple RNA transcripts of a target region. Exemplary TMA amplification methods are described in, e.g., U.S. Pat. Nos. 4,868,105; 5,124,246; 5,130,238; 5,399,491; 5,437,990; 5,480,784; 5,554,516; and 7,374,885; and PCT Pub. Nos. WO 88/01302; WO 88/10315 and WO 95/03430.

The methods of the present invention may include a TMA reaction that involves the use of a single primer TMA reaction, as is described in U.S. Pat. No. 7,374,885. In general, the single-primer TMA methods use a primer oligomer (e.g., a NT7 primer), a modified promoter-based oligomer (or “promoter-provider oligomer”; e.g., a T7 provider) that is modified to prevent the initiation of DNA synthesis from its 3′ end (e.g., by including a 3′-blocking moiety) and, optionally, a blocker oligomer (e.g., a blocker) to terminate elongation of a cDNA from the target strand. Promoter-based oligomers provide an oligonucleotide sequence that is recognized by an RNA polymerase. This single primer TMA method synthesizes multiple copies of a target sequence and includes the steps of treating a target RNA that contains a target sequence with a priming oligomer and a binding molecule, where the primer hybridizes to the 3′ end of the target strand. RT initiates primer extension from the 3′ end of the primer to produce a cDNA which is in a duplex with the target strand (e.g., RNA:cDNA). When a blocker oligomer, is used in the reaction, it binds to the target nucleic acid adjacent near the user designated 5′ end of the target sequence. When the primer is extended by DNA polymerase activity of RT to produce cDNA, the 3′ end of the cDNA is determined by the position of the blocker oligomer because polymerization stops when the primer extension product reaches the binding molecule bound to the target strand. Thus, the 3′ end of the cDNA is complementary to the 5′ end of the target sequence. The RNA:cDNA duplex is separated when RNase (e.g., RNase H of RT) degrades the RNA strand, although those skilled in the art will appreciate that any form of strand separation may be used. Then, the promoter-provider oligomer hybridizes to the cDNA near the 3′ end of the cDNA strand.

The promoter-provider oligomer includes a 5′ promoter sequence for an RNA polymerase and a 3′ target hybridizing region complementary to a sequence in the 3′ region of the cDNA. The promoter-provider oligomer also has a modified 3′ end that includes a blocking moiety that prevents initiation of DNA synthesis from the 3′ end of the promoter-provider oligomer. In the promoter-provider:cDNA duplex, the 3′-end of the cDNA is extended by DNA polymerase activity of RT using the promoter oligomer as a template to add a promoter sequence to the cDNA and create a functional double-stranded promoter.

An RNA polymerase specific for the promoter sequence then binds to the functional promoter and transcribes multiple RNA transcripts complementary to the cDNA and substantially identical to the target region sequence that was amplified from the initial target strand. The resulting amplified RNA can then cycle through the process again by binding the primer and serving as a template for further cDNA production, ultimately producing many amplicons from the initial target nucleic acid present in the sample. Some embodiments of the single-primer transcription-associated amplification method do not include the blocking oligomer and, therefore, the cDNA product made from the primer has an indeterminate 3′ end, but the amplification steps proceed substantially as described above for all other steps.

The methods of the invention may also utilize a reverse transcription-mediated amplification (RTMA), various aspects of which are disclosed in, e.g., U.S. Pat. Appln. Pub. No. US 2006-0046265 A1. RTMA is an RNA transcription-mediated amplification system using two enzymes to drive the reaction: RNA polymerase and reverse transcriptase. RTMA is isothermal; the entire reaction is performed at the same temperature in a water bath or heat block. This is in contrast to other amplification reactions such as PCR that require a thermal cycler instrument to rapidly change the temperature to drive reaction. RTMA can amplify either DNA or RNA, and can produce either DNA or RNA amplicons, in contrast to most other nucleic acid amplification methods that only produce DNA. RTMA has very rapid kinetics, resulting in a billion-fold amplification within 15-60 minutes. RTMA can be combined with a Hybridization Protection Assay (HPA), which uses a specific oligonucleotide probe labeled with an acridinium ester detector molecule that emits a chemiluminescent signal, for endpoint detection or with molecular torches for real-time detection. There are no wash steps, and no amplicon is ever transferred out of the tube, which simplifies the procedure and reduces the potential for contamination. Thus, the advantages of RTMA include amplification of multiple targets, results can be qualitative or quantitative, no transfers and no wash steps necessary, and detection can be in real time using molecular torches.

As an illustrative embodiment, the RTMA reaction is initiated by treating an RNA target sequence in a nucleic acid sample with both a tagged amplification oligomer and, optionally a blocking oligomer. The tagged amplification oligomer includes a target hybridizing region that hybridizes to a 3′-end of the target sequence and a tag region situated 5′ to the target hybridizing region. The blocking oligomer hybridizes to a target nucleic acid containing the target sequence in the vicinity of the 5′-end of the target sequence. Thus, the target nucleic acid forms a stable complex with the tagged amplification oligomer at the 3′-end of the target sequence and the terminating oligonucleotide located adjacent to or near the determined 5′-end of the target sequence prior to initiating a primer extension reaction. Unhybridized tagged amplification oligomers are then made unavailable for hybridization to a target sequence prior to initiating a primer extension reaction with the tagged priming oligonucleotide, preferably by inactivating and/or removing the unhybridized tagged priming oligonucleotide from the nucleic acid sample. Unhybridized tagged amplification oligomer that has been inactivated or removed from the system is then unavailable for unwanted hybridization to contaminating nucleic acids. In one example of removing unhybridized tagged amplification oligomer from a reaction mixture, the tagged amplification oligomer is hybridized to the target nucleic acid, and the tagged amplification oligomer:target nucleic acid complex is removed from the unhybridized tagged amplification oligomer using a wash step. In this example, the tagged amplification oligomer:target nucleic acid complex may be further complexed to a target capture oligomer and a solid support. In one example of inactivating the unhybridized tagged amplification oligomer, the tagged amplification oligomers further comprise a target-closing region. In this example, the target hybridizing region of the tagged amplification oligomer hybridizes to target nucleic acid under a first set of conditions (e.g., stringency). Following the formation of the tagged amplification oligomer:target nucleic acid complex the unhybridized tagged amplification oligomer is inactivated under a second set of the conditions, thereby hybridizing the target closing region to the target hybridizing region of the unhybridized tagged amplification oligomer. The inactivated tagged amplification oligomer is then unavailable for hybridizing to contaminating nucleic acids. A wash step may also be included to remove the inactivated tagged amplification oligomers from the assay.

An extension reaction is then initiated from the 3′-end of the tagged amplification oligomer with a DNA polymerase, e.g., reverse transcriptase, to produce an initial amplification product that includes the tag sequence. The initial amplification product is then separated from the target sequence using an enzyme that selectively degrades the target sequence (e.g., RNAse H activity). Next, the initial amplification product is treated with a promoter-based oligomer having a target hybridizing region and an RNA polymerase promoter region situated 5′ to the target hybridizing region, thereby forming a promoter-based oligomer:initial amplification product hybrid. The promoter-based oligomer may be modified to prevent the initiation of DNA synthesis, preferably by situating a blocking moiety at the 3′-end of the promoter-based oligomer (e.g., nucleotide sequence having a 3′-to-5′ orientation). The 3′-end of the initial amplification product is then extended to add a sequence complementary to the promoter, resulting in the formation of a double-stranded promoter sequence. Multiple copies of a RNA product complementary to at least a portion of the initial amplification product are then transcribed using an RNA polymerase, which recognizes the double-stranded promoter and initiates transcription therefrom. As a result, the nucleotide sequence of the RNA product is substantially identical to the nucleotide sequence of the target nucleic acid and to the complement of the nucleotide sequence of the tag sequence.

The RNA products may then be treated with a tag-targeting oligomer, which hybridizes to the complement of the tag sequence to form a tag-targeting oligomer: RNA product hybrid, and the 3′-end of the tag-targeting oligomer is extended with the DNA polymerase to produce an amplification product complementary to the RNA product. The DNA strand of this amplification product is then separated from the RNA strand of this amplification product using an enzyme that selectively degrades the first RNA product (e.g., RNAse H activity). The DNA strand of the amplification product is treated with the promoter-based oligomer, which hybridizes to the 3′-end of the second DNA primer extension product to form a promoter-based oligomer:amplification product hybrid. The promoter-based oligomer:amplification product hybrid then re-enters the amplification cycle, where transcription is initiated from the double-stranded promoter and the cycle continues, thereby providing amplification product of the target sequence.

Amplification product can then be used in a subsequent assay. One subsequent assay includes nucleic acid detection, preferably nucleic acid probe-based nucleic acid detection. The detection step may be performed using any of a variety of known ways to detect a signal specifically associated with the amplified target sequence, such as by hybridizing the amplification product with a labeled probe and detecting a signal resulting from the labeled probe. The detection step may also provide additional information on the amplified sequence, such as all or a portion of its nucleic acid base sequence. Detection may be performed after the amplification reaction is completed, or may be performed simultaneous with amplifying the target region, e.g., in real time. In one embodiment, the detection step allows detection of the hybridized probe without removal of unhybridized probe from the mixture (see, e.g., U.S. Pat. Nos. 5,639,604 and 5,283,174).

The amplification methods as disclosed herein, in certain embodiments, also preferably employ the use of one or more other types of oligonucleotides that are effective for improving the sensitivity, selectivity, efficiency, etc., of the amplification reaction.

### Target Capture

At times, it may be preferred to purify or enrich a target nucleic acid from a sample prior to nucleic acid amplification. Target capture, in general, refers to capturing a target polynucleotide onto a solid support, such as magnetically attractable particles, wherein the solid support retains the target polynucleotide during one or more washing steps of the target polynucleotide purification procedure. In this way, the target polynucleotide is substantially purified prior to a subsequent nucleic acid amplification step. Many target capture methods are known in the art and suitable for use in conjunction with the methods described herein. For example, any support may be used, e.g., matrices or particles free in solution, which may be made of any of a variety of materials, e.g., nylon, nitrocellulose, glass, polyacrylate, mixed polymers, polystyrene, silane polypropylene, or metal. Illustrative examples use a support that is magnetically attractable particles, e.g., monodisperse paramagnetic beads to which an immobilized probe is joined directly (e.g., via covalent linkage, chelation, or ionic interaction) or indirectly (e.g., via a linker), where the joining is stable during nucleic acid hybridization conditions. In short, essentially any technique available to the skilled artisan may be used provided it is effective for purifying a target nucleic acid sequence of interest prior to amplification.

### Nucleic Acid Detection

Any labeling or detection system or both used to monitor nucleic acid hybridization can be used to detect STEC amplicons. Such systems are well known in the art.

Detection systems typically employ a detection oligonucleotide of one type or another in order to facilitate detection of the target nucleic acid of interest. Detection may either be direct (i.e., probe hybridized directly to the target) or indirect (i.e., a probe hybridized to an intermediate structure that links the probe to the target). A probe's target sequence generally refers to the specific sequence within a larger sequence which the probe hybridizes specifically. A detection probe may include target-specific sequences and other sequences or structures that contribute to the probe's three-dimensional structure, depending on whether the target sequence is present

Essentially any of a number of well known labeling and detection system that can be used for monitoring specific nucleic acid hybridization can be used in conjunction with the present invention. Included among the collection of useful labels are fluorescent moieties (either alone or in combination with “quencher” moieties), chemiluminescent molecules, and redox-active moieties that are amenable to electronic detection methods. In some embodiments, preferred fluorescent labels include non-covalently binding labels (e.g., intercalating dyes) such as ethidium bromide, propidium bromide, chromomycin, acridine orange, and the like.

In some applications, probes exhibiting at least some degree of self-complementarity are desirable to facilitate detection of probe:target duplexes in a test sample without first requiring the removal of unhybridized probe prior to detection. By way of example, structures referred to as “molecular torches” and “molecular beacons” are designed to include distinct regions of self-complementarity and regions of target-complementarity. Molecular torches are fully described in U.S. Pat. Nos. 6,849,412, 6,835,542, 6,534,274, and 6,361,945, and molecular beacons are fully described in U.S. Pat. Nos. 5,118,801, 5,312,728, and 5,925,517.

Synthetic techniques and methods of attaching labels to nucleic acids and detecting labels are well known in the art.

### Immunological-Based Detection Assays

Methods and compositions are provided herein for the immunological detection of E. coli adulterants in a sample using ecf and wzx and/or stx. Such methods include enzyme-linked immunoabsorbent assays (ELISA) which is a widely used for the detection of E. coli. In the present system, antibodies (e.g., monoclonal or polyclonal or fragments thereof) are generated against an ecf, wzx, and/or stx (stx1 and stx2) polypeptide according to well established methods known in the art. Test devices for immunological assays include conventional microtitre plates, dipsticks, immunofiltration, and capillary migration assays. Such systems are also useful as visual tests. Immunological detection systems utilizing antibodies having specificity to an ecf, wzx, or stx polypeptide are useful for simple, fast, and high-volume screening methods, with the identification of negative and positive samples in a short time period. According to the methods, detection of ecf and wzx is taken as an indication of the presence of E. coli O157:H7; detection of ecf and the absence of wzx is taken as an indication of the presence of non-O157:H7 shiga toxin (stx)-containing E. coli (STEC); and detection of ecf and stx is taken as an indication of the presence of enterohemorrhagic Escherichia coli (EHEC).

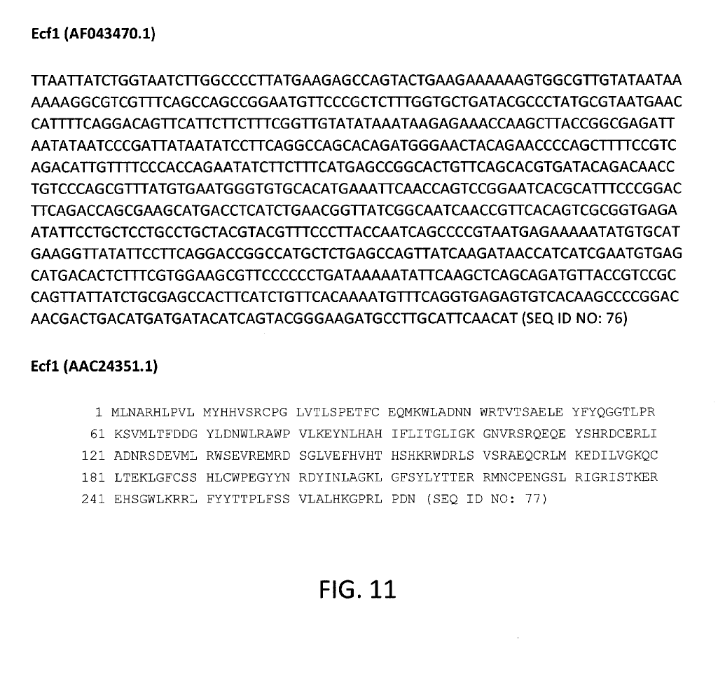

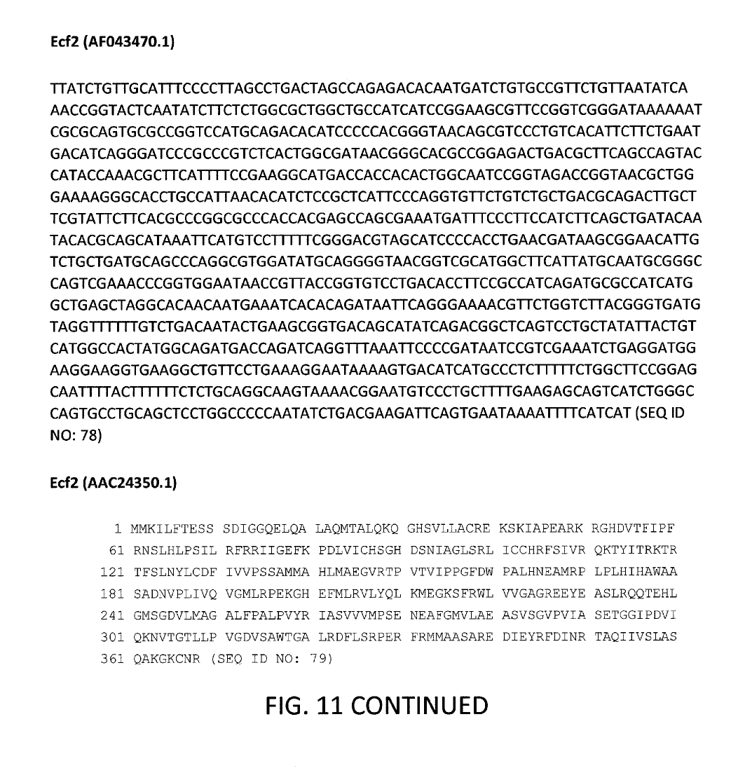

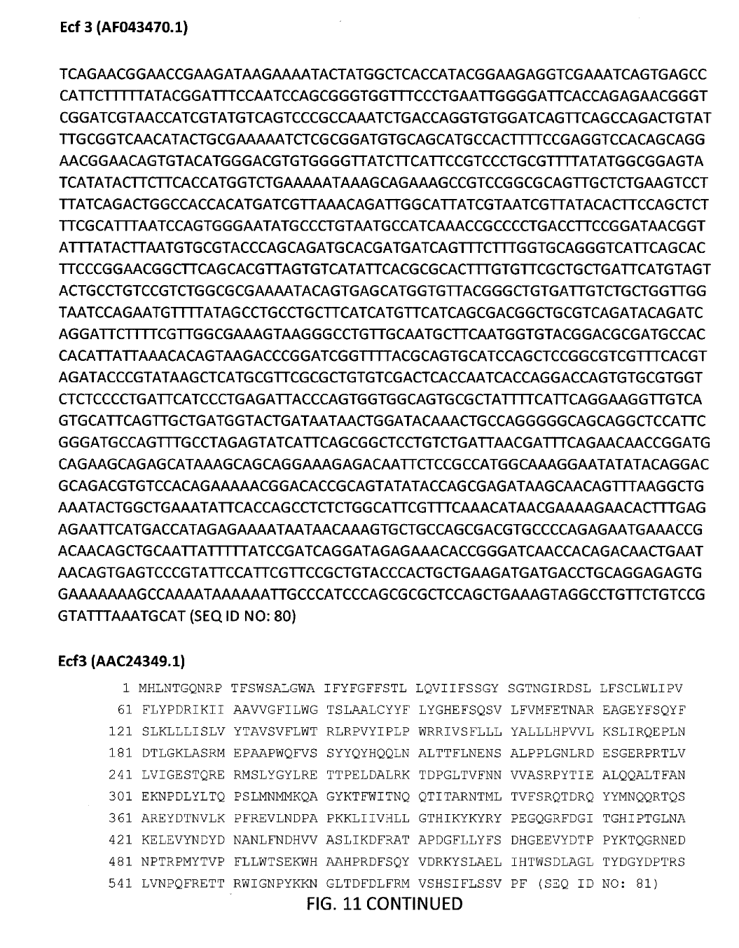

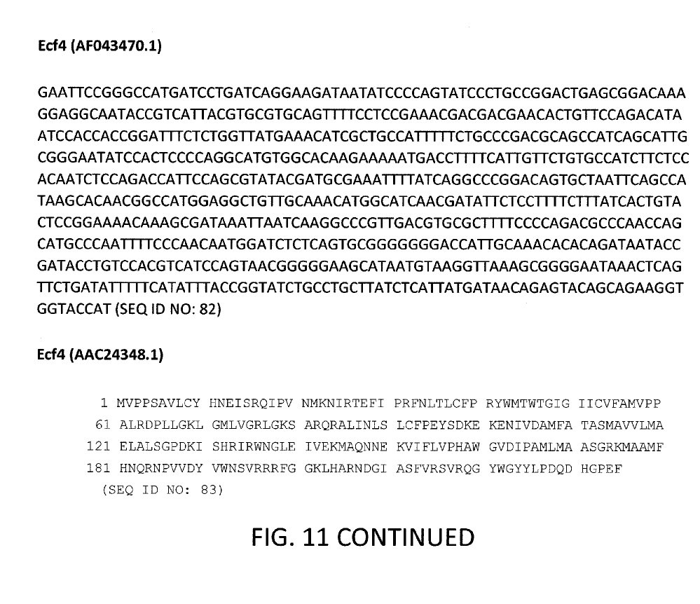

As is disclosed herein, the pO157 ecf (E. coli attaching and effacing [eae] gene-positive conserved fragments) operon is especially useful in the disclosed methods. This operon encodes four genes as one operon: ecf1, ecf2, ecf3, and ecf4. These ecf genes are involved in bacterial cell wall synthesis encoding bacterial surface structure-associated proteins. Both ecf1 and ecf2 respectively encode a polysaccharide deacetylase and a lipopolysaccharide (LPS) α-1,7-N-acetylglucosamine transferase (also designated WabB). ecf3 encodes an outer membrane protein associated with bacterial invasion. And ecf4 encodes a second LPS—lipid A myristoyl transferase. Exemplary Ecf polypeptides (Ecf1, Ecf2, Ecf3, and Ecf 4) are described in FIG. 11 as well as in Table 5 (in connection with Genbank accession number). Other Ecf polypeptides useful in the invention are those having identity with those described in FIG. 11. Such sequence identity is typically 90%, 92% or 95% or greater between an Ecf polypeptide described herein and a polypeptide used for comparative purposes. To determine the percent identity of two polypeptides standard methods well known in the art are employed. Fragments of Ecf polypeptides are also useful in the invention.

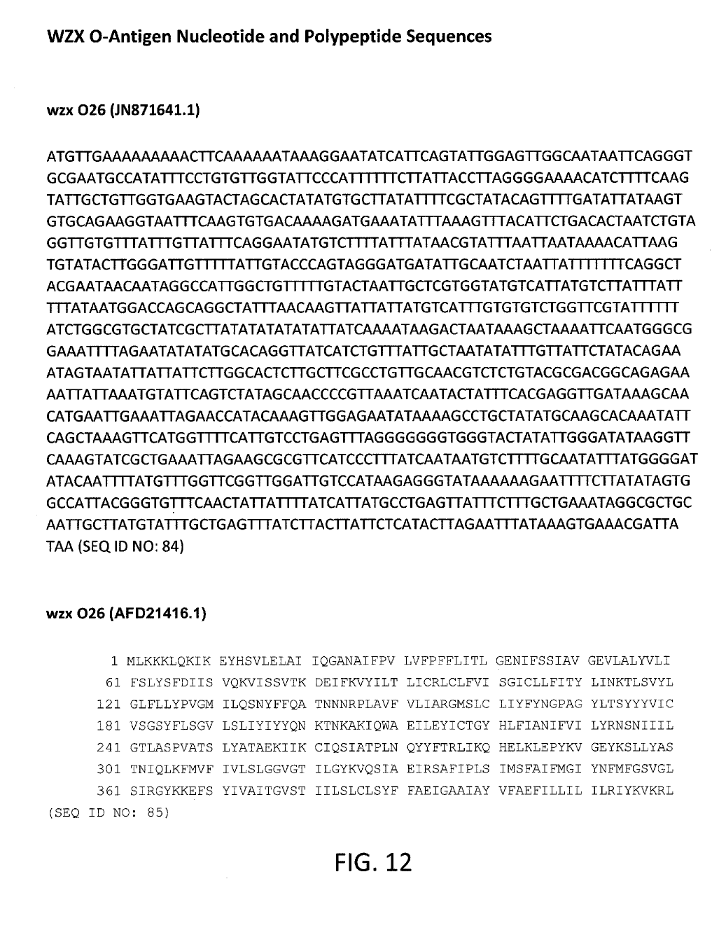

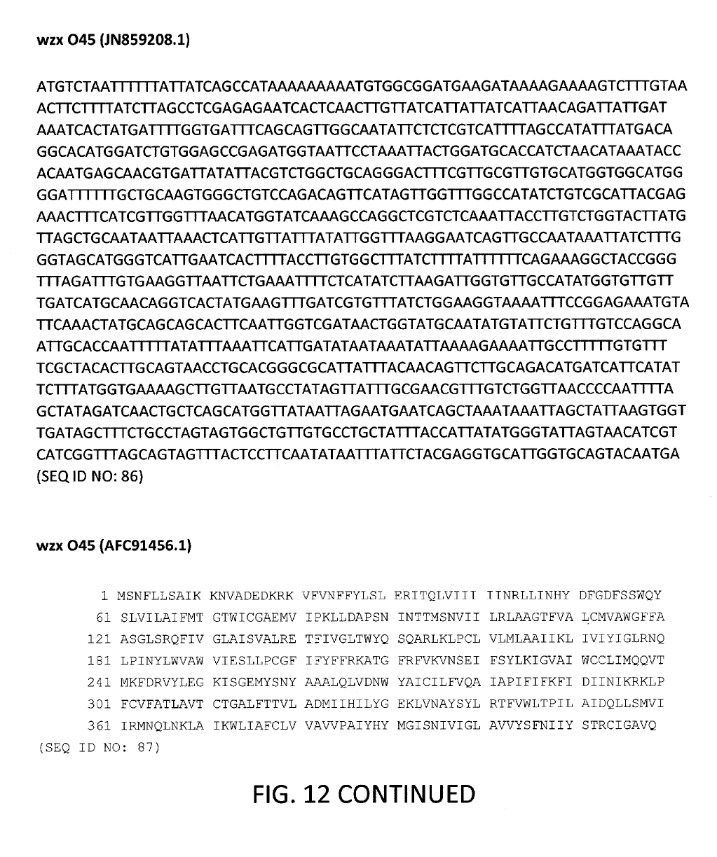

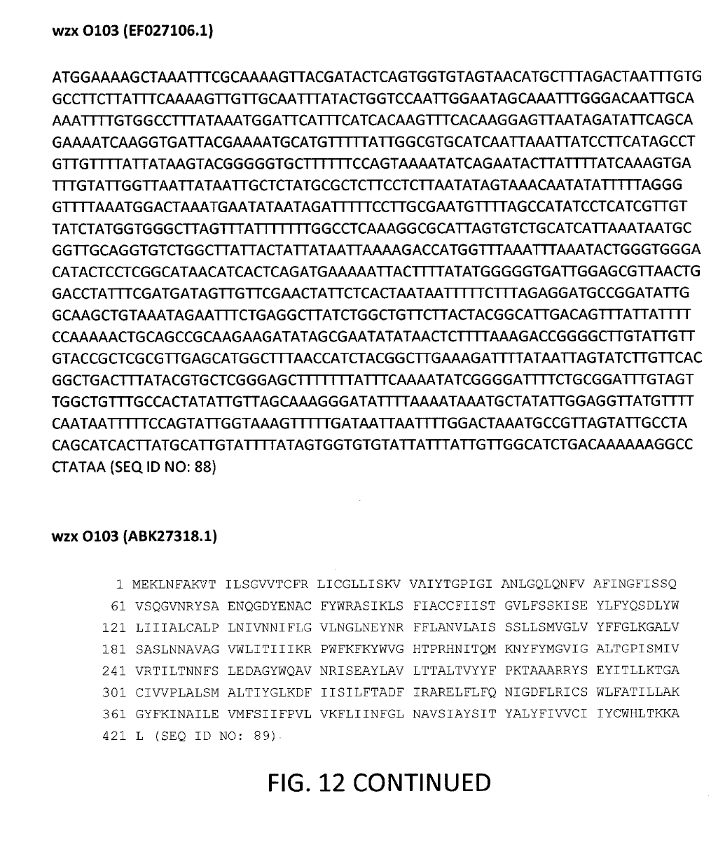

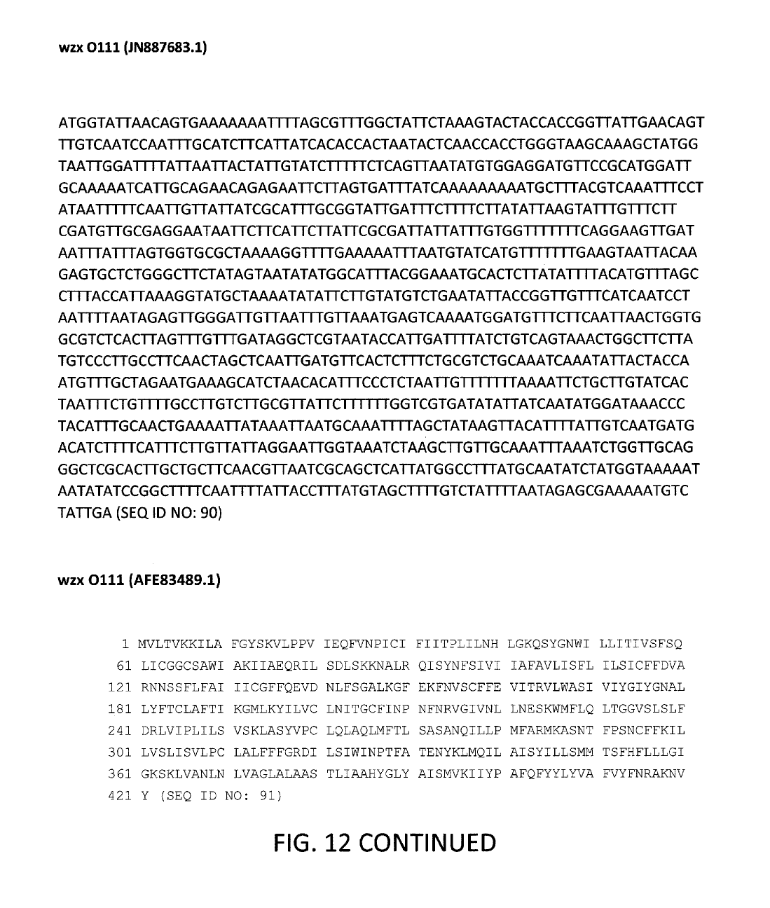

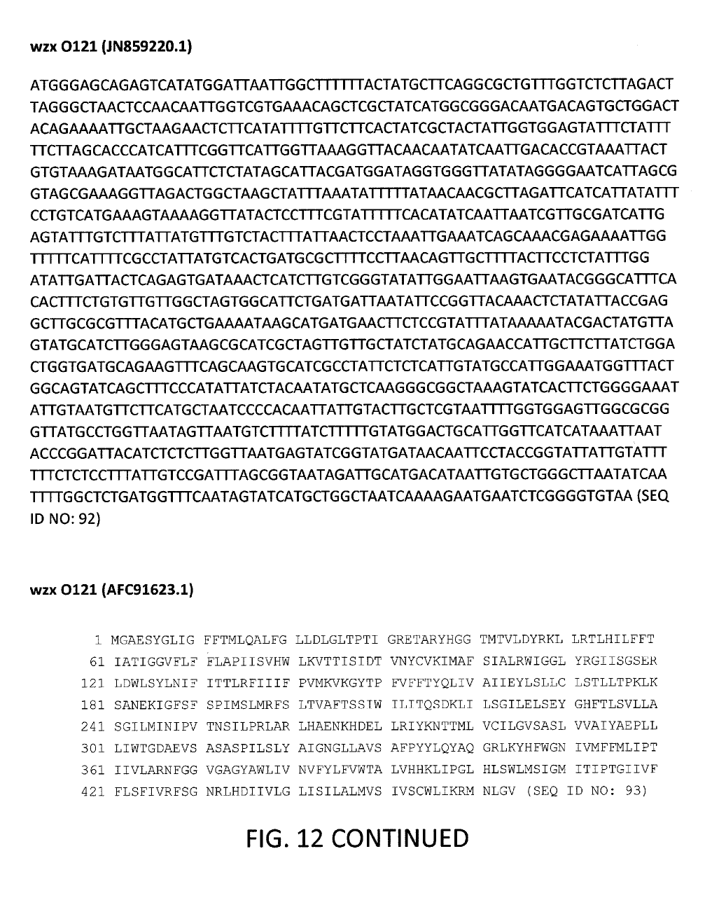

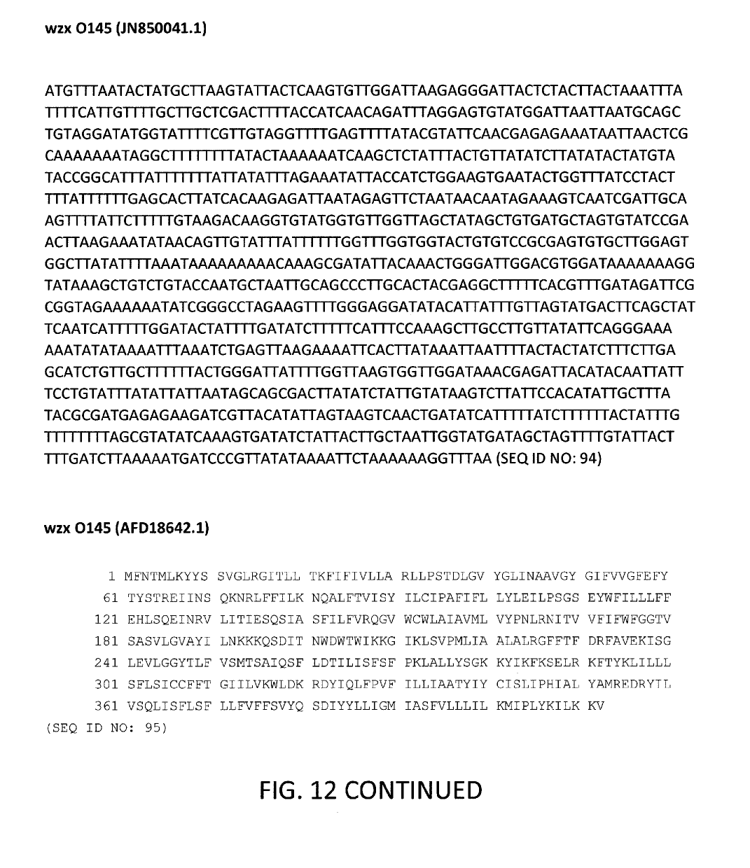

As is further disclosed herein, detection of wzx is especially useful in the methods explained herein. Wzx is an E. coli translocase. Exemplary wzx polypeptides are described in FIG. 12 as well as in Table 5 (in connection with Genbank accession number). Other wzx polypeptides useful in the invention are those having identity with those described in FIG. 12. Such sequence identity is typically 90%, 92% or 95% or greater between a wzx polypeptide described herein and a polypeptide used for comparative purposes. To determine the percent identity of two polypeptides standard methods well known in the art are employed. Fragments of wzx polypeptides are also useful in the invention.

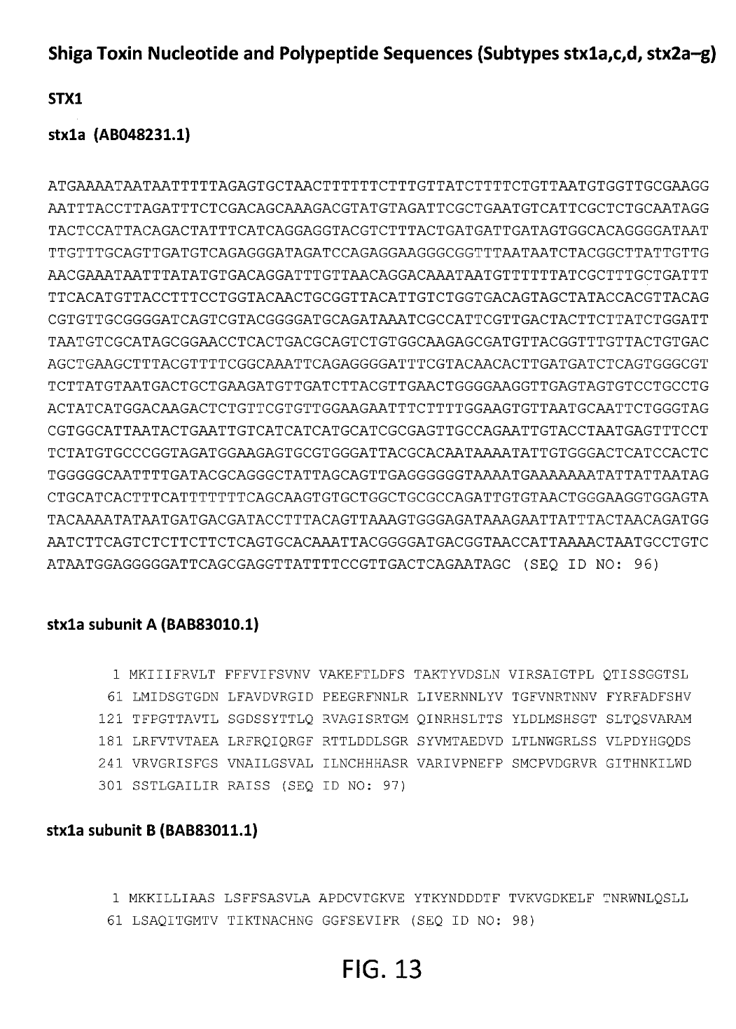

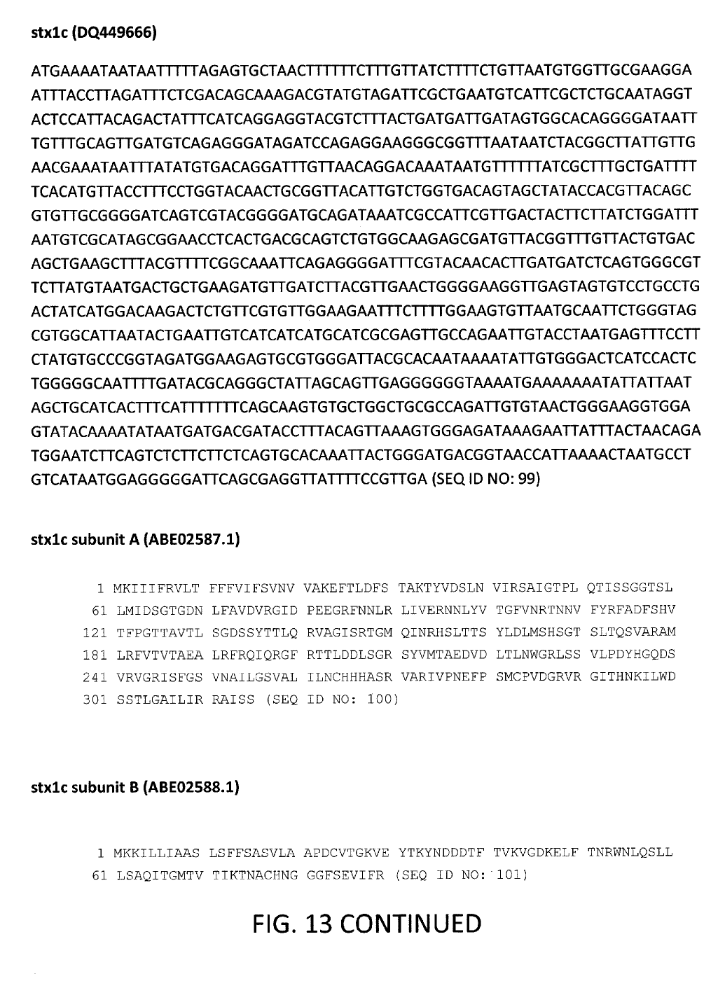

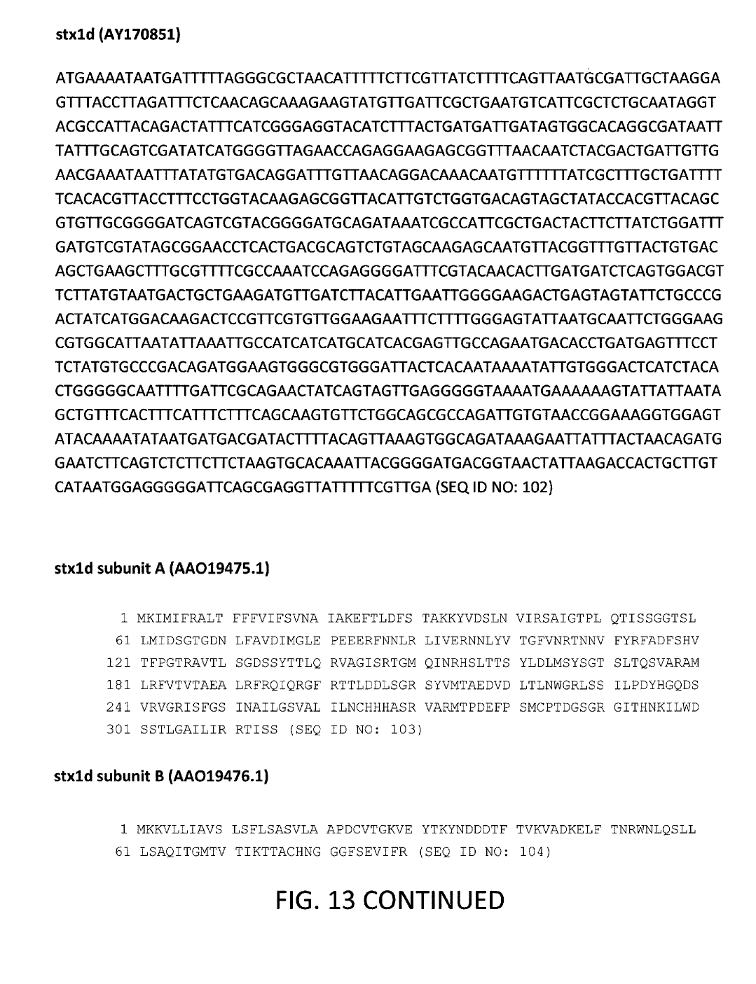

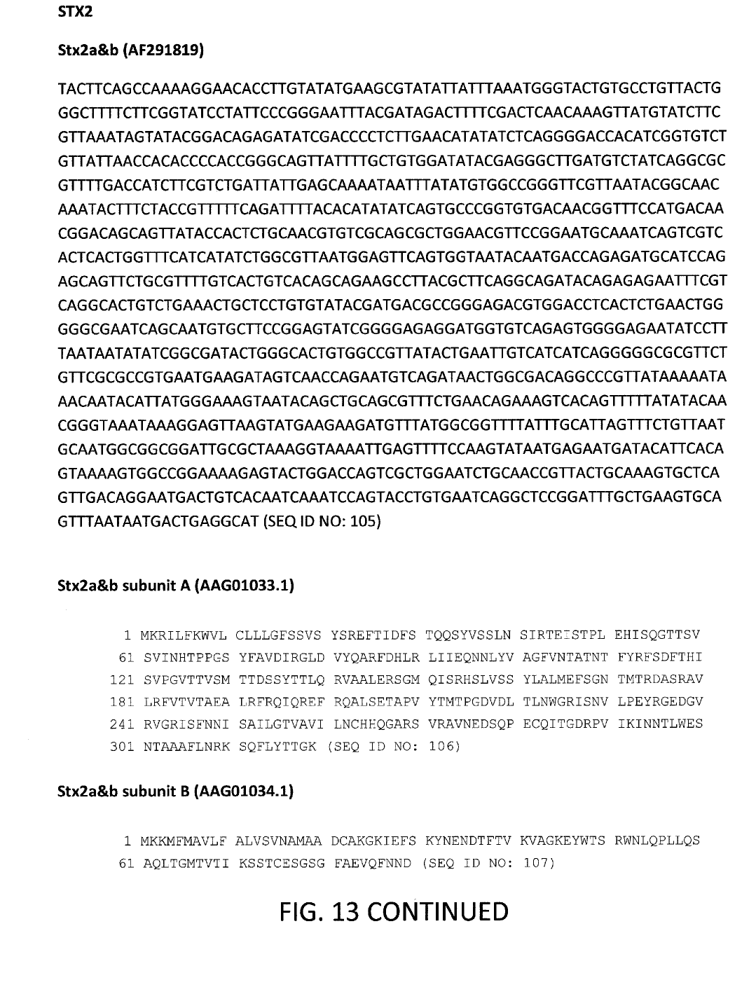

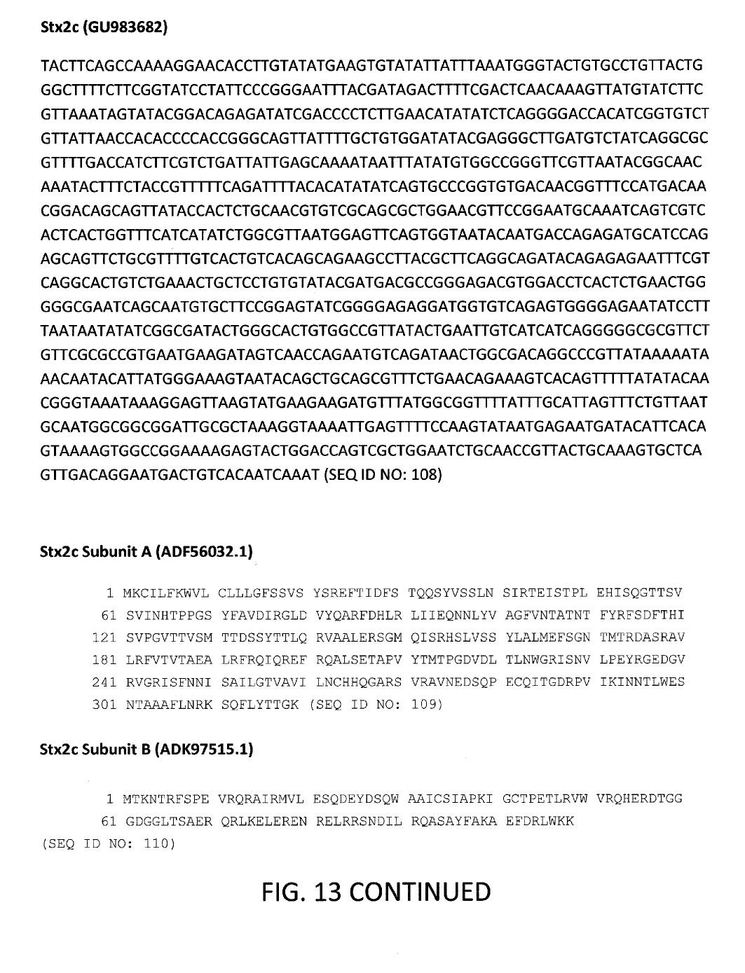

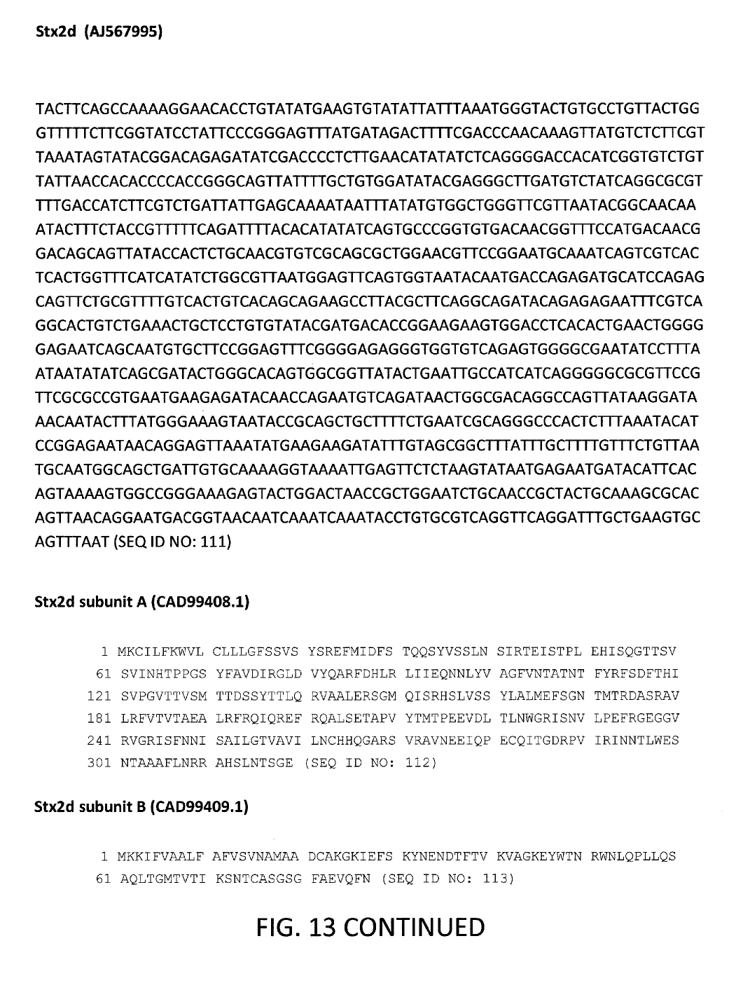

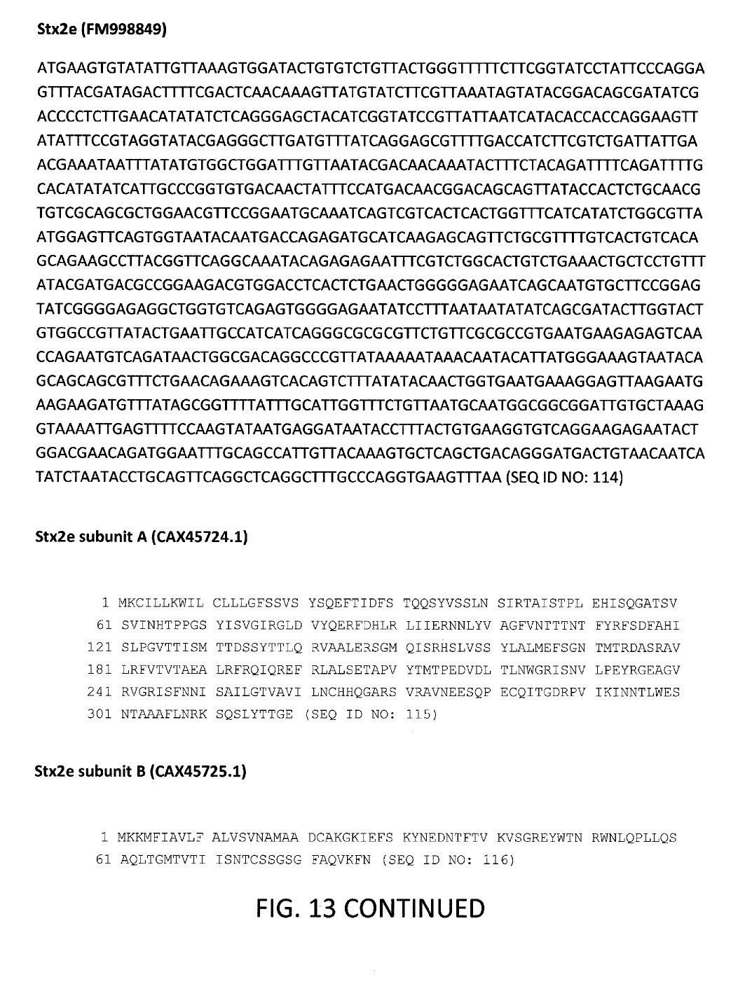

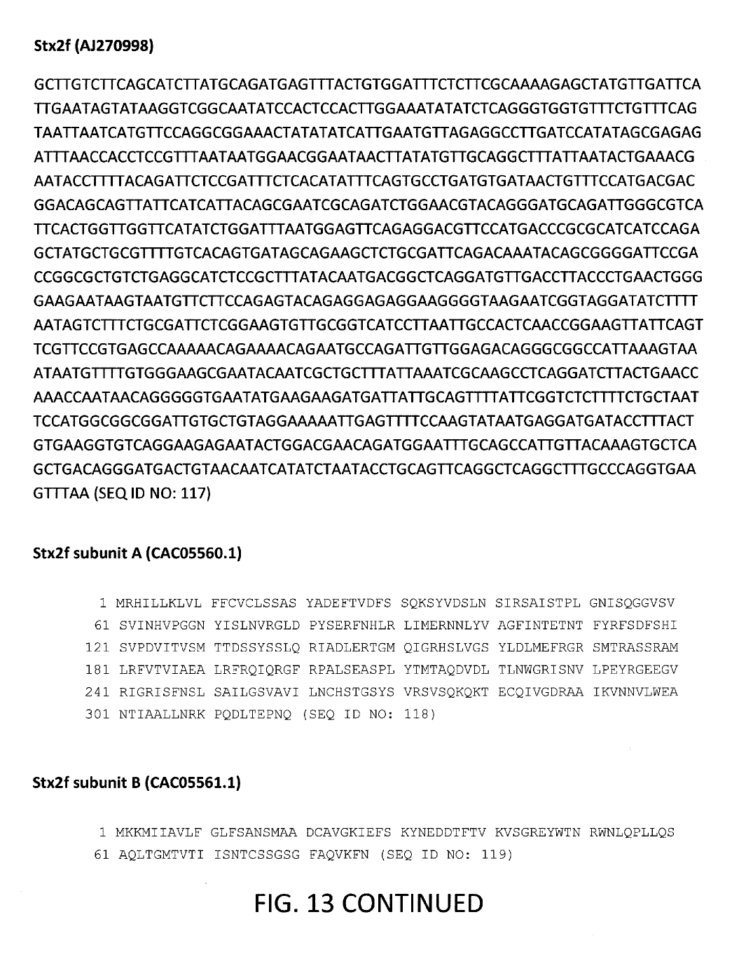

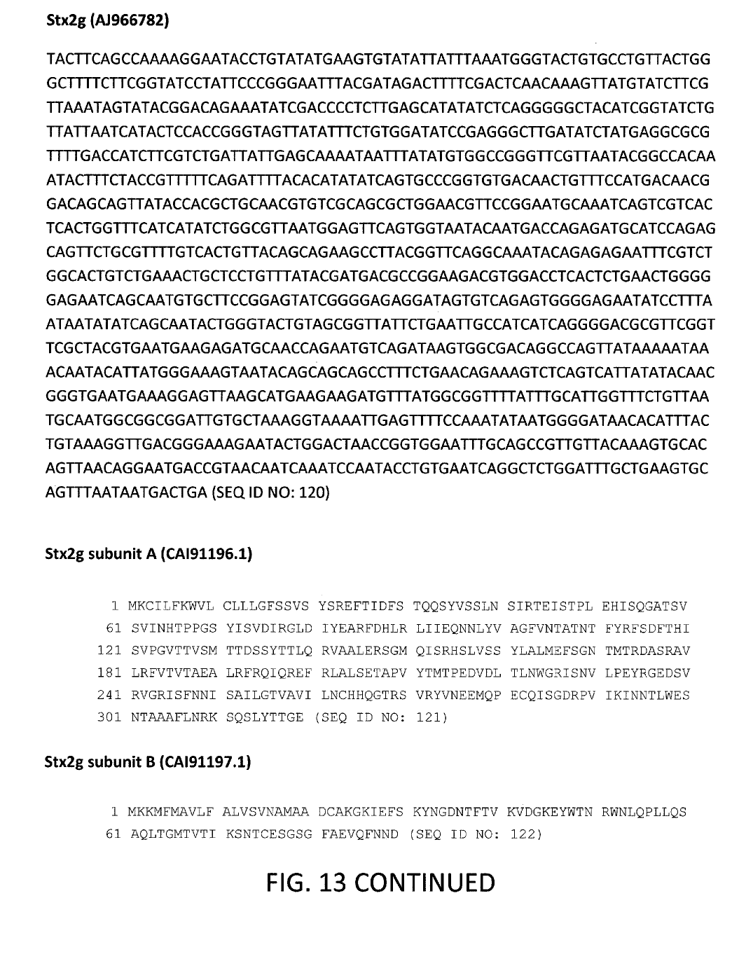

And detection of stx (E. coli shiga-like toxins, e.g., stx1 and stx2) is especially useful in the disclosed methods. Exemplary stx polypeptides are described in FIG. 13 as well as in Table 5 (in connection with Genbank accession number). Other stx polypeptides useful in the invention are those having identity with those described in FIG. 13. Such sequence identity is typically 90%, 92% or 95% or greater between a stx polypeptide described herein and a polypeptide used for comparative purposes. To determine the percent identity of two polypeptides standard methods well known in the art are employed. Fragments of stx polypeptides are also useful in the invention.

### Meat, Produce, and Other Products and Foodstuffs

The methods and compositions described herein are useful for producing a packaged lot of meat or produce including foodstuffs free of a pathogenic E. coli adulterant. Typically, samples of lots of meat product (e.g., a lot of meat such as raw ground beef, beef trim, high fat ground beef, and raw ground beef components for example, beef and veal bulk packed manufacturing trimmings and other beef and veal components such as primal cuts, sub primal cuts, head meat, cheek meat, esophagus meat, heart, and advanced meat recovery product intended for grinding) or produce (e.g. fruits or vegetables such as leafy green vegetables including lettuce and spinach) are processed according to standard methods known in the art for testing. Such processing may include a step for enriching for the presence of an E. coli adulterant from the lot of meat or produce. Analysis of the sample includes one or more of the nucleic acid or polypeptide detection assays described herein. If desired, multiple samples may be tested. The sample is then subject to a hybridization assay or to an immunological assay or both as described herein to test for the presence of (i) ecf and (ii) wzx and/or stx. Following testing and analysis, results indicative of the absence of these markers is taken as an indication that the lot of meat or produce is free of an E. coli adulterant and may be packaged as a product. Methods for packaging meat and produce are well known and typically include the use of cartons, containers, plastic wrap, or trays wrapped with plastic. Packaged meat and produce products free of pathogenic E. coli may be subsequently shipped to a destination for sale or consumption. Shipping typically involves transport of the product from a processor to a distribution center or directly to a grocery store or restaurant or other consumer of the product. These methods and compositions are also useful for producing other products free of E. coli contamination. Examples include unpasteurized fresh-pressed juices such as apple cider, yogurt, and cheese made from raw milk.

### Kits

The invention also features a kit for carrying out the described methods according to the present invention described herein. The kit includes nucleic acid probes or primers that may be labeled, reagents and containers for carrying out the hybridization assay, positive and negative control reagents, and instructions for performing the assay. Oligonucleotides, probes, and primers are readily designed nucleic acids known in the art for the ecf operon, wzx, and stx (stx1 and stx2). Exemplary sequences are shown in FIGS. 11-13 as well as in Table 5.

Kits may also include antibodies specific for any of the polypeptides or fragments thereof disclosed herein and appropriate reagents for an immunological-based assay for detecting an ecf, wzx, and stx polypeptide.

Some kits contain at least one target capture oligomer for hybridizing to a target nucleic acid. Some kits for detecting the presence or abundance of two or more target nucleic acids contain two or more target capture oligomers each configured to selectively hybridize to each of their respective target nucleic acids.

Some kits contain at least one first amplification oligomer for hybridizing to a target nucleic acid. Some kits for detecting the presence or abundance of two or more target nucleic acids contain two or more first amplification oligomers, each configured to selectively hybridize to their respective target nucleic acids.

Some kits contain chemical compounds used in performing the methods herein, such as enzymes, substrates, acids or bases to adjust pH of a mixture, salts, buffers, chelating agents, denaturants, sample preparation agents, sample storage or transport medium, cellular lysing agents, total RNA isolation components and reagents, partial generalized RNA isolation components and reagents, solid supports, and other inorganic or organic compounds. Kits may include any combination of the herein mentioned components and other components not mentioned herein. Components of the kits can be packaged in combination with each other, either as a mixture or in individual containers. It will be clear to skilled artisans that the invention includes many different kit configurations.

The kits of the invention may further include additional optional components useful for practicing the methods disclosed herein. Exemplary additional components include chemical-resistant disposal bags, tubes, diluent, gloves, scissors, marking pens, and eye protection.

### Example 1

We have developed a PCR to simultaneously detect E. coli O157:H7 and non-O157:H7 STEC, which provides sensitivity to identify non-O157:H7 STEC such as the big six virulent, non-O157:H7.

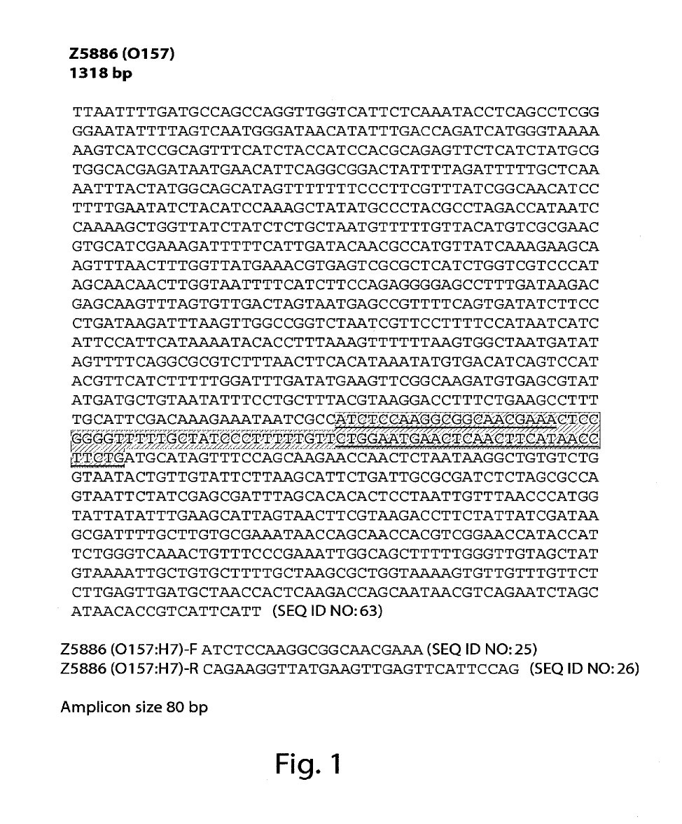

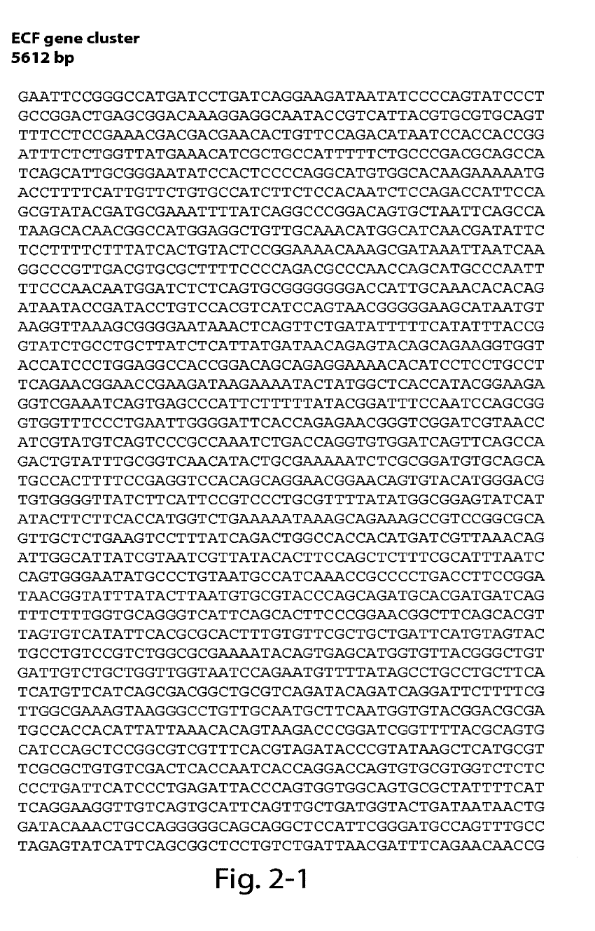

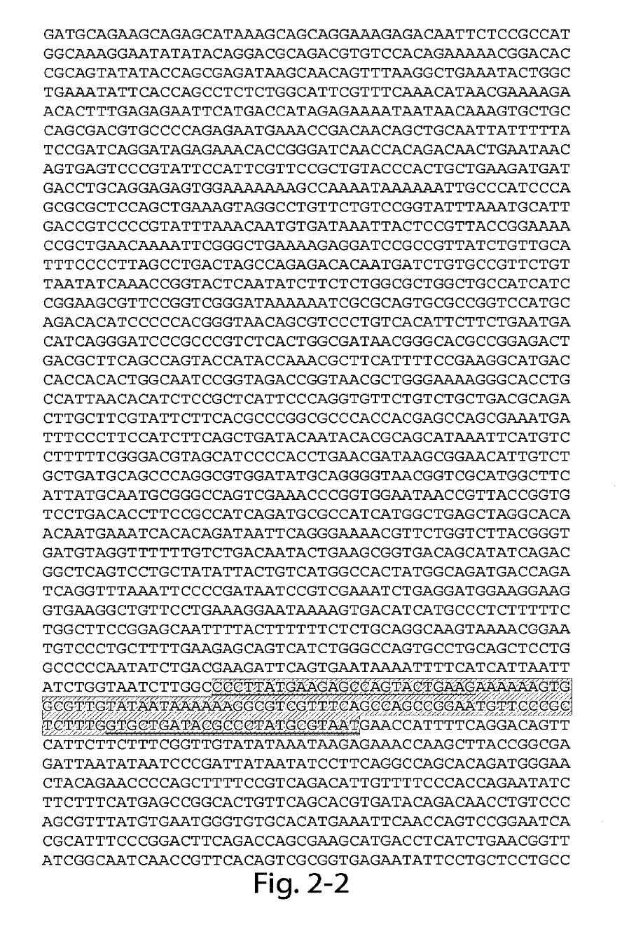

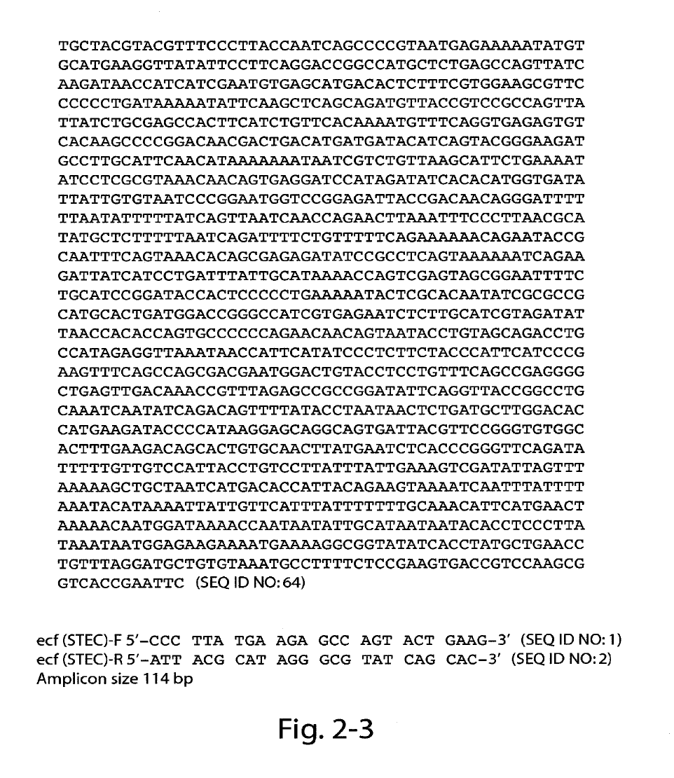

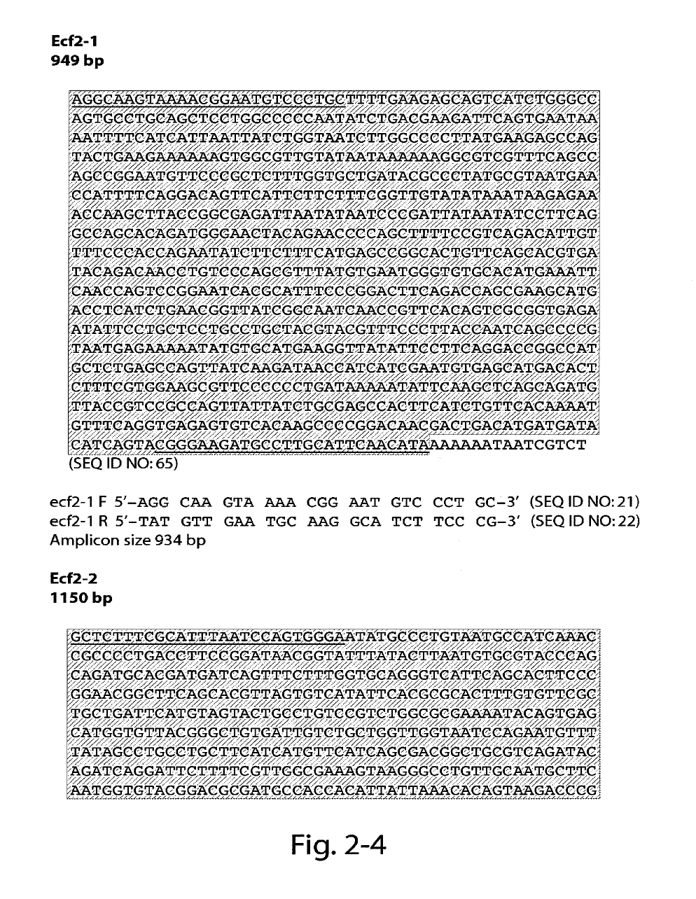

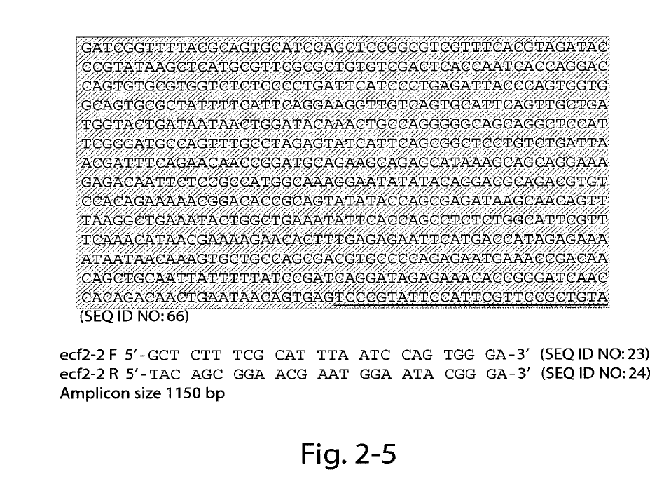

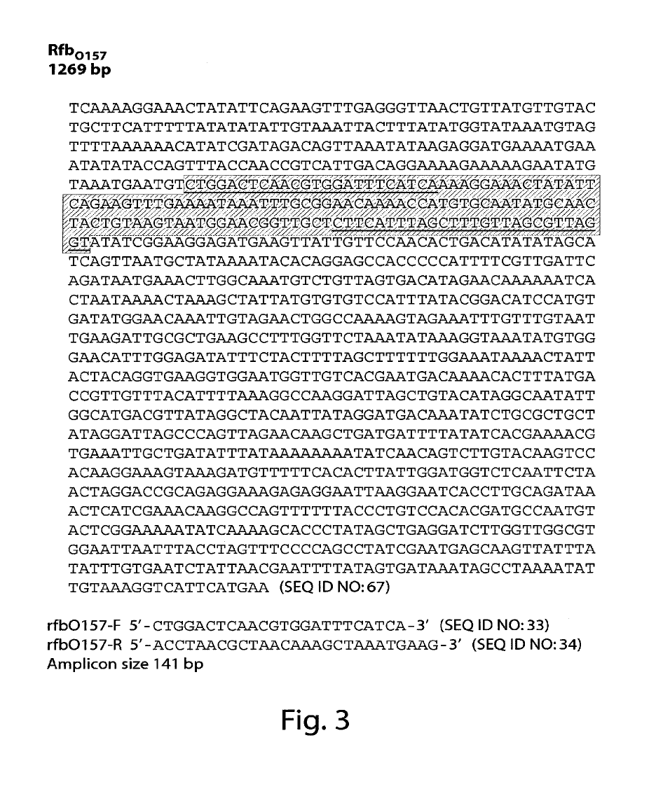

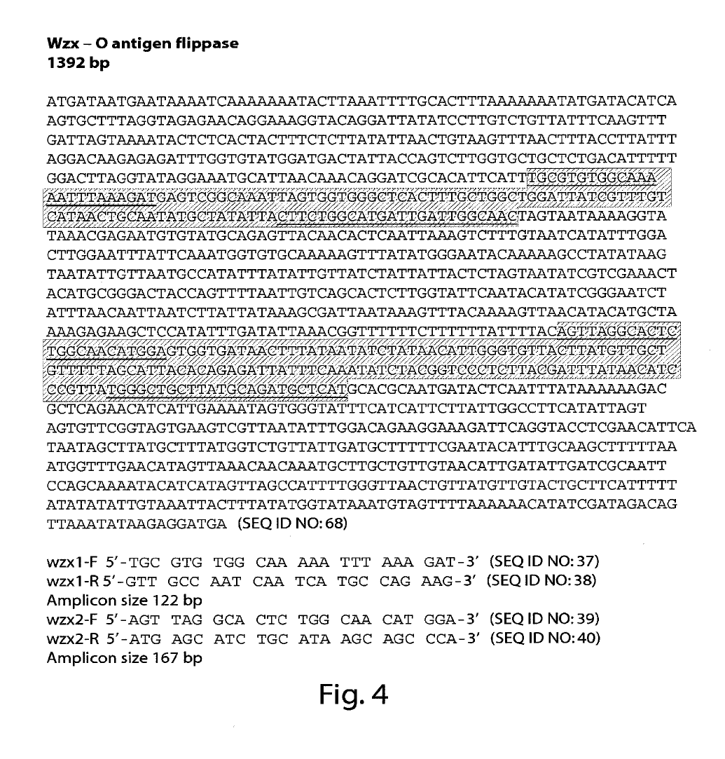

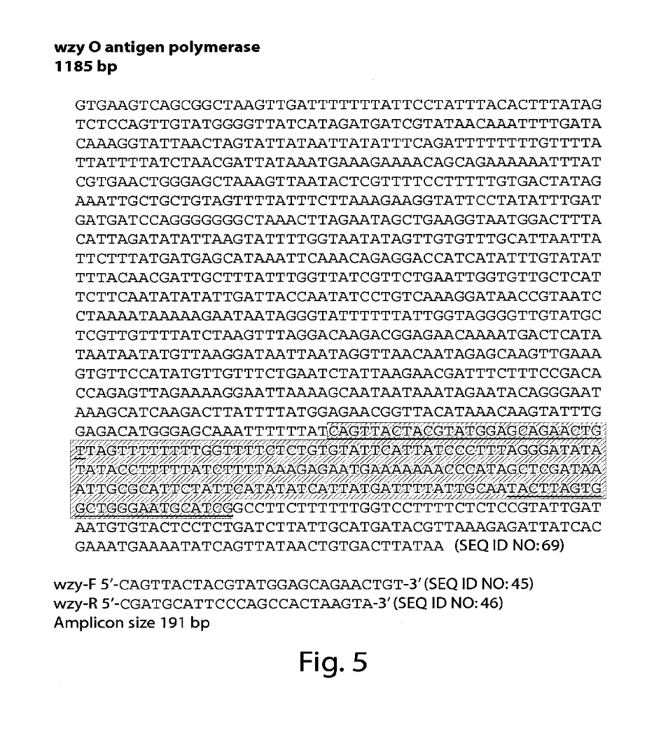

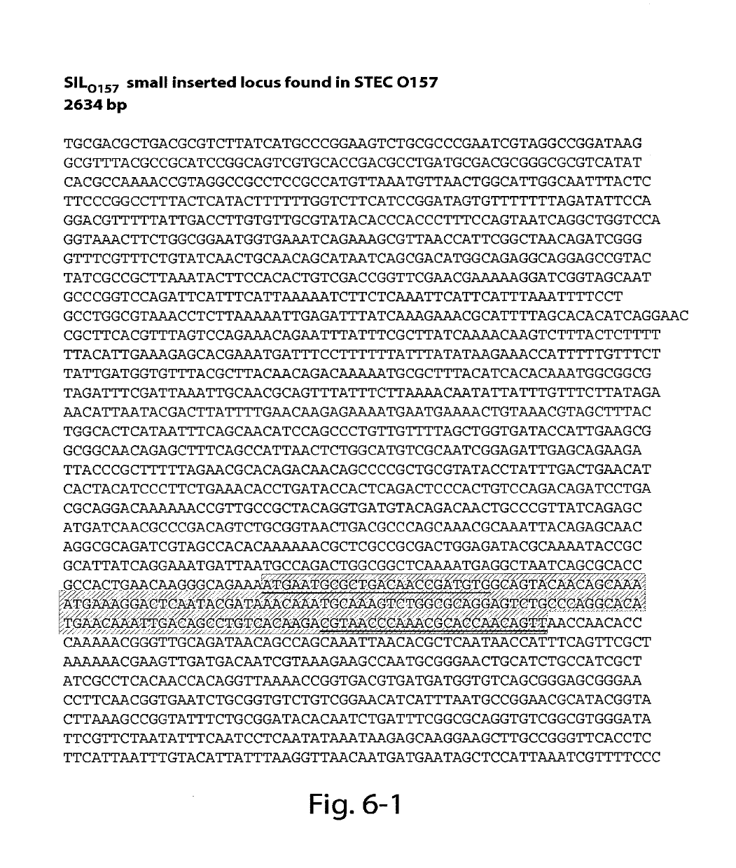

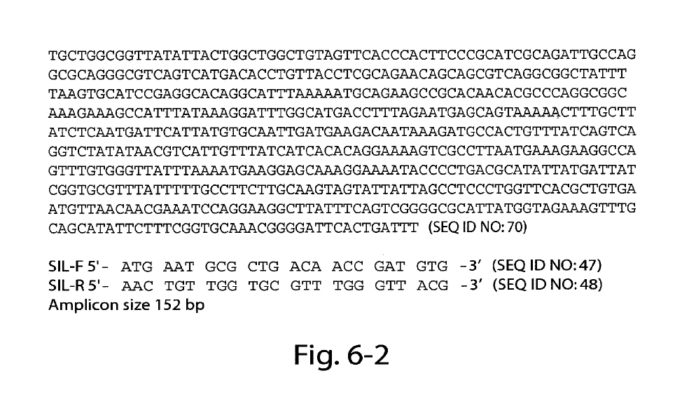

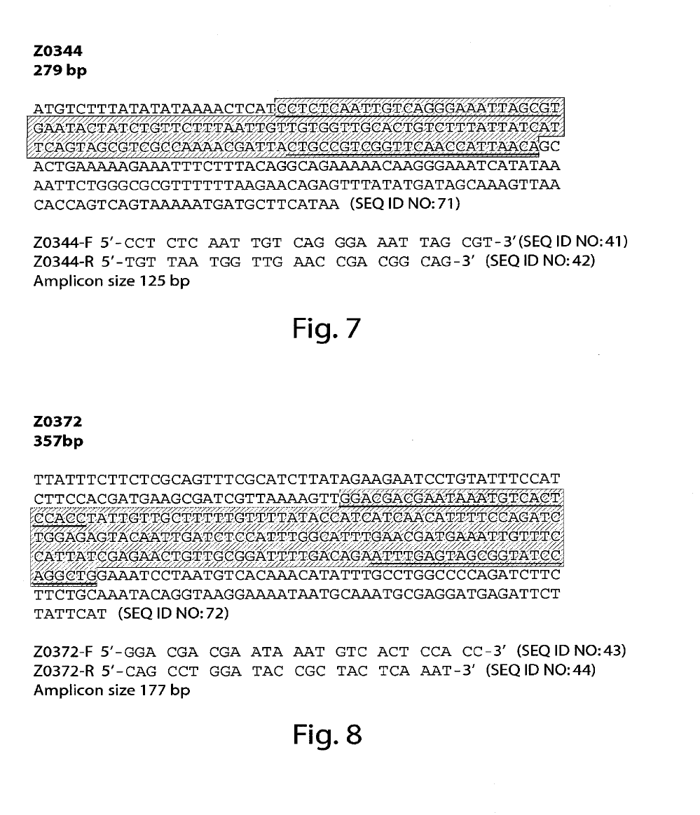

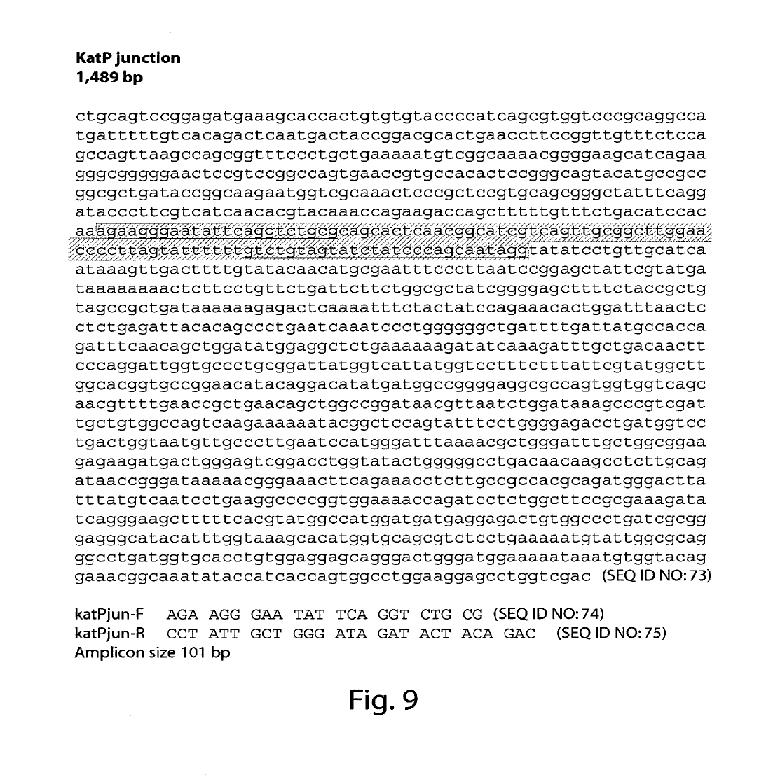

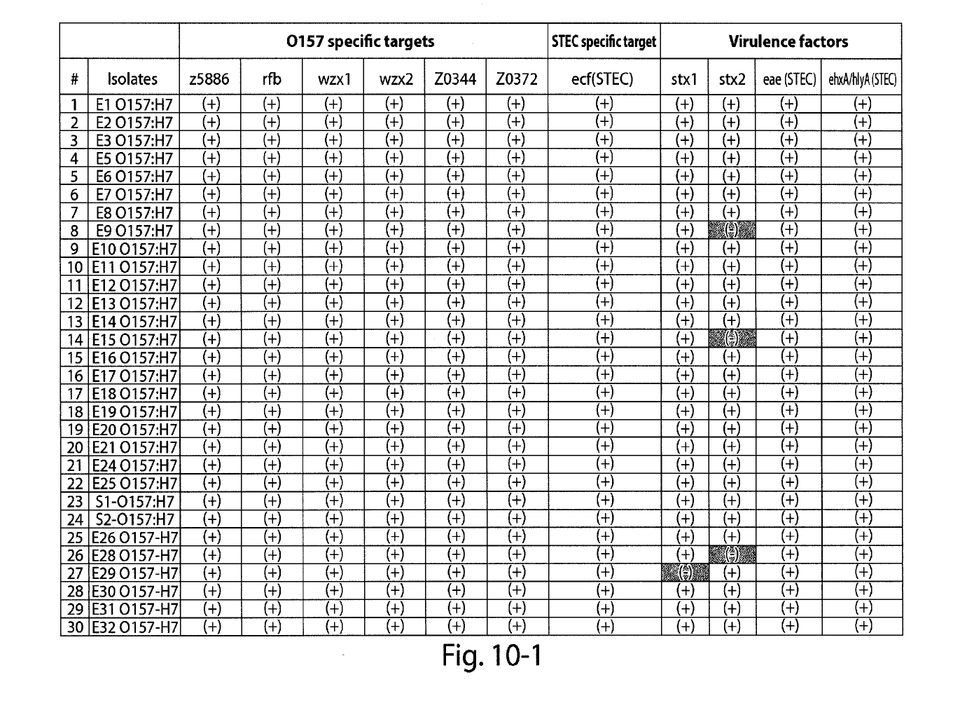

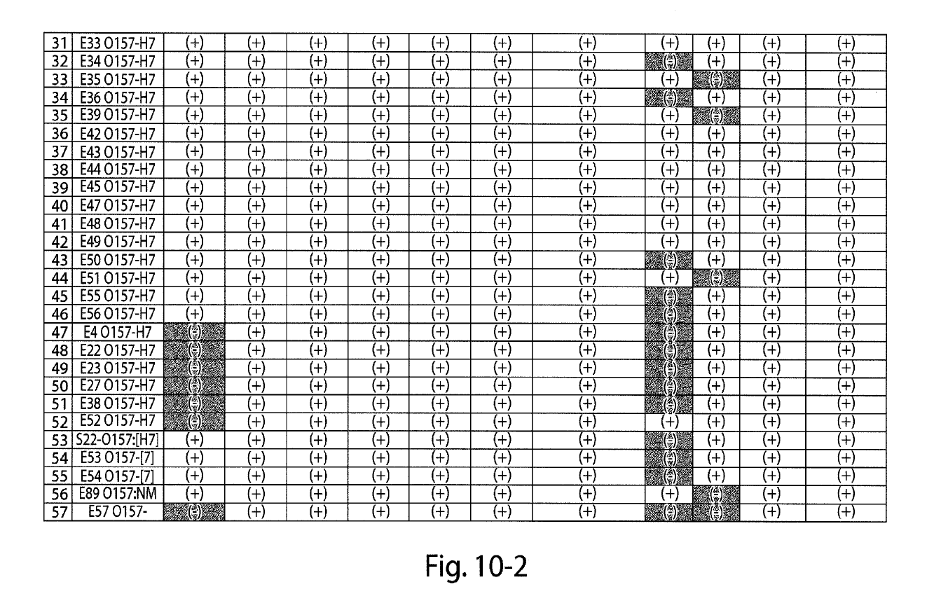

Useful targets identified for such assays include those found in FIGS. 1-9. Useful oligonucleotides for amplifying such targets are found in FIGS. 1-9 as well.

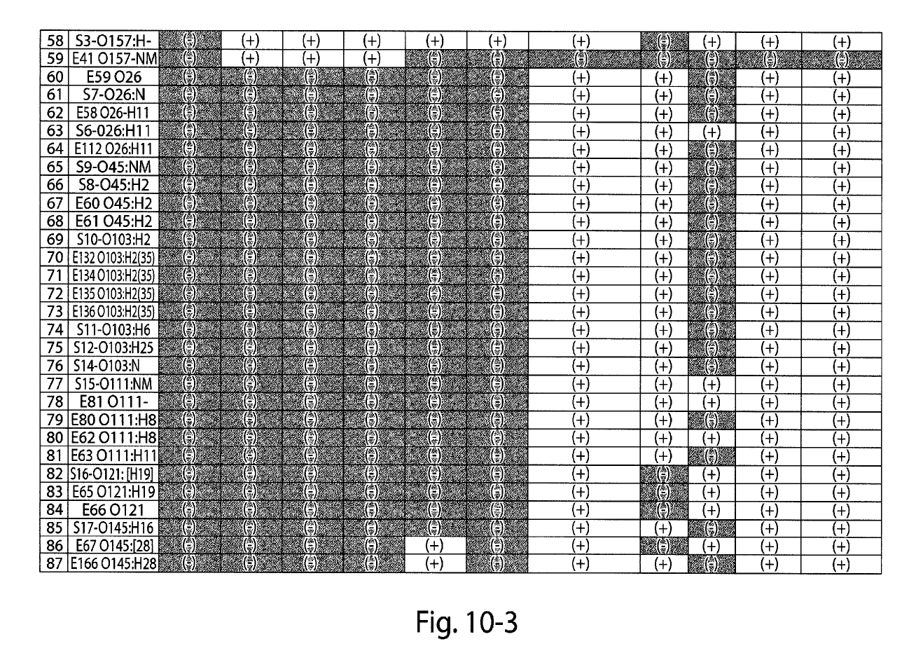

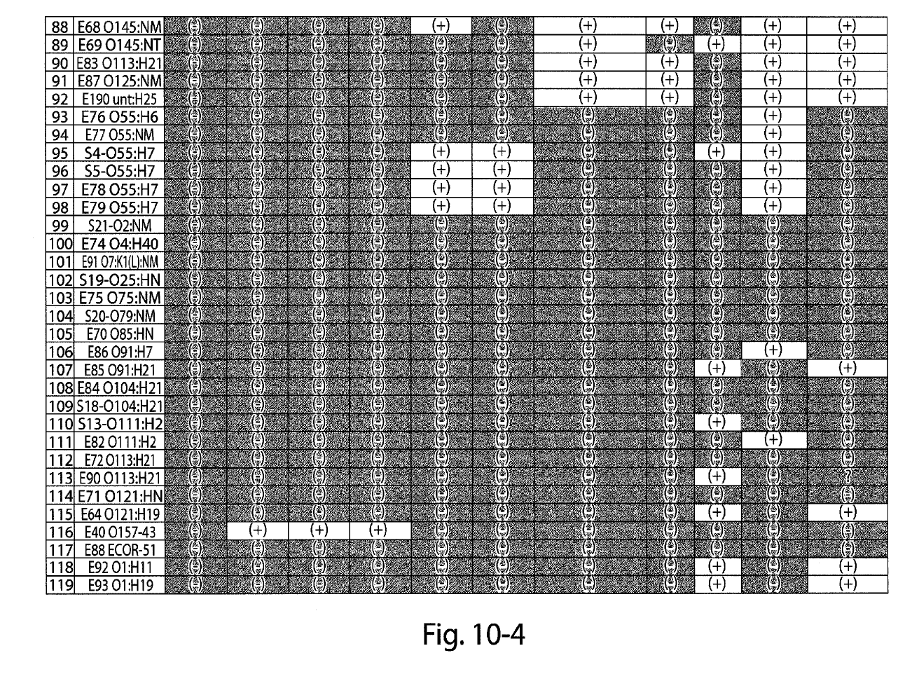

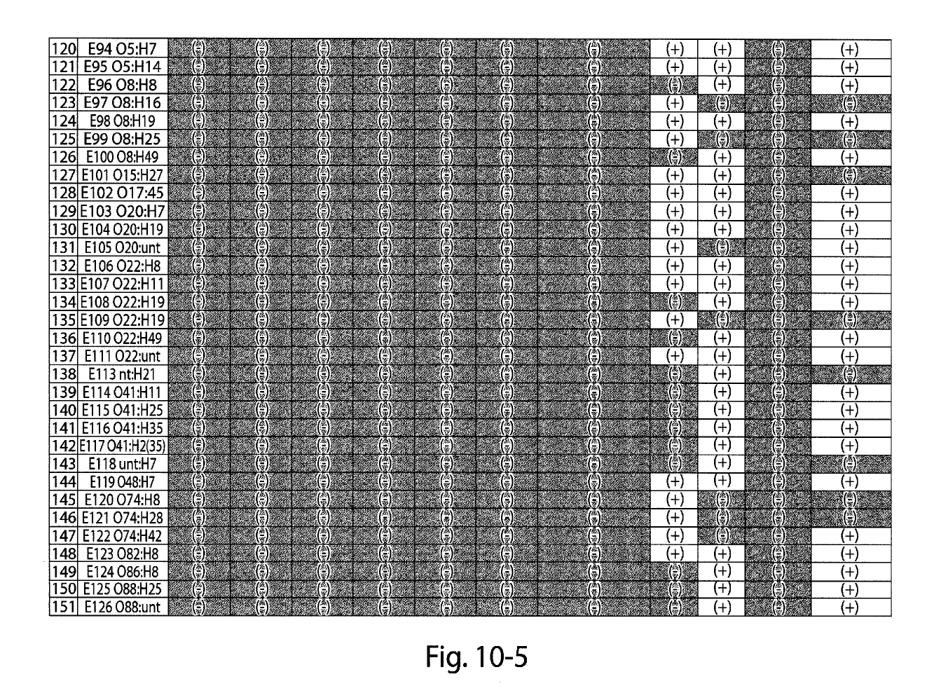

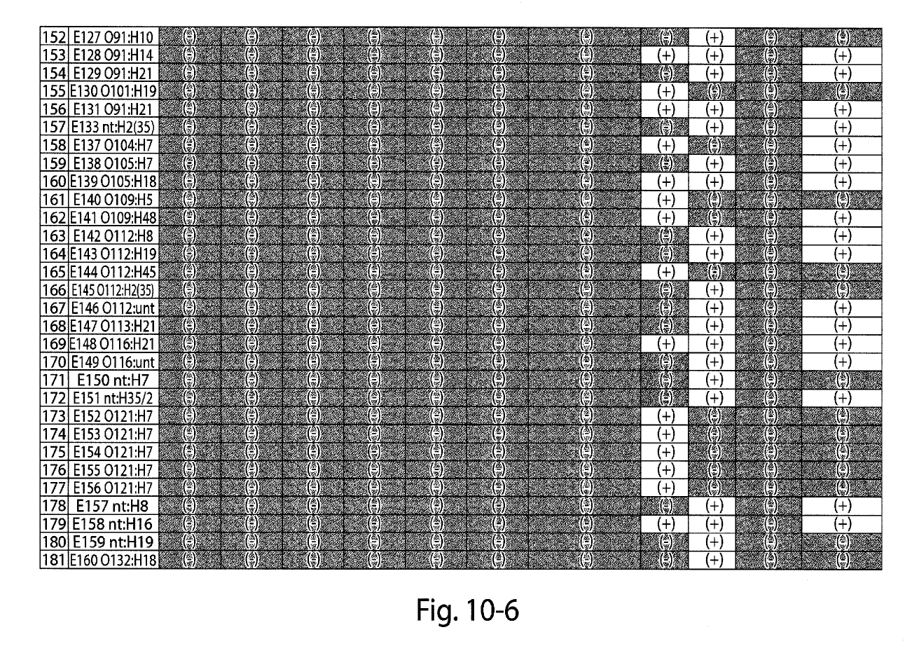

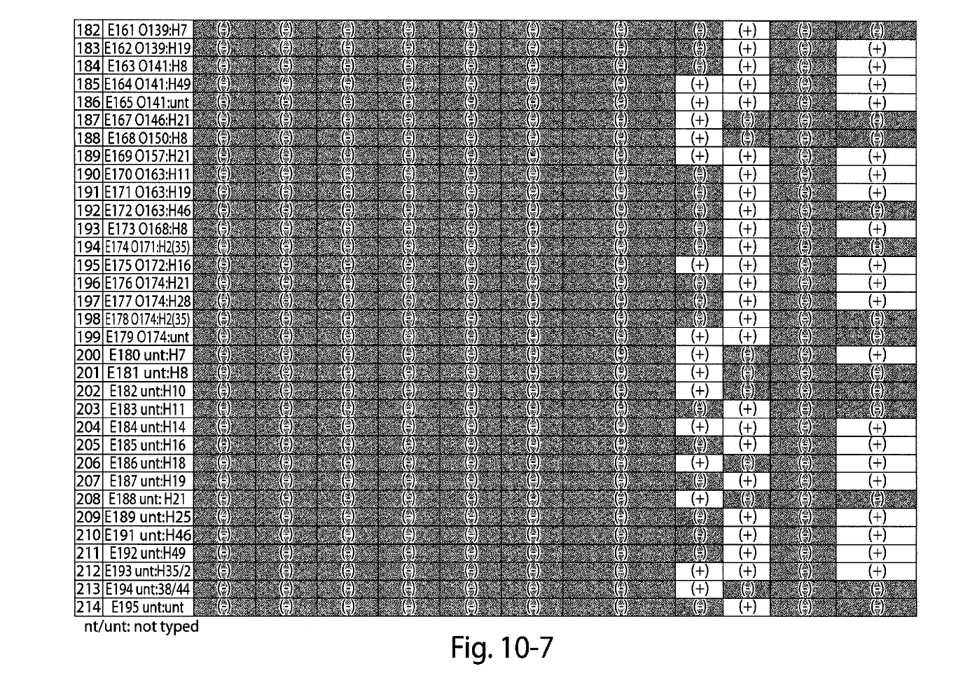

Accordingly, 214 E. coli strains shown in FIG. 10 were cultured according to standard methods. DNA was extracted from an overnight culture and purified using a PureLink Genomic DNA Kit (Invitrogen) according to kit instructions.

For sequencing, amplified DNA products were generated using a Clontech PCR kit consisting of the following master mix/reaction:

| Master Mix | Per Rxn |
|---|---|
| 10X Titanium Taq PCR Buffer | 6 uL |
| DNA template (100 ng/uL) | 3 uL |
| Primer Mix (10 uM each) | 2 uL |
| 50X dNTP mix (10 mM each of dATP, sCTP, dGTP, dTTP) | 1 uL |
| 50X Titanium Taq DNA Polymerase | 1 uL |
| Rnase-free H2O | 37 uL |
| Total Volume (per sample) | 50 uL |

Amplification conditions were as follows: 1 min at 95° C., 30 cycles of 30 seconds at 95° C. denature/90 seconds at 68° C. extension, followed by 90 seconds at 68° C. Amplified DNA was sequenced using oligos Z5866 F-1/Z5866 R-2 to detect target region Z5886 (O157:H7) and oligos ecf2-1 F/ecf2-1 R and ecf2-2 F/ecf 2-2 R) to detect target regions ecf2-1 and ecf2-2 (STEC). Sequences of these primers are shown below in Table 1.

TABLE 1

| | | |
|---|---|---|
| Z5866 | 5′-TTA ATT TTG ATG CCA | (SEQ ID |
| F-1 | GCC AGG TTG G-3′ | NO: 19) |
| Z5866 | 5′-GCT AGA TTC TGA CGT | (SEQ ID |
| R-2 | TAT TGC TGG TC-3′ | NO: 20) |
| ecf2- | 5′-AGG CAA GTA AAA CGG | (SEQ ID |
| 1F | AAT GTC CCT GC-3′ | NO: 21) |
| ecf2- | 5′-TAT GTT GAA TGC AAG | (SEQ ID |
| 1R | GCA TCT TCC CG-3′ | NO: 22) |
| ecf2- | 5′-GCT CTT TCG CAT TTA | (SEQ ID |
| 2F | ATC CAG TGG GA-3′ | NO: 23) |
| ecf2- | 5′-TAC AGC GGA ACG AAT | (SEQ ID |
| 2R | GGA ATA CGG GA-3′ | NO: 24) |

Real Time PCR analysis was performed as follows. A real time master mix using the following ratio of components was prepared: 10 ul Power ABI SYBR Green Mixture/7.8 ul RNase-free H₂O/0.2 ul Fwd/Rev primer. Primers are shown in Table 2. In a 96-well PCR plate, 2 ul of DNA template was added to 18 ul of real time master mix, sealed, and run on a Stratagene real time instrument using the following cycler conditions: denaturing for 10 minutes at 95° C., 40 cycles of 15 seconds at 95° C. denature/1 minute at 60° C. extension.

Replicates of each sample were run on Agilent Stratagene quantitative PCR machines for each respective primer pair and the data was subsequently compiled and analyzed using MxPro 3005P software.

TABLE 2

| | | |
|---|---|---|
| Z5886 (O157: | 5′-ATC TCC AAG GCG | (SEQ ID |
| H7)-F | GCA ACG AAA-3′ | NO: 25) |
| Z5886 (O157: | 5′-CAG AAG GTT ATG AAG | (SEQ ID |
| H7)-R | TTG AGT TCA TTC CAG-3′ | NO: 26) |
| ecf (STEC)- | 5′-CCC TTA TGA AGA GCC | (SEQ ID |
| F | AGT ACT GAA G-3′ | NO: 1) |
| ecf (STEC)- | 5′-ATT ACG CAT AGG GCG | (SEQ ID |
| R | TAT CAG CAC-3′ | NO: 2) |
| stx1F | 5′-ATA AAT CGC CAT TCG | (SEQ ID |
| | TTG ACT AC-3′ | NO: 27) |
| stx1R | 5′-AGA ACG CCC ACT GAG | (SEQ ID |
| | ATC ATC-3′ | NO: 28) |
| stx2F | 5′-GGC ACT GTC TGA AAC | (SEQ ID |
| | TGC TCC-3′ | NO: 29) |
| stx2R | 5′-TCG CCA GTT ATC TGA | (SEQ ID |
| | CAT TCT G-3′ | NO: 30) |
| eaeSTEC- | 5′-CAT TGA TCA GGA TTT | (SEQ ID |
| F | TTC TGG TGA TA-3′ | NO: 31) |
| eaeSTEC- | 5′-CTC ATG CGG AAA TAG | (SEQ ID |
| R | CCG TTA-3′ | NO: 32) |
| rfbO157- | 5′-CTGGACTCAACGTGGATTT | (SEQ ID |
| v | CATCA-3′ | NO: 33) |
| rfbO157- | 5′-ACCTAACGCTAACAAAGCT | (SEQ ID |
| R | AAATGAAG-3′ | NO: 34) |
| hlyASTEC- | 5′-GTG TCA GTA GGG AAG | (SEQ ID |
| F | CGA ACA-3′ | NO: 35) |
| hlyASTEC- | 5′-ATC ATG TTT TCC GCC | (SEQ ID |
| R | AAT G-3′ | NO: 36) |
| wzx1-F | 5′-TGC GTG TGG CAA AAA | (SEQ ID |
| | TTT AAA GAT-3′ | NO: 37) |
| wzx1-R | 5′-GTT GCC AAT CAA TCA | (SEQ ID |
| | TGC CAG AAG-3′ | NO: 38) |
| wzx2-F | 5′-AGT TAG GCA CTC TGG | (SEQ ID |
| | CAA CAT GGA-3′ | NO: 39) |
| wzx2-R | 5′-ATG AGC ATC TGC ATA | (SEQ ID |
| | AGC AGC CCA-3′ | NO: 40) |
| Z0344-F | 5′-CCT CTC AAT TGT CAG | (SEQ ID |
| | GGA AAT TAG CGT-3′ | NO: 41) |
| Z0344-R | 5′-TGT TAA TGG TTG AAC | (SEQ ID |
| | CGA CGG CAG-3′ | NO: 42) |
| Z0372-F | 5′-GGA CGA CGA ATA AAT | (SEQ ID |
| | GTC ACT CCA CC-3′ | NO: 43) |
| Z0372-R | 5′-CAG CCT GGA TAC CGC | (SEQ ID |
| | TAC TCA AAT-3′ | NO: 44) |
| wzy-F | 5′-CAG TTA CTA CGT ATG | (SEQ ID |
| | GAG CAG AAC TGT-3′ | NO: 45) |
| wzy-R | 5′-CGA TGC ATT CCC AGC | (SEQ ID |
| | CAC TAA GTA-3′ | NO: 46) |
| SIL-F | 5′-ATG AAT GCG CTG ACA | (SEQ ID |
| | ACC GAT GTG-3′ | NO: 47) |
| SIL-R | 5′-AAC TGT TGG TGC GTT | (SEQ ID |
| | TGG GTT ACG-3′ | NO: 48) |

Multiple E. coli STECs including O157:H7 and virulent non-O157 STECs such as O26, O45, O103, O111, O121, and O145 as well as non-virulent E. coli strains were tested. The data obtained from these PCR assays is summarized in FIG. 10. In particular, FIG. 10 shows PCR screening results testing 214 E. coli strains for specificity of O157:H7 (Z5886, rfb_{O157}, wzx_{O157}, Z0344, Z0372) and STEC (ecf) specific targets. In particular, these results show the specificity of the O157:H7 (Z5886, rfb_{O157}, wzx_{O157}, Z0344, Z0372) and STEC (ecf) specific targets, in addition to the genetic virulence profiles (stx1, stx2, eae, and hlyA). These data also demonstrate the specificity of O157 targets rfb_{O157}, wzx_{O157}, and Z0372 in combination with the ecf target region. The results also show that STEC (ecf) specific target detects only E. coli strains which have a combination of 3 virulence factors: stx1 or stx2 or stx1/stx2 in combination with eae_{STEC} and hlyA (ehx), and therefore is specific for highly virulent STEC/EHEC strains including the big six non-O157 serogroups—O26, O45, O103, O111, O121, and O145.

Further, we obtained 104 non-O157:H7 STEC isolates from the USDA (Bosilevac and Koohmaraie, Appl. Environ. Microbiol. 77(6):2103-2112, 2011). These isolates were tested with an O157:H7 specific target (Z5886), two O157 specific targets (rfb_{O157} and wzx_{O157}), and an ecf fragment. As shown in Table 3 none of the non-O157:H7 STEC isolates were detected by the O157:H7 or O157 specific targets. Of the 104 non-O157:H7 STEC isolates, 6 were the so-called big six non-O157:H7 STEC isolates. These were detected by a PCR assay specific for the ecf fragment. One out of 104 non-O157 STEC isolates was detected by the ecf PCR assay but does not belong to the group of big six non-O157 STEC. This sample is a highly virulent EHEC/STEC isolate which contains three virulent markers, stx, eae and hlyA, and therefore is correctly detected by the ecf assay herein.

TABLE 3
Specificity of O157 and STEC target regions tested by real time PCR (104
non-O157 STEC samples were tested).

| | | O157 | | | | | | STEC | |
|---|---|---|---|---|---|---|---|---|---|
| | | Z5886 | | rfb | | wax | | ecf | |
| | n | pos | neg | pos | neg | pos | neg | pos | neg |
| O157:H7 | 0 | 0 | 0 | 0 | 0 | 0 | 0 | 0 | 0 |
| O157:NM | 0 | 0 | 0 | 0 | 0 | 0 | 0 | 0 | 0 |
| Top 6 non-O157 | 6 | 0 | 6 | 0 | 6 | 6 | 6 | 6 | 0 |
| STEC | | | | | | | | | |
| Non-top 6 non- | 1 | 0 | 1 | 0 | 1 | 0 | 1 | 1 | 0 |
| O157 STEC/ | | | | | | | | | |
| EHEC | | | | | | | | | |
| Others | 97 | 0 | 97 | 0 | 97 | 0 | 97 | 0 | 97 |
| Total strains | 104 | | | | | | | | |
| tested | | | | | | | | | |

### Example 2

### wzx_{O157} and ecf1

A combination of two unique target genes (wzx_{O157} and ecf1) has been identified as allowing for the specific detection of virulent E. coli O157:H7 strains. Here we described the sensitivity and specificity of an E. coli O157:H7 detection assay using a collection of 480 E. coli O157:H7 and non-pathogenic E. coli isolates of different serotypes.

Methods: The E. coli O157:H7 detection assay combines two unique target genes, the chromosomal wzx_{O157} gene and the ecf1 gene which is located in a conserved ecf operon on a large virulence plasmid. The large virulence plasmid is found in highly virulent EHEC strains. The ecf operon encodes 4 proteins involved in cell wall synthesis which enhances colonization of E. coli in cattle. The sensitivity of the assay was determined by using serial 10-fold dilutions of five different E. coli O157:H7 strains. The sensitivity or limit of detection (LOD) was defined using a 95% confidence interval. We also determined the specificity of the assay by testing 480 inclusive and exclusive E. coli isolates, consisting of 117 E. coli O157:H7 and O157:NM strains, 7 non-virulent E. coli O157:NM strains, and 356 pathogenic and non-pathogenic non-O157 E. coli isolates including 130 of the FSIS regulated big six STEC strains. All isolates were tested at a concentration of 1e8 CFU/ml. Serotypes and presence of virulence genes such as shiga toxins 1 and 2 (stx₁ and stx₂), intimin (eae) and enterohemolysin (ehxA) for all E. coli isolates included in this study were tested by PCR.

Results: The LOD of the E. coli O157:H7 detection assay was determined to be 1e3 CFU/mL. All 117 O157H7/NM strains containing stx genes and the eae gene were successfully detected by the assay. Seven O157:NM strains which lacked shiga toxin genes were not detected. Of the 356 non-O157:H7 E. coli isolates included in this study, none were detected by the E. coli O157:H7 detection assay.

Significance: The results of these studies show that the use of the ecf1 gene in conjunction with the wzx_{O157}, gene accurately detects stx/eae containing pathogenic O157:H7/NM strains. These data demonstrate that the O157:H7 detection assay has 100% specificity and an analytical LOD of 1e3 CFU/mL.

### Example 3

### Use of the Ecf1 Gene to Detect Shiga Toxin-Producing Escherichia coli Strains in Beef Samples

Below we describe methods using primers to the ecf1 gene of the ecf operon for detecting enterohemorrhagic Escherichia coli strains (EHECs). E. coli O157:H7 and six serovars (O26, O103, O121, O111, O145, O45) are frequently implicated in severe clinical illness worldwide. Standard testing methods using stx, eae and O-serogroup-specific gene sequences for detecting the top six serogroups bear the disadvantage that these genes may reside, independently, in different non-pathogenic organisms leading to false positive results. The ecf operon has previously been identified in the large enterohemolysin-containing plasmid of eae-positive STEC. Here we disclose the utility of the ecf operon as a single marker to detect eae-positive STEC from pure culture and primary meat enrichments. Analysis of 501 E. coli isolates demonstrated a strong correlation between the presence of the ecf1 gene and the combined presence of stx, eae and ehxA genes. Two large studies were carried out to determine the utility of an ecf1-detection assay to detect non-O157 STEC strains in enriched meat samples in comparison to the FSIS-based method that detects stx and eae genes. In ground beef samples (n=1065), top six non-O157 STEC were detected in 4.0% of samples by an ecf1-detection assay and in 5.0% of samples by the stx/eae-based method. In contrast, in beef samples composed largely of trim (n=1097) top six non-O157 STEC were detected at 1.1% by both methods. Estimation of false positive rates among the top six non-O157 STEC revealed a lower rate using the ecf1 detection method (0.5%) compared to the eae/stx screening method (1.1%). Additionally, the ecf1-detection assay detected STEC strains associated with severe illness not included in the FSIS regulatory definition of adulterant STEC.

### Materials and Methods

Bacterial strains. E. coli strains included in this study (n=501) were acquired from Silliker Laboratories, United States Department of Agriculture (USDA) Agricultural Research Service, E. coli Reference Center Pennsylvania State University, STEC Center Michigan State University, and American Type Culture Collection (ATCC). Serotypes and presence of ecf1 and virulence genes stx₁, stx₂, eae, and ehxA of all E. coli isolates are provided in detail in Tables 4a and 4b. Approximately 30% of the E. coli isolates included in this study were from food sources. Bacterial isolates were stored frozen at −70° C. in brain heart infusion (BHI) media (Becton, Dickinson and Company, Franklin Lakes, N.J.) containing 30% glycerol and were subcultured on MacConkey agar plates (Hardy Diagnostics, Santa Maria, Calif.) prior to testing.

TABLE 4a
Presence of ecf1 and other virulence markers in E. coli O157: H7 and
E. coli O157: NM isolates

| | | Virulence Markers | | | | | |
|---|---|---|---|---|---|---|---|
| # | Isolates | ecf1 | stx₁ | stx₂ | eae | ehxA | Source |
| 1 | E. coli O157:H7 | + | + | + | + | + | apple cider |
| 2 | E. coli O157:H7 | + | + | + | + | + | sausage |
| 3 | E. coli O157:H7 | + | + | + | + | + | chesse curds |
| 4 | E. coli O157:H7 | + | + | + | + | + | USDA |
| | | | | | | | Culture |
| 5 | E. coli O157:H7 | + | + | + | + | + | salami |
| | | | | | | | outbreak |
| 6 | E. coli O157:H7 | + | + | + | + | + | pig feces |
| 7 | E. coli O157:H7 | + | + | + | + | + | clinical |
| 8 | E. coli O157:H7 | + | + | − | + | + | clinical |
| 9 | E. coli O157:H7 | + | + | + | + | + | ground beef |
| 10 | E. coli O157:H7 | + | + | + | + | + | ground beef |
| 11 | E. coli O157:H7 | + | + | + | + | + | ground beef |
| 12 | E. coli O157:H7 | + | + | + | + | + | ground beef |
| 13 | E. coli O157:H7 | + | + | + | + | + | ground beef |
| 14 | E. coli O157:H7 | + | + | − | + | + | ground beef |
| 15 | E. coli O157:H7 | + | + | + | + | + | ground beef |
| 16 | E. coli O157:H7 | + | + | + | + | + | ground beef |
| 17 | E. coli O157:H7 | + | + | + | + | + | ground beef |
| 18 | E. coli O157:H7 | + | + | + | + | + | ground beef |
| 19 | E. coli O157:H7 | + | + | + | + | + | ground beef |
| 20 | E. coli O157:H7 | + | + | + | + | + | food isolate |
| 21 | E. coli O157:H7 | + | + | + | + | + | ground beef |
| 22 | E. coli O157:H7 | + | + | + | + | + | pork |
| 23 | E. coli O157:H7 | + | + | + | + | + | food |
| | | | | | | | (hamburger) |
| 24 | E. coli O157:H7 | + | + | + | + | + | human |
| 25 | E. coli O157:H7 | + | + | + | + | + | human |
| 26 | E. coli O157:H7 | + | + | − | + | + | human |
| 27 | E. coli O157:H7 | + | − | + | + | + | human |
| 28 | E. coli O157:H7 | + | + | + | + | + | human |
| 29 | E. coli O157:H7 | + | + | + | + | + | human |
| 30 | E. coli O157:H7 | + | + | + | + | + | human |
| 31 | E. coli O157:H7 | + | + | + | + | + | human |
| 32 | E. coli O157:H7 | + | − | + | + | + | human |
| 33 | E. coli O157:H7 | + | + | + | + | + | cow (calf) |
| 34 | E. coli O157:H7 | + | − | + | + | + | human |
| 35 | E. coli O157:H7 | + | + | + | + | + | buffalo |
| 36 | E. coli O157:H7 | + | + | − | + | + | human |
| 37 | E. coli O157:H7 | + | + | + | + | + | unknown |
| 38 | E. coli O157:H7 | + | + | + | + | + | unknown |
| 39 | E. coli O157:H7 | + | + | + | + | + | unknown |
| 40 | E. coli O157:H7 | + | + | + | + | + | unknown |
| 41 | E. coli O157:H7 | + | + | + | + | + | unknown |
| 42 | E. coli O157:H7 | + | + | + | + | + | unknown |
| 43 | E. coli O157:H7 | + | + | + | + | + | unknown |
| 44 | E. coli O157:H7 | + | − | + | + | + | unknown |
| 45 | E. coli O157:H7 | + | + | − | + | + | unknown |
| 46 | E. coli O157:H7 | + | − | + | + | + | unknown |
| 47 | E. coli O157:H7 | + | − | + | + | + | unknown |
| 48 | E. coli O157:H7 | + | − | + | + | + | ground beef |
| 49 | E. coli O157:H7 | + | − | + | + | + | food isolate |
| 50 | E. coli O157:H7 | + | − | + | + | + | food isolate |
| 51 | E. coli O157:H7 | + | − | + | + | + | human |
| 52 | E. coli O157:H7 | + | − | + | + | + | cow (calf) |
| 53 | E. coli O157:H7 | + | + | + | + | + | unknown |
| 54 | E. coli O157:H7 | + | − | + | + | + | cattle |
| 55 | E. coli O157:H7 | + | − | + | + | + | cattle |
| 56 | E. coli O157:H7 | + | − | + | + | + | cattle |
| 57 | E. coli O157:H7 | + | − | + | + | + | cattle |
| 58 | E. coli O157:H7 | + | − | + | + | + | cattle |
| 59 | E. coli O157:H7 | + | + | + | + | + | cattle |
| 60 | E. coli O157:H7 | + | + | − | + | + | cattle |
| 61 | E. coli O157:H7 | + | + | + | + | + | cattle |
| 62 | E. coli O157:H7 | + | + | + | + | + | cattle |
| 63 | E. coli O157:H7 | + | − | + | + | + | cattle |
| 64 | E. coli O157:H7 | + | − | + | + | + | cattle |
| 65 | E. coli O157:H7 | + | + | − | + | + | cattle |
| 66 | E. coli O157:H7 | + | + | + | + | + | cattle |
| 67 | E. coli O157:H7 | + | + | − | + | + | cattle |
| 68 | E. coli O157:H7 | + | + | + | + | + | cattle |
| 69 | E. coli O157:H7 | + | + | − | + | + | cattle |
| 70 | E. coli O157:H7 | + | + | + | + | + | cattle |
| 71 | E. coli O157:H7 | + | + | + | + | + | cattle |
| 72 | E. coli O157:H7 | + | + | + | + | + | cattle |
| 73 | E. coli O157:H7 | + | + | − | + | + | cattle |
| 74 | E. coli O157:H7 | + | + | − | + | + | cattle |
| 75 | E. coli O157:H7 | + | + | + | + | + | cattle |
| 76 | E. coli O157:H7 | + | + | − | + | + | cattle |
| 77 | E. coli O157:H7 | + | − | + | + | + | cattle |
| 78 | E. coli O157:H7 | + | − | + | + | + | cattle |
| 79 | E. coli O157:H7 | + | + | − | + | + | cattle |
| 80 | E. coli O157:H7 | + | − | + | + | + | cattle |
| 81 | E. coli O157:H7 | + | + | + | + | + | cattle |
| 82 | E. coli O157:H7 | + | + | + | + | + | cattle |
| 83 | E. coli O157:H7 | + | − | + | + | + | cattle |
| 84 | E. coli O157:H7 | + | + | + | + | + | cattle |
| 85 | E. coli O157:H7 | + | − | + | + | + | cattle |
| 86 | E. coli O157:H7 | + | + | + | + | + | cattle |
| 87 | E. coli O157:H7 | + | + | + | + | + | cattle |
| 88 | E. coli O157:H7 | + | − | + | + | + | cattle |
| 89 | E. coli O157:H7 | + | − | + | + | + | cattle |
| 90 | E. coli O157:H7 | + | − | + | + | + | cattle |
| 91 | E. coli O157:H7 | + | − | + | + | + | cattle |
| 92 | E. coli O157:H7 | + | − | + | + | + | cattle |
| 93 | E. coli O157:H7 | + | + | + | + | + | cattle |
| 94 | E. coli O157:H7 | + | − | + | + | + | cattle |
| 95 | E. coli O157:H7 | + | − | + | + | + | cattle |
| 96 | E. coli O157:H7 | + | − | + | + | + | cattle |
| 97 | E. coli O157:H7 | + | − | + | + | + | cattle |
| 98 | E. coli O157:H7 | + | − | + | + | + | cattle |
| 99 | E. coli O157:H7 | + | − | + | + | + | unknown |
| 100 | E. coli O157:H7 | + | − | + | + | + | meat |
| | Rough | | | | | | |
| 101 | E. coli O157- | + | − | + | + | + | human |
| | NM | | | | | | |
| 102 | E. coli O157- | + | + | + | + | + | unknown |
| | NM | | | | | | |
| 103 | E. coli O157- | + | − | + | + | + | unknown |
| | NM | | | | | | |
| 104 | E. coli O157- | + | − | + | + | + | unknown |
| | NM | | | | | | |
| 105 | E. coli O157- | + | + | − | + | + | unknown |
| | NM | | | | | | |
| 106 | E. coli O157- | + | + | + | + | + | unknown |
| | NM | | | | | | |
| 107 | E. coli O157- | + | − | + | + | + | human |
| | NM | | | | | | (child) |
| 108 | E. coli O157- | + | + | + | + | + | human |
| | NM | | | | | | |
| 109 | E. coli O157- | + | + | + | + | + | human |
| | NM | | | | | | |
| 110 | E. coli O157- | + | − | + | + | + | food |
| | NM | | | | | | |
| 111 | E. coli O157- | + | + | + | + | + | cow |
| | NM | | | | | | |
| 112 | E. coli O157- | + | + | + | + | + | cow |
| | NM | | | | | | |
| 113 | E. coli O157- | + | + | − | + | + | unknown |
| | NM | | | | | | |
| 114 | E. coli O157- | + | + | + | + | + | unknown |
| | NM | | | | | | |
| 115 | E. coli O157- | + | + | + | + | + | cow |
| | NM | | | | | | |
| 116 | E. coli O157- | + | − | + | + | + | HC |
| | NM | | | | | | |
| 117 | E. coli O157- | + | − | + | + | + | HC |
| | NM | | | | | | |
| 118 | E. coli O157- | − | − | − | − | − | unknown |
| | NM | | | | | | |
| 119 | E. coli O157- | − | − | − | − | − | cattle |
| | NM | | | | | | |
| 120 | E. coli O157- | − | − | − | − | − | cattle |
| | NM | | | | | | |
| 121 | E. coli O157- | − | − | − | − | − | cattle |
| | NM | | | | | | |
| 122 | E. coli O157- | − | − | − | − | − | cattle |
| | NM | | | | | | |
| 123 | E. coli O157- | − | − | − | − | − | pig |
| | NM | | | | | | |
| 124 | E. coli O157- | − | − | − | − | − | human |
| | NM | | | | | | |

TABLE 4b
Presence of ecf1 and other virulence markers in non O157:H7 E. coli
isolates

| | | Virulence Markers | | | | | |
|---|---|---|---|---|---|---|---|
| # | Isolates | ecf1 | stx₁ | stx₂ | eae | ehxA | Source |
| 125 | E. coli O26 | + | + | − | + | + | human |
| 126 | E. coli O26:N | + | + | − | + | + | human |
| | | | | | | | (child, 6 y) |
| 127 | E. coli O26-H11 | + | + | − | + | + | human |
| 128 | E. coli O26:H11 | + | + | + | + | + | human |
| | | | | | | | (F, 2 y) |
| 129 | E. coli O26:H11 | + | + | − | + | + | ground Beef |
| 130 | E. coli O26:H11 | + | + | − | + | + | beef trim |
| 131 | E. coli O26:H8 | + | + | − | + | + | beef trim |
| 132 | E. coli O26:H11 | + | + | + | + | + | unknown |
| 133 | E. coli O26:H30 | + | + | − | + | + | Feces |
| 134 | E. coli O26:NM | + | + | − | + | + | conure, feces |
| 135 | E. coli O26:H11 | + | + | − | + | + | cow |
| 136 | E. coli O26:N | + | + | − | + | + | cow |
| 137 | E. coli O26:H11 | + | + | − | + | + | unknown |
| 138 | E. coli O26:H11 | + | + | − | + | + | unknown |
| 139 | E. coli O26:NM | + | + | + | + | + | unknown |
| 140 | E. coli O26:H11 | + | + | − | + | + | unknown |
| 141 | E. coli O26:H11 | + | + | + | + | + | unknown |
| 142 | E. coli O26:H11 | + | + | − | + | + | unknown |
| 143 | E. coli O26 | + | + | − | + | + | unknown |
| 144 | E. coli O26 | + | + | − | + | + | unknown |
| 145 | E. coli O26 | + | + | − | + | + | unknown |
| 146 | E. coli O26 | + | + | − | + | + | unknown |
| 147 | E. coli O26 | + | + | − | + | + | unknown |
| 148 | E. coli O26 | + | + | − | + | + | unknown |
| 149 | E. coli O26 | + | + | − | + | + | unknown |
| 150 | E. coli O26 | + | + | − | + | + | unknown |
| 151 | E. coli O26 | + | + | − | + | + | unknown |
| 152 | E. coli O26A | + | + | + | + | + | unknown |
| 153 | E. coli O26B | + | + | + | + | + | unknown |
| 154 | E. coli O45:NM | + | + | − | + | + | human |
| | | | | | | | (F, 77 y) |
| 155 | E. coli O45:H2 | + | + | − | + | + | human |
| | | | | | | | (M, 12 y) |
| 156 | E. coli O45:H2 | + | + | − | + | + | human |
| | | | | | | | (M, 45 y) |
| 157 | E. coli O45:H2 | + | + | − | + | + | human |
| | | | | | | | (F, 38 y) |
| 158 | E. coli O45:H2 | + | + | − | + | + | unknown |
| 159 | E. coli O45:H2 | + | + | − | + | + | unknown |
| 160 | E. coli O45:H2 | + | + | − | + | + | unknown |
| 161 | E. coli O45:H2 | + | + | − | + | + | unknown |
| 162 | E. coli O103:H2 | + | + | − | + | + | human |
| 163 | E. coli O103:H2(35) | + | + | − | + | + | ground beef |
| 164 | E. coli O103:H2(35) | + | + | − | + | + | ground beef |
| 165 | E. coli O103:H2(35) | + | + | − | + | + | ground beef |
| 166 | E. coli O103:H2(35) | + | + | − | + | + | ground beef |
| 167 | E. coli O103:H6 | + | + | − | + | + | human |
| 168 | E. coli O103:H25 | + | + | − | + | + | human |
| | | | | | | | (F, 3 y) |
| 169 | E. coli O103:N | + | + | − | + | + | human |
| 170 | E. coli O103:H2 | + | + | − | + | + | Horse |
| 171 | E. coli O103:H6 | + | + | − | + | + | human |
| 172 | E. coli O103:NM | + | + | − | + | + | human |
| 173 | E. coli O103:NM | + | + | − | + | + | human |
| 174 | E. coli O103:H11 | + | + | + | + | + | unknown |
| 175 | E. coli O103:H2 | + | + | − | + | + | unknown |
| 176 | E. coli O103:H2 | + | + | − | + | + | unknown |
| 177 | E. coli O103:H25 | + | + | − | + | + | unknown |
| 178 | E. coli O103:H8 | + | + | − | + | + | unknown |
| 179 | E. coli O103:H2 | + | + | − | + | + | unknown |
| 180 | E. coli O103:H2 | + | + | − | + | + | unknown |
| 181 | E. coli O103:H11 | + | + | − | + | + | unknown |
| 182 | E. coli O103:H2 | + | + | − | + | + | unknown |
| 183 | E. coli O103:H2 | + | + | − | + | + | unknown |
| 184 | E. coli O103:H2 | + | + | − | + | + | unknown |
| 185 | E. coli O103:H2 | + | + | − | + | + | unknown |
| 186 | E. coli O103 | + | + | − | + | + | unknown |
| 187 | E. coli O103 | + | + | + | + | + | unknown |
| 188 | E. coli O103:H12 | + | + | − | + | + | cow |
| 189 | E. coli O111:NM | + | + | + | + | + | human |
| | | | | | | | (M, 67 y) |
| 190 | E. coli O111− | + | + | + | + | + | unknown |
| 191 | E. coli O111:H8 | + | + | − | + | + | unknown |
| 192 | E. coli O111:H8 | + | + | + | + | + | human |
| | | | | | | | (F, 18 y) |
| 193 | E. coli O111:H11 | + | + | − | + | + | human |
| 194 | E. coli O111:H8 | + | + | + | + | + | unknown |
| 195 | E. coli O111:H28 | + | + | − | + | + | human |
| 196 | E. coli O111:NM | + | + | − | + | + | pig |
| 197 | E. coli O111:H11 | + | + | − | + | + | cow |
| 198 | E. coli O111:NM | + | + | + | + | + | unknown |
| 199 | E. coli O111:H11 | + | + | − | + | + | cow |
| 200 | E. coli O111:NM | + | + | + | + | + | cow |
| 201 | E. coli O111:NM | + | + | + | + | + | unknown |
| 202 | E. coli O111:NM | + | + | + | + | + | cow |
| 203 | E. coli O111:NM | + | + | − | + | + | unknown |
| 204 | E. coli O111:H8 | + | + | − | + | + | unknown |
| 205 | E. coli O111:[H8] | + | + | + | + | + | unknown |
| 206 | E. coli O111:H8 | + | + | − | + | + | unknown |
| 207 | E. coli O111 | + | + | + | + | + | unknown |
| 208 | E. coli O111:NM | + | + | + | + | + | unknown |
| 209 | E. coli O111:NM | + | + | + | + | + | unknown |
| 210 | E. coli O111:H8 | + | + | − | + | + | unknown |
| 211 | E. coli O111 | + | + | − | + | + | unknown |
| 212 | E. coli O111 | + | + | + | + | + | unknown |
| 213 | E. coli O111 | + | + | + | + | + | unknown |
| 214 | E. coli O111 | + | + | + | + | + | unknown |
| 215 | E. coli O121:[H19] | + | − | + | + | + | human |
| | | | | | | | (F, 51 y) |
| 216 | E. coli O121:H19 | + | − | + | + | + | human |
| 217 | E. coli O121 | + | − | + | + | + | human |
| 218 | E. coli O121:H19 | + | − | + | + | + | unknown |
| 219 | E. coli O121:H19 | + | − | + | + | + | unknown |
| 220 | E. coli O121:NM | + | − | + | + | + | unknown |
| 221 | E. coli O121:H19 | + | − | + | + | + | unknown |
| 222 | E. coli O121:H19 | + | − | + | + | + | unknown |
| 223 | E. coli O121:H19 | + | − | + | + | + | unknown |
| 224 | E. coli O121:H19 | + | − | + | + | + | unknown |
| 225 | E. coli O145:[28] | + | − | + | + | + | human |
| 226 | E. coli O145:H28 | + | + | − | + | + | ground beef |
| 227 | E. coli O145:NM | + | + | − | + | + | human |
| 228 | E. coli O145:NT | + | − | + | + | + | human |
| 229 | E. coli O145:+ | + | − | + | + | + | unknown |
| 230 | E. coli O145 | + | + | − | + | + | ground beef |
| 231 | E. coli O145:+ | + | − | + | + | + | food |
| 232 | E. coli O145:NM | + | + | + | + | + | cow |
| 233 | E. coli O145:NM | + | + | − | + | + | unknown |
| 234 | E. coli O145:H28 | + | − | + | + | + | unknown |
| 235 | E. coli O145:NM | + | − | + | + | + | unknown |
| 236 | E. coli O145:NM | + | + | − | + | + | unknown |
| 237 | E. coli O145:NM | + | + | − | + | + | unknown |
| 238 | E. coli O145:NM | + | + | − | + | + | unknown |
| 239 | E. coli O145:H2 | + | + | − | + | + | unknown |
| 240 | E. coli O145:H2 | + | + | − | + | + | unknown |
| 241 | E. coli O145A | + | + | + | + | + | unknown |
| 242 | E. coli O145B | + | + | + | + | + | unknown |
| 243 | E. coli O145C | + | + | + | + | + | unknown |
| 244 | E. coli O157:H43 | − | − | − | − | − | unknown |
| 245 | E. coli O157:H1 | − | − | − | + | − | unknown |
| 246 | E. coli O157:H2 | − | − | − | + | − | rabbit |
| 247 | E. coli O157:H4 | − | − | − | − | − | chicken |
| 248 | E. coli O157:H5 | − | − | − | − | − | food |
| 249 | E. coli O157:H8 | − | − | − | + | − | human |
| 250 | E. coli O157:H12 | − | − | − | − | − | water |
| 251 | E. coli O157:H15 | − | − | − | − | − | unknown |
| 252 | E. coli O157:H16 | − | − | − | + | − | dog |
| 253 | E. coli O157:H19 | − | − | − | − | − | pig |
| 254 | E. coli O157:H29 | − | − | − | − | − | food |
| 255 | E. coli O157:H29 | − | − | − | − | − | unknown |
| 256 | E. coli O157:H32 | − | − | − | − | − | cow |
| 257 | E. coli O157:H39 | − | − | − | + | − | human |
| 258 | E. coli O157:H42 | − | − | − | − | − | unknown |
| 259 | E. coli O157:H43 | − | − | − | − | − | unknown |
| 260 | E. coli O157:H45 | − | − | − | − | − | unknown |
| 261 | E. coli O55:H6 | − | − | − | + | − | unknown |
| 262 | E. coli O55:NM | − | − | − | + | − | unknown |
| 263 | E. coli O55:H7 | − | − | + | + | − | unknown |
| 264 | E. coli O55:H7 | − | − | − | + | − | unknown |
| 265 | E. coli O55:H7 | − | − | − | + | − | unknown |
| 266 | E. coli O55:H7 | − | − | − | + | − | unknown |
| 267 | E. coli O2:NM | − | − | − | − | − | unknown |
| 268 | E. coli O4:H40 | − | − | − | − | − | unknown |
| 269 | E. coli | − | − | − | − | − | unknown |
| | O7:K1(L):NM | | | | | | |
| 270 | E. coli O25:HN | − | − | − | − | − | unknown |
| 271 | E. coli O75:NM | − | − | − | − | − | unknown |
| 272 | E. coli O79:NM | − | − | − | − | − | unknown |
| 273 | E. coli O85:HN | − | − | − | − | − | unknown |
| 274 | E. coli O91:H7 | − | − | − | + | − | unknown |
| 275 | E. coli O91:H21 | − | − | + | − | + | unknown |
| 276 | E. coli O104:H21 | − | − | − | − | − | unknown |
| 277 | E. coli O104:H21 | − | − | − | − | − | unknown |
| 278 | E. coli O111:H2 | − | − | + | − | − | unknown |
| 279 | E. coli O111:H2 | − | − | − | + | − | unknown |
| 280 | E. coli O113:H21 | − | − | − | − | − | unknown |
| 281 | E. coli O113:H21 | − | − | + | − | − | unknown |
| 282 | E. coli O121:HN | − | − | − | − | − | unknown |
| 283 | E. coli O121:H19 | − | − | + | − | + | unknown |
| 284 | E. coli ECOR-51 | − | − | − | − | − | unknown |
| 285 | E. coli ON:HN | − | − | − | + | − | unknown |
| 286 | E. coli unt:H18 | − | − | − | − | − | horse |
| 287 | E. coli unt:H27 | − | − | − | − | − | cow |
| 288 | E. coli O1:H11 | − | − | + | − | + | ground beef |
| 289 | E. coli O1:H19 | − | − | + | − | + | ground beef |
| 290 | E. coli O5:H7 | − | + | + | − | + | ground beef |
| 291 | E. coli O5:H14 | − | + | + | − | + | ground beef |
| 292 | E. coli O8:H8 | − | − | + | − | + | ground beef |
| 293 | E. coli O8:H16 | − | + | − | − | − | ground beef |
| 294 | E. coli O8:H19 | − | + | + | − | + | ground beef |
| 295 | E. coli O8:H25 | − | + | − | − | − | ground beef |
| 296 | E. coli O8:H49 | − | − | + | − | + | ground beef |
| 297 | E. coli O15:H27 | − | + | + | − | − | ground beef |
| 298 | E. coli O17:45 | − | + | + | − | + | ground beef |
| 299 | E. coli O20:H7 | − | + | + | − | + | ground beef |
| 300 | E. coli O20:H19 | − | + | + | − | + | ground beef |
| 301 | E. coli O20:unt | − | + | − | − | + | ground beef |
| 302 | E. coli O22:H8 | − | + | + | − | + | ground beef |
| 303 | E. coli O22:H11 | − | + | + | − | + | ground beef |
| 304 | E. coli O22:H19 | − | − | + | − | + | ground beef |
| 305 | E. coli O22:H19 | − | + | − | − | − | ground beef |
| 306 | E. coli O22:H49 | − | − | + | − | + | ground beef |
| 307 | E. coli O22:unt | − | + | + | − | + | ground beef |
| 308 | E. coli unt:H21 | − | − | + | − | − | ground beef |
| 309 | E. coli O41:H11 | − | − | + | − | + | ground beef |
| 310 | E. coli O41:H25 | − | − | + | − | + | ground beef |
| 311 | E. coli O41:H35 | − | − | + | − | + | ground beef |
| 312 | E. coli O41:H2(35) | − | − | + | − | + | ground beef |
| 313 | E. coli unt:H7 | − | − | + | − | − | ground beef |
| 314 | E. coli O48:H7 | − | + | + | − | + | ground beef |
| 315 | E. coli O74:H8 | − | + | − | − | − | ground beef |
| 316 | E. coli O74:H28 | − | + | − | − | − | ground beef |
| 317 | E. coli O74:H42 | − | + | − | − | + | ground beef |
| 318 | E. coli O82:H8 | − | + | + | − | + | ground beef |
| 319 | E. coli O86:H8 | − | − | + | − | + | ground beef |
| 320 | E. coli O88:H25 | − | − | + | − | + | ground beef |
| 321 | E. coli O88:unt | − | − | + | − | + | ground beef |
| 322 | E. coli O91:H10 | − | − | + | − | − | ground beef |
| 323 | E. coli O91:H14 | − | + | + | − | + | ground beef |
| 324 | E. coli O91:H21 | − | − | + | − | + | ground beef |
| 325 | E. coli O101:H19 | − | + | − | − | − | ground beef |
| 326 | E. coli O91:H21 | − | + | + | − | + | ground beef |
| 327 | E. coli unt:H2(35) | − | − | + | − | + | ground beef |
| 328 | E. coli O104:H7 | − | + | − | − | + | ground beef |
| 329 | E. coli O105:H7 | − | − | + | − | + | ground beef |
| 330 | E. coli O105:H18 | − | + | + | − | + | ground beef |
| 331 | E. coli O109:H5 | − | + | − | − | − | ground beef |
| 332 | E. coli O109:H48 | − | + | − | − | + | ground beef |
| 333 | E. coli O112:H8 | − | − | + | − | + | ground beef |
| 334 | E. coli O112:H19 | − | − | + | − | + | ground beef |
| 335 | E. coli O112:H45 | − | + | − | − | − | ground beef |
| 336 | E. coli O112:H2(35) | − | − | + | − | − | ground beef |
| 337 | E. coli O112:unt | − | − | + | − | + | ground beef |
| 338 | E. coli O113:H21 | − | − | + | − | + | ground beef |
| 339 | E. coli O116:H21 | − | + | + | − | + | ground beef |
| 340 | E. coli O116:unt | − | − | + | − | + | ground beef |
| 341 | E. coli unt:H7 | − | − | + | − | − | ground beef |
| 342 | E. coli unt:H35/2 | − | − | + | − | + | ground beef |
| 343 | E. coli O121:H7 | − | + | − | − | − | ground beef |
| 344 | E. coli O121:H7 | − | + | − | − | − | ground beef |
| 345 | E. coli O121:H7 | − | + | − | − | − | ground beef |
| 346 | E. coli O121:H7 | − | + | − | − | − | ground beef |
| 347 | E. coli O121:H7 | − | + | − | − | − | ground beef |
| 348 | E. coli unt:H8 | − | − | + | − | + | ground beef |
| 349 | E. coli unt:H16 | − | + | + | − | + | ground beef |
| 350 | E. coli unt:H19 | − | − | + | − | − | ground beef |
| 351 | E. coli O139:H7 | − | − | + | − | − | ground beef |
| 352 | E. coli O139:H19 | − | − | + | − | + | ground beef |
| 353 | E. coli O141:H8 | − | − | + | − | + | ground beef |
| 354 | E. coli O141:H49 | − | + | + | − | + | ground beef |
| 355 | E. coli O141:unt | − | + | + | − | + | ground beef |
| 356 | E. coli O146:H21 | − | + | − | − | − | ground beef |
| 357 | E. coli O150:H8 | − | + | − | − | − | ground beef |
| 358 | E. coli unt:H21 | − | + | + | − | + | ground beef |
| 359 | E. coli O163:H11 | − | − | + | − | + | ground beef |
| 360 | E. coli O163:H19 | − | − | + | − | + | ground beef |
| 361 | E. coli O163:H46 | − | − | + | − | − | ground beef |
| 362 | E. coli O168:H8 | − | − | + | − | + | ground beef |
| 363 | E. coli O171:H2(35) | − | − | + | − | − | ground beef |
| 364 | E. coli O172:H16 | − | + | + | − | + | ground beef |
| 365 | E. coli O174:H21 | − | − | + | − | + | ground beef |
| 366 | E. coli O174:H28 | − | − | + | − | + | ground beef |
| 367 | E. coli O174:H2(35) | − | − | + | − | − | ground beef |
| 368 | E. coli O174:unt | − | + | + | − | − | ground beef |
| 369 | E. coli unt:H7 | − | + | − | − | + | ground beef |
| 370 | E. coli unt:H8 | − | + | − | − | − | ground beef |
| 371 | E. coli unt:H10 | − | + | − | − | − | ground beef |
| 372 | E. coli unt:H11 | − | − | + | − | − | ground beef |
| 373 | E. coli unt:H14 | − | + | + | − | + | ground beef |
| 374 | E. coli unt:H16 | − | − | + | − | + | ground beef |
| 375 | E. coli unt:H18 | − | + | − | − | + | ground beef |
| 376 | E. coli unt:H19 | − | − | + | − | + | ground beef |
| 377 | E. coli unt:H21 | − | + | − | − | − | ground beef |
| 378 | E. coli unt:H25 | − | − | + | − | + | ground beef |
| 379 | E. coli unt:H46 | − | − | + | − | + | ground beef |
| 380 | E. coli unt:H49 | − | − | + | − | + | ground beef |
| 381 | E. coli unt:H35/2 | − | + | + | − | + | ground beef |
| 382 | E. coli unt:H38/44 | − | + | − | − | − | ground beef |
| 383 | E. coli unt:unt | − | − | + | − | − | ground beef |
| 384 | E. coli O171:H | − | + | + | − | + | beef trim |
| 385 | E. coli O88:H38 | − | + | + | − | + | beef trim |
| 386 | E. coli unt:H | − | − | + | − | − | beef trim |
| 387 | E. coli O113:H36 | − | − | + | − | + | beef trim |
| 388 | E. coli O33:H11 | − | − | + | − | + | beef trim |
| 389 | E. coli O116:H21 | − | + | + | − | + | beef trim |
| 390 | E. coli O73:H | − | + | − | − | − | beef trim |
| 391 | E. coli O73:H35 | − | + | − | − | − | beef trim |
| 392 | E. coli O64:H9 | − | − | + | − | − | beef trim |
| 393 | E. coli OX25:H11 | − | − | + | − | + | beef trim |
| 394 | E. coli unt:H34 | − | + | − | − | − | beef trim |
| 395 | E. coli O113:H21 | − | − | + | − | + | beef trim |
| 396 | E. coli O20:H19 | − | − | + | − | − | beef trim |
| 397 | E. coli O142:H34 | − | + | − | − | − | beef trim |
| 398 | E. coli O55, 83:H15 | − | − | + | − | − | beef trim |
| 399 | E. coli O113:H51 | − | − | + | − | + | beef trim |
| 400 | E. coli O39:H14 | − | − | + | − | − | beef trim |
| 401 | E. coli unt:H19 | − | − | + | − | − | beef trim |
| 402 | E. coli O132:H38 | − | − | + | − | − | beef trim |
| 403 | E. coli O8:H3 | − | + | + | − | + | beef trim |
| 404 | E. coli O168:+ | − | − | + | − | + | beef trim |
| 405 | E. coli O163:H19 | − | − | + | − | − | beef trim |
| 406 | E. coli O172:H10 | − | − | + | − | − | beef trim |
| 407 | E. coli O130:H11 | − | + | + | − | + | beef trim |
| 408 | E. coli unt:H11 | − | − | + | − | − | beef trim |
| 409 | E. coli O174:H28 | − | − | + | − | + | beef trim |
| 410 | E. coli O82:H8 | − | + | + | − | + | beef trim |
| 411 | E. coli O83:H8 | − | − | + | − | − | beef trim |
| 412 | E. coli O6:H34 | − | − | + | − | − | beef trim |
| 413 | E. coli unt:H52 | − | − | + | − | + | beef trim |
| 414 | E. coli O113:H4 | − | + | + | − | − | beef trim |
| 415 | E. coli unt:H18 | − | + | − | − | + | beef trim |
| 416 | E. coli O26:H2 | − | − | − | − | − | human |
| 417 | E. coli O45:H16 | − | − | − | − | − | unknown |
| 418 | E. coli O45:NM | − | − | − | + | − | pig |
| 419 | E. coli O45:H9 | − | − | − | − | − | chicken |
| 420 | E. coli O45:H30 | − | − | − | − | − | pig |
| 421 | E. coli O45:H10 | − | − | − | − | − | chicken |
| 422 | E. coli O45:H18 | − | − | − | − | − | human |
| 423 | E. coli O45:H25 | − | − | − | − | − | human |
| 424 | E. coli O45:H4 | − | − | − | − | − | turkey |
| 425 | E. coli O103:H21 | − | − | − | − | − | turkey |
| 426 | E. coli O103:H11 | − | − | − | − | − | mouse |
| 427 | E. coli O103:N | − | − | − | − | − | cow |
| 428 | E. coli O121:H4 | − | + | − | − | − | cow |
| 429 | E. coli O121:H44 | − | + | − | − | − | cow |
| 430 | E. coli O121:H10 | − | − | + | − | − | food |
| 431 | E. coli O121:H7 | − | − | − | − | − | ferret |
| 432 | E. coli O121:NM | − | − | − | − | − | pig |
| 433 | E. coli O121:H10 | − | − | − | − | − | pig |
| 434 | E. coli O121:H10 | − | − | − | − | − | pig |
| 435 | E. coli O121:H7 | − | + | − | − | − | cow |
| 436 | E. coli O121:H6 | − | − | − | − | − | avian |
| 437 | E. coli O145:NM | − | − | − | + | − | rabbit |
| 438 | E. coli O145:H7 | − | − | − | + | − | rabbit |
| 439 | E. coli O145:H34 | − | − | − | + | − | dog |
| 440 | E. coli O145:H2 | − | − | − | + | − | rabbit |
| 441 | E. coli O113:H21 | − | − | + | − | + | unknown |
| 442 | E. coli O55:H7 | − | + | − | + | − | unknown |
| 443 | E. coli O91:H21 | − | − | + | − | + | unknown |
| 444 | E. coli O174:H8 | − | + | + | − | − | unknown |
| 445 | E. coli O55:H7 | − | + | − | + | − | unknown |
| 446 | E. coli O128ac:[H2] | − | + | − | + | − | unknown |
| 447 | E. coli O113:H4 | − | + | + | − | + | unknown |
| 448 | E. coli O41:H26 | − | + | − | − | − | unknown |
| 449 | E. coli O138 | − | − | − | − | − | unknown |
| 450 | E. coli O91:H21 | − | − | + | − | + | unknown |
| 451 | E. coli O2 | − | − | + | − | + | unknown |
| 452 | E. coli O121 | − | − | − | + | − | unknown |
| 453 | E. coli O121 | − | − | − | + | − | unknown |
| 454 | E. coli O111:NM | − | − | + | − | + | unknown |
| 455 | E. coli O111 | − | − | − | + | − | unknown |
| 456 | E. coli O121:H19 | − | − | + | − | + | unknown |
| 457 | E. coli O113:H21 | − | − | + | − | + | HUS |
| 458 | E. coli O104:H4 | − | − | + | − | − | HUS |
| 459 | E. coli O91:H21 | − | − | + | − | + | HUS |
| 460 | E. coli O36:H14 | − | − | + | − | + | sprouts |
| 461 | E. coli O113:H21 | − | − | + | − | + | spinach |
| 462 | E. coli O168:H− | − | − | + | − | + | lettuce |
| 463 | E. coli O113:H21 | − | − | + | − | + | spinach |
| 464 | E. coli O113:H21 | + | + | − | + | + | Canada |
| 465 | E. coli O125:NM | + | + | − | + | + | USA (N.C.) |
| 466 | E. coli O165:H− | + | − | + | + | + | beef trim |
| 467 | E. coli O165:H25 | + | − | + | + | + | unknown |
| 468 | E. coli O5:NM | + | + | − | + | + | unknown |
| 469 | E. coli O177:[H25] | + | − | + | + | + | unknown |
| 470 | E. coli unt:H16 | + | + | − | + | + | human |
| 471 | E. coli unt:H25 | + | + | − | + | + | unknown |
| 472 | E. coli non-O157:H7 | + | − | + | + | + | cattle |
| | STEC | | | | | | |
| 473 | E. coli unt:H2 | + | + | − | + | + | beef trim |
| 474 | E. coli O157:H12 | + | + | + | + | + | pig |
| 475 | E. coli O157:H19 | + | − | + | + | + | primate |
| 476 | E. coli O26 | + | − | − | + | + | dog |
| 477 | E. coli O26 | + | − | − | + | + | cow |
| 478 | E. coli O26 | − | − | − | + | − | cow |
| 479 | E. coli O26 | − | − | − | + | − | cow |
| 480 | E. coli O26 | + | − | − | + | + | cow |
| 481 | E. coli O103 | + | − | − | + | + | deer |
| 482 | E. coli O103 | + | − | − | + | + | deer |
| 483 | E. coli O103 | + | − | − | + | + | deer |
| 484 | E. coli O103 | + | − | − | + | + | cow |
| 485 | E. coli O145 | + | − | − | + | + | cow |
| 486 | E. coli O145 | + | − | − | + | + | cow |
| 487 | E. coli O145 | + | − | − | + | + | cow |
| 488 | E. coli O145 | + | − | − | + | + | cow |
| 489 | E. coli O145 | + | − | − | + | + | cow |
| 490 | E. coli O103:H12 | − | + | − | + | + | cow |
| 491 | E. coli O26 | − | + | − | + | − | cow |
| 492 | E. coli O26:H11 | − | + | − | + | − | unknown |
| 493 | E. coli O103:H11 | − | + | − | + | − | human |
| 494 | E. coli O103:H2 | − | + | − | + | − | cow |
| 495 | E. coli O111:H8 | − | + | − | + | − | food |
| 496 | E. coli O111:NM | − | − | + | + | − | unknown |
| 497 | E. coli O111:H8 | − | + | − | + | − | unknown |
| 498 | E. coli O121:H19 | − | − | + | + | − | unknown |
| 499 | E. coli O145:H2 | − | − | + | + | − | rabbit |
| 500 | E. coli O145:NM | − | − | + | + | − | human |
| 501 | E. coli O145:NM | − | + | − | + | − | unknown |

unt: untyped

Ground Beef and Beef Trim Samples:

A total of 2162 pre-enriched beef samples were examined. One set of enriched ground beef samples (n=1065) were received from a commercial ground beef producer and came from 78-85% lean finished ground beef (Study I). All beef samples received had been pre-screened for E. coli O157:H7 and only negative samples were supplied. The supplier prepared randomized samples of different sizes (25 g, 50 g, 75 g or 100 g) which were diluted 1:10 (225 mL, 450 mL, 675 mL, or 900 mL) in tryptic soy broth (Becton, Dickinson and Company, Franklin Lakes, N.J.) and then enriched for 14-20 hrs at 42° C. After enrichment, 10 mL of broth was collected from each sample and shipped over night on ice to Roka Bioscience where samples were frozen at −70° C. until further processing. A second set (n=1097) of enriched beef trim (n=881) and ground beef (n=216) samples were obtained from an independent certified testing laboratory (Study II). The testing laboratory diluted 375 g of ground beef or trim 1:10 in RapidChek® E. coli O157 Enrichment Media (Strategic Diagnostics Inc., Newark Del.) and then enriched for 12-18 hours at 42° C. After enrichment, 3.6 mL of broth was collected from each sample and placed into collection tubes containing 6 mL of Roka transfer media, a proprietary solution that efficiently lyses bacterial cells, releases bacterial nucleic acid and stabilizes the nucleic acid for up to 5 days at room temperature. The samples were then shipped over night on ice to Roka Bioscience where samples were frozen at −70° C. until further processing.

Preparation of Template DNA from Bacterial Cultures:

Template DNA from pure bacterial cultures was prepared using PureLink™ Genomic DNA Kits (Invitrogen, Carlsbad Calif.). A single colony from a MacConkey agar plate was diluted in 5 mL BHI broth and grown overnight at 35° C. One mL was then pelleted by centrifugation and used in the PureLink™ Genomic DNA extraction kit according to the manufacturer's specified protocol. Aliquots of 2 to 5 μl of the final DNA preparation were then directly transferred to the PCR reactions or stored at −20° C. until further analysis.

Preparation of Template DNA from Enriched Beef Samples:

Template DNA from the 1065 enriched ground beef samples received from the commercial ground beef producer (Study I) was prepared according to the PrepMan® Ultra Sample Preparation Reagent Protocol (Applied Biosystems, Foster City, Calif.). One mL of enrichment broth was centrifuged for 3 min. The supernatant was discarded and 100 μL PrepMan® Ultra Sample Preparation Reagent was added. After heating at 100° C. for 10 minutes the extract was centrifuged and 50 μl was diluted into 450 μl of nuclease-free water. Aliquots of 2 to 5 μl of this DNA preparation were then directly transferred to the PCR reactions or stored at −20° C. until further analysis. Nucleic acid was extracted from the second set of 1097 enriched beef samples (Study II) using the KingFisher® 96 magnetic particle processor (Thermo Fisher Scientific, Waltham, Mass.) followed by PCR analysis. An aliquot of 400 μl from each sample was combined with 125 μl of Roka target capture reagent containing magnetic beads that bind nucleic acids. The solution was heated to 95° C. for 10 minutes using an EchoTherm™ SC20 Orbital Mixing Chilling/Heating Dry Bath (Torrey Pines Scientific, Carlsbad Calif.). The samples were placed on the KingFisher® 96 magnetic particle processor, magnetic beads were collected and transferred into 200 μl of Roka wash buffer containing detergent. The samples were mixed, collected and washed a second time. The final elution of the nucleic acid bound to magnetic beads was captured in a volume of 50 μl consisting of 25 μl TagMan® Environmental Master Mix 2.0, 21 μl RNase-free H₂O, and 4.0 μl probe (375 nM), forward and reverse primers (2.5 uM each).

PCR Assays to Determine Presence of Ecf, Virulence Genes and O-Serogroups:

The presence of the ecf1 gene, virulence factors stx₁, stx₂, eae, ehxA and presence of O-serogroups O26, O45, O103, O111, O121, O145 was determined in 501 E. coli isolates and 2162 enriched beef samples using real time PCR. The presence of ecf3 and ecf4 was also determined in 253 out of the 501 E. coli isolates. The presence of virulence factors was determined using stx₁, stx₂ specific oligonucleotides (Paton et al., 1998. Journal of clinical microbiology 36:598-602) and eae, ehxA specific oligonucleotides (Bugarel et al. 2010. Appl Environ Microbiol 76:203-211) as previously described. The presence of O-serogroup-specific gene sequences for O26, O45, O103, O111, O121, or O145 were determined as described in USDA/FSIS MLG5B.03 Appendix 1.01. All other target specific oligos are listed in Table 5. For real time PCR amplification reactions, either the Power SYBR® Green PCR Master Mix or TagMan® Environmental Master Mix 2.0 was used (Applied Biosystems, Foster City, Calif.). All PCR amplification reactions using the Power SYBR® Green PCR Master Mix were performed in a final volume of 20 μl consisting of 10 μl Power SYBR® Green PCR Master Mix, 7.8 μl RNase-free H₂O, and 0.2 μl forward and reverse primers (1.5 μM each). PCR amplification reactions using the TagMan® Environmental Master Mix 2.0 were performed in a final volume of 25 μl consisting of 12.5 μl TagMan® Environmental Master Mix 2.0, 8.5 μl RNase-free H₂O, and 2.0 μl probe (150 nM), forward and reverse primers (1.0 μM each), with the exception of beef samples from the second study that used a final volume of 50 μl as described in the previous section. Purified DNA (2-5 μl) isolated from E. coli isolates and Study I enriched beef samples was used as the source of template DNA and added to the PCR reaction mixtures. Template DNA for the beef samples from Study II utilized nucleic acid extracted using the KingFisher® 96 magnetic particle processor as described above. Samples were amplified with an initial denaturation step at 95° C. for 10 min. Then the following thermocycling conditions for the individual amplification reactions were 40 cycles (SYBR® Green) or 45 cycles (TagMan®) of denaturation at 95° C. for 15 sec, annealing and extension at 60° C. for 1 min (SYBR® Green) or 59° C. for 1 min (TaqMan®), followed by 15 sec at 95° C., 15 sec at 60° C., and 15 sec at 95° C. All PCR reactions were performed on the Agilent Mx3005P quantitative real time PCR instrument (Santa Clara, Calif.). A sample was considered positive if the Cycles to Threshold (Ct) values were ≦30 using the SYBR® Green method, or ≦35 using the TagMan® method. Determination of the cutoff value was based on the limit of detection of a known positive control. Melting curve analysis was performed to confirm the specificity of amplicons in SYBR® Green PCR reactions using the default settings of the device.

TABLE 5
Primer and probe sequences for real-time PCR detection of genetic markers used in this study

| Target | | | | Gene | |
|---|---|---|---|---|---|
| Gene/ | | SEQ | Location | Bank | |
| Genetic | Forward primer, reverse primer | ID | within | Acces- | Refer- |
| Element | and probe sequence(5′-3′)^{a} | NO | sequence | sion | ence |
| bfpA | CCA GTC TGC GTC TGA TTC CA | 123 | 2756-2775 | FM180569 | 6 |
| | CGT TGC GCT CAT TAC TTC TGA A | 124 | 2816-2795 | | |
| Eae | CAT TGA TCA GGA TTT TTC TGG TGA TA | 125 | 4394375-4394350 | CP003109 | 2 |
| | CTC ATG CGG AAA TAG CCG TTA | 126 | 4394274-4394294 | | |
| | [FAM]ATA GTC TCG CCA GTA TTC GCC ACC AAT | 127 | 4394309-4394338 | | |
| | ACC[IBFQ] | | | | |
| ehxA | GTG TCA GTA GGG AAG CGA ACA | 128 | 30082-30062 | AP010959 | 3 |
| | ATC ATG TTT TCC GCC AAT G | 129 | 29957-29975 | | |
| | [FAM]TCT GTT GAA GAGCTC ATT GGC GGA[IBFQ] | 130 | 29989-29966 | | |
| ecf1 | TAT CAG CAC CAA AGA GCG GGA ACA | 131 | 18668-18691 | AP010959 | This |
| | CCC TTA TGA AGA GCC AGT ACT GAA | 132 | 18766-18742 | | Study |
| | [FAM]AAA GGC GTC GTT TCA GCC AGC CGG AA[IBFQ] | 133 | 18692-18717 | | |
| ecf3 | TTC CTT TGC CAT GGC GGA GAA TTG | 134 | 20423-20446 | AP010959 | This |
| | AGC GGC TCC TGT CTG ATT AAC GAT | 135 | 20519-20496 | | Study |
| ecf4 | ACG CTG GAA TGG TCT GGA GAT TGT | 136 | 22180-22203 | AP010959 | This |
| | ATC CAC CAC CGG ATT TCT CTG GTT | 137 | 22345-22322 | | Study |
| efa1 | TTT CGC TCA CAA CAA TCG AA | 138 | 22152-22171 | AP010954 | This |
| | TTG GCC AAA AGA AAG TGT AGC | 139 | 22324-22304 | | Study |
| espK | ATT GTA ACT GAT GTT ATT TCG TTT GG | 140 | 1673295-1673320 | AE005174 | 6 |
| | GRC ATC AAA AGC GAA ATC ACA CC | 141 | 1673419-1673397 | | |
| espP | ACC ATG AAT GCG TGC TGT AA | 142 | 18785-18809 | AP010963 | This |
| | CTG GAC GGA CTG GAT TTG TT | 143 | 19016-18997 | | study |
| nleB | CAT GTT GAA GGC TGG AAS TTT GT | 144 | 5107730-5107708 | AP010958 | 3 |
| | CCG CTA CAG GGC GAT ATG TT | 145 | 5107659-5107678 | | |
| katP | TTT CAG GAA CGG TGA GAT CC | 146 | 24451-24432 | AP010963 | This |
| | CCC TTT ACT CCG GGA AGA AC | 147 | 24274-24293 | | study |
| RepA | GGC CGC TTT TCA GTT ATG AG | 148 | 14958-14939 | AP010963 | This |
| | CGA CCG GAG CCA CTT TAG TT | 149 | 14851-14870 | | study |
| stcE | GAG AGC AGC ACT TTC GCT TT | 150 | 2519-2500 | AP010959 | This |
| | TGG ATA CCC GAA CAC TCA CA | 151 | 2300-2319 | | Study |
| stx₁ | TTT GTY ACT GTS ACA GCW GAA GCY TTA CG | 152 | 5388250-388279 | AP010958 | 4 |
| | CCC CAG TTC ARW GTR AGR TCM ACD TC | 153 | 5388445-388420 | | |
| | [FAM]CTG GAT GAT CTC AGT GGG CGT TCT TAT GTA | 154 | 5388313-5388343 | | |
| | A[IBFQ] | | | | |
| stx₂ | TTT GTY ACT GTS ACA GCW GAA GCY TTA CG | 155 | 2897519-2897490 | AP010958 | 4 |
| | CCC CAG TTC ARW GTR AGR TCM ACD TC | 156 | 2897414-2897440 | | |
| | [FAM]TCG TCA GGC ACT GTC TGA AAC TGC TCC[IBFQ] | 157 | 2897489-2897463 | | |
| stx₁ | ATA AAT CGC CAT TCG TTG ACT AC | 158 | 5388157-5388179 | AP010958 | 1 |
| | AGA ACG CCC ACT GAG ATC ATC | 159 | 5388336-5388316 | | |
| stx₂ | GGC ACT GTC TGA AAC TGC TCC | 160 | 2897483-2897463 | AP010958 | 1 |
| | TCG CCA GTT ATC TGA CAT TCT G | 161 | 2897229-2897250 | | |
| traG | ATC TGC CCA CTC ATG CTT TC | 162 | 37441-37422 | AP010959 | This |
| | GGC CAG CGA TTA CTT TAC CA | 163 | 37244-37263 | | study |
| traT | CGG AGA AGT CAC CAC CTG AT | 164 | 38813-38794 | AP010959 | This |
| | TTG ATG ATG GTT GCA CTG GT | 165 | 38568-38587 | | study |
| T2SS | CTG CTC CGT TGT TGG GTA AC | 166 | 8555-8536 | AP010958 | This |
| | GCA TCA GCG TGG TTT TAC CT | 167 | 8357-8376 | | study |
| WZX_{O103d} | TTG GAG CGT TAA CTG GAC CT | 168 | 2519094-2519075 | AP010958 | 4 |
| | ATA TTC GCT ATA TCT TCT TGC GGC | 169 | 2518904-2518927 | | |
| | [FAM]AGGCTTATCTGGCTGTTCTTACTACGGC[IBFQ] | 170 | 2518986-2518959 | | |
| WZX_{O111} | TGT TCC AGG TGG TAG GAT TCG | 171 | 2690613-2690593 | AP010960 | 4 |
| | TCA CGA TGT TGA TCA TCT GGG | 172 | 2690377-2690397 | | |
| | [FAM]TGAAGGCGAGGCAACACATTATATAGTGC[IBFQ] | 173 | 2690462-2690438 | | |
| WZX_{O121} | AGG CGC TGT TTG GTC TCT TAG A | 174 | 6839-6860 | AY208937 | 4 |
| | GAA CCG AAA TGA TGG GTG CT | 175 | 7027-7008 | | |
| | [FAM]CGCTATCATGGCGGGACAATGACAGTGC[IBFQ] | 176 | 6898-6925 | | |
| WZX_{O145} | AAA CTG GGA TTG GAC GTG G | 177 | 4968-4986 | AY863412 | 4 |
| | CCC AAA ACT TCT AGG CCC G | 178 | 5102-5084 | | |
| | [FAM]TGCTAATTGCAGCCCTTGCACTACGAGGC[IBFQ] | 179 | 5018-5046 | | |
| WZX_{O26} | GTA TCG CTG AAA TTA GAA GCG C | 180 | 2862267-2862246 | AP010953 | 4 |
| | AGT TGA AAC ACC CGT AAT GGC | 181 | 2862110-2862130 | | |
| | [FAM]TGGTTCGGTTGGATTGTCCATAAGAGGG[IBFQ] | 182 | 2862185-2862158 | | |
| WZX_{O45} | CGT TGT GCA TGG TGG CAT | 183 | 7472-7489 | AY771223 | 4 |
| | TGG CCA AAC CAA CTA TGA ACT G | 184 | 7542-7522 | | |
| | [FAM]ATTTTTTCGTCGAAGTGGGCTGTACA[IBFQ] | 185 | 7494-7517 | | |
| Z2098 | CTG AAA AGA GCC AGA ACG TGC | 186 | 1888173-1888193 | AE005174 | 5 |
| | TGC CTA AGA TCA TTA CCC GGA C | 187 | 1888308-1888287 | | |
| Z2099 | TAG CGG GAC AAT TGT CAC GG | 188 | 1889124-1889143 | AE005174 | 5 |
| | GTC TTT CGG AGA AAC ATT CTG CC | 189 | 1889190-1889168 | | |
| pO103 | CTG CGA CAC GGT ATC TGA AA | 190 | 14100-14119 | AP010959 | This |
| | ACC GAT AAA TGG GAC CAA CA | 191 | 14326-14307 | | Study |
| pO103 | CAC GAT GAC TGG CTG AAG AA | 192 | 15753-15772 | AP010959 | This |
| | CGG TAG TGC GGA CCT TTT TA | 193 | 15939-15920 | | Study |
| pO103 | ATG GCA GGT CTG CTA CAG GT | 194 | 17476-17495 | AP010959 | This |
| | TAG CGG AAT TTT CTG CAT CC | 195 | 17696-17627 | | Study |
| pO103 | ATC ATT GGC AAC ACT GGT GA | 196 | 29219-29238 | AP010959 | This |
| | AAA GAT GCC TCA GGA GCA GA | 197 | 29392-29373 | | Study |
| pO103 | TTC TTT CTC CCG ACA TCC AG | 198 | 32351-32370 | AP010959 | This |
| | TAT GGG CCT GTT CTC CTC TG | 199 | 32566-32547 | | Study |
| pO103 | TGT CAG CCA GAA CCA CTG AC | 200 | 34587-34606 | AP010959 | This |
| | GCC TTT TTC CTT GTC ATC CA | 201 | 34810-34791 | | Study |
| pO111 | TAT GGG CCT GTT CTC CTC TG | 202 | 44-63 | AP010963 | This |
| | TTC TTT CTC CCG ACA TCC AG | 203 | 259-240 | | Study |
| pO111 | CAA CCT GGA CAG GAG GTC AT | 204 | 6831-6850 | AP010963 | This |
| | GCA CCC CGG TTT TTA TTT CT | 205 | 7059-7040 | | Study |
| pO111 | GTG CAT GAT GTA TGG CAA GC | 206 | 27870-27889 | AP010963 | This |
| | GGA ACC CGG GAC TGT TTA AT | 207 | 28022-28003 | | Study |
| pO111 | AGT CAA CTA TCC GGG GGA AG | 208 | 34171-34190 | AP010963 | This |
| | CTG TGG GAT TTC CGT GAT TT | 209 | 34366-34347 | | Study |
| pO111 | AGA GTG AAG GGG AAC GAG GT | 210 | 64308-64327 | AP010963 | This |
| | TCC GGT AAC CAG AAC CTC AC | 211 | 64534-64515 | | Study |

^{a}FAM, fluorescein; IBFQ. Iowa Black FQ
1 Paton AW and Paton JC, J Clin Microbiol. 1998
2 Nielsen EM and Andersen MT, J Clin Microbiol. 2003
3 Bugarel M et al., Appl Environ Microbiol. 2010
4 FSIS MLG 5B.03
5 Delannoy S et al., J Clin Microbiol. 2013
6 Bugarel M et al BMC Microbiol 11:142. 2011

PCR Assays to Determine the Presence of Plasmid Sequences and Chromosomal Gene Sequences Associated with eae-Positive STEC.

Non-O157 E. coli isolates which were positive for the ecf1 and eae and ehxA genes but negative for stx₁ and stx₂ genes were tested by SYBR® Green real time PCR for the presence of the bfpA gene found only in typical enteropathogenic E. coli (EPEC). In addition, chromosomal gene markers associated with eae-positive STEC such as nleB, espK, Z2098, and Z2099 were tested. Non-O157 E. coli isolates positive for either stx₁ or stx₂ genes and eae genes but lacking ecf1 and ehxA genes were tested for the presence of additional plasmid genes, katP, efa1, stcE, T2SS, espP, tratT, and tratG. Non-O157 E. coli isolates that were negative for ecf1 and ehxA genes and positive for at least one plasmid gene were tested with additional PCR primers for plasmid sequences located on pO103 and pO111. All PCR primers used in this study are summarized in Table 5. E. coli isolates with the same O-serogroup were used as positive controls. All thermocycling conditions are described in the section above.

### Results

Screening of E. coli Isolates for Ecf Specificity

To investigate the specificity of ecf, we examined 501 E. coli isolates from various sources for the presence of ecf and other virulent genes including stx1, stx2, eae, and ehxA. We selected primers to the ecf-1 gene that are specific for E. coli and not other bacteria. As summarized in Table 6, 100 of 100 O157:H7 isolates were correctly identified including one rough strain not expressing the O antigen were positive for ecf1 gene. All of these isolates contained ecf, stx1 or stx2, eae and ehxA. Because O157:NM strains containing stx are also considered adulterants in beef by the FSIS, we examined 24 O157:NM strains. Only 17 O157:NM strains containing stx and eae and ehxA were also positive for the ecf1 gene while the remaining 7 E. coli O157:NM strains, which were negative for stx and eae and exhA genes were also negative for ecf1 gene (Table 4a).

TABLE 6
Presence of ecf1 and virulence markers in 501 E. coli isolates included in this study

| | | | | | Virulence Markers | | | |
|---|---|---|---|---|---|---|---|---|
| | Isolates | | | N | ecf1 | stx^{a} | eae | ehxA |
| ecf positive | E. coli O157:H7/Rough | | | 100 | + | + | + | + |
| E. coli | E. coli O157:NM | | | 17 | + | + | + | + |
| | Top six non-O157 STEC | O26 | | 29 | + | + | + | + |
| | (n = 119) | O45 | | 8 | + | + | + | + |
| | | O103 | | 27 | + | + | + | + |
| | | O111 | | 26 | + | + | + | + |
| | | O121 | | 10 | + | + | + | + |
| | | O145 | | 19 | + | + | + | + |
| | Other than top six non-O157 STEC | | | 12 | + | + | + | + |
| | stx (—) top six non-O157 STEC^{b} | | | 12 | + | − | + | + |
| ecf negative | Top six non-O157 STEC^{c} | | | 11 | − | + | + | − |
| | | | | {open oversize brace} | | | | |
| E. coli | (n = 12) | | | 1 | − | + | + | + |
| | E. coli O157:NM | | | 7 | − | − | − | − |
| | E. coli | | | 43 | − | − | − | − |
| | EPEC | | | 23 | − | − | + | − |
| | | | | 59 | − | + | − | − |
| | STEC (n = 156) | | {open oversize brace} | 93 | − | + | − | + |
| | | | | 4 | − | + | + | − |

^{a}Positive if stx₁ and/or stx₂ positive
^{b}Described further in Table 10
^{c}Described further in Tables 7 and 8

We then examined 131 Big 6 STEC strains for the presence of ecf1 (Table 6). Of these strains 119 contained ec1f, stx, eae and ehxA genes. The remaining 12 isolates were positive for stx, eae, and ehxA but were missing ecf1 (1 isolate) or were positive for stx and eae but were missing ecf1 and ehxA (11 isolates). These 12 strains were tested for additional plasmid markers including katP, efa1, stcE, traT, traG, T2SS, and espP. Nine of the twelve strains were missing all plasmid genes found in the positive controls isolates with the same O-serogroup (Table 7), while three strains demonstrated partial loss of plasmid sequences including sequences within the ehxA gene (Table 8). Although these 12 E. coli isolates were missing ecf1 and other plasmid markers characteristic of that strain, all 12 strains were positive for espK, nleB, Z2098, Z2099, chromosomal genes characteristic of eae-positive STEC strains (Table 7 and Table 8).

TABLE 7
Presence of plasmid and chromosomal gene markers in 9 E. coli isolates negative for ecf1 and ehxA and containing a top six O serogroup.

| Target | O26 | O26:H11 | O103:H11 | O111:H8 | O111:NM | O121:H19 | O145:H2 | O145:NM | O145:NM | STEC Controls | | | | |
|---|---|---|---|---|---|---|---|---|---|---|---|---|---|---|
| Gene | (#491)^{c} | (#492)^{c} | (#493)^{c} | (#495)^{c} | (#496)^{c} | (#498)^{c} | (#499)^{c} | (#500)^{c} | (#501)^{c} | O103 | O111 | O145 | O121 | O26 |
| ecf^{a} | − | − | − | − | − | − | − | − | − | + | + | + | + | + |
| ehxA^{a} | − | − | − | − | − | − | − | − | − | + | + | + | + | + |
| katP^{a} | − | − | − | − | − | − | − | − | − | − | + | + | − | + |
| traT^{a} | − | − | − | − | − | − | − | − | − | + | + | + | − | − |
| traG^{a} | − | − | − | − | − | − | − | − | − | + | + | + | − | − |
| efa1^{a} | − | − | − | − | − | − | − | − | − | − | − | + | + | + |
| T2SS^{a} | − | − | − | − | − | − | − | − | − | + | − | − | − | − |
| stcE^{a} | − | − | − | − | − | − | − | − | − | + | − | − | − | − |
| espK^{b} | + | + | + | + | + | + | + | + | + | + | + | + | + | + |
| Z2098^{b} | + | + | + | + | + | + | + | + | + | + | + | + | + | + |
| Z2099^{b} | + | + | + | + | + | + | + | + | + | + | + | + | + | + |
| nleB^{b} | + | + | + | + | + | + | + | + | + | + | + | + | + | + |
| stx^{bd} | + | + | + | + | + | + | + | + | + | + | + | + | + | + |
| eae^{b} | + | + | + | + | + | + | + | + | + | + | + | + | + | + |

^{a}Located on large enterohemolysin-containing plasmid
^{b}Located on chromosome
^{c}E. coli isolate number based on Table 4b
^{d}Positive if stx₁ and/or stx₂ positive

TABLE 8
Presence of plasmid sequences located on pO103 and pO111 and chromosomal gene markers in 3 E. coli
isolates negative for ecf1 and containing a top six O serogroup.

| | Plasmid | | | | | Plasmid | | |
|---|---|---|---|---|---|---|---|---|
| Target | Sequence | | | | Target | Sequence | | |
| Gene/ | location on | O103 | O103:H2 | O103:H12 | Gene/ | location on | O111 | O111:H8 |
| Locus Tag^{a} | pO103^{a} | Control | (#494)^{d} | (#490)^{d} | LocusTag^{b} | pO111^{b} | Control | (#497)^{d} |
| stcE | 2299-2523 | + | + | − | p3-01 | 44-259 | + | − |
| T2SS | 8357-8555 | + | + | − | ehxA | 2528-2653 | + | − |
| p14 | 14100-14326 | + | + | − | p3-04 | 6831-7059 | + | − |
| p17 | 15753-15939 | + | + | + | repA | 14851-14939 | + | + |
| p19 | 17476-17646 | + | − | − | espP | 18785-19016 | + | + |
| ecf1 | 18668-18766 | + | − | − | katP | 24274-24451 | + | − |
| ehxA | 29219-29392 | + | − | + | p3-31 | 27870-28022 | + | − |
| p31 | 29957-30082 | + | − | + | p3-40 | 34171-34366 | + | + |
| p32 | 32351-32566 | + | − | + | traG | 53598-53795 | + | + |
| p35 | 34587-34810 | + | + | + | traT | 54895-55140 | + | + |
| traG | 37244-37441 | + | + | + | p3-77 | 64308-64534 | + | − |
| traT | 38567-38813 | + | + | + | ecf1 | 72417-72515 | + | − |
| espK^{c} | | + | + | + | espK^{c} | | + | + |
| Z2098^{c} | | + | + | + | Z2098^{c} | | + | + |
| Z2099^{c} | | + | + | + | Z2099^{c} | | + | + |
| nleB^{c} | | + | + | + | nleB^{c} | | + | + |
| stx^{ce} | | + | + | + | stx^{c} | | + | + |
| eae^{c} | | + | + | + | eae^{c} | | + | + |

^{a}Located on pO103 NC_013354 Gen Bank # AP010959
^{b}Located on pO111 NC_013366 Gen Bank # AP010963
^{c}Located on chromosome
^{d}E. coli isolate number based on Table 4b
^{e}Positive if stx1 and/or stx2 positive

In addition to the six most frequent non-O157 STEC strains, the ecf1 target was also detected in other STEC serogroups containing stx and eae and ehxA genes. These included O5:NM, O113:H21, O125:NM, O165:H−, O165:H25, O157:H12, O157:H19, and O177(H25) serotypes, (Table 4b) several of which have been reported to be associated with HUS outbreaks (Sandhu et al. 2002. Can J Vet Res. 66:65-72, Uchida et al. 1995. The Journal of the Japanese Association for Infectious Diseases 69:678-683).

218 E. coli isolates negative for either stx or eae genes, including 43 E. coli strains with different serotypes, 23 EPEC strains and 152 STEC isolates, were negative for the ecf1 gene. Four STEC strains positive for stx and eae genes but negative for the six most frequent O serogroups were also negative for both ecf1 and ehxA genes (Table 6, Table 4b). All four isolates (three E. coli O55:H7 strains and one E. coli O128 strain) were also negative for the eae-positive STEC markers Z2098 and Z2099, except for one E. coli O55:H7 isolate which was positive for the Z2099 marker. In addition, 11 closely related bacterial organisms (Citrobacter braakii, Enterobacter cloacae, Hafnia alvei, Klebsiella oxytoca, Pantoea agglomerans, Proteus vulgaris, Providencia alcalifaciens, Salmonella Bongori, Serratia marcescens, Shigella flexneri, Yersinia enterocolitica) were tested and confirmed to be negative for the ecf1 specific sequence (data not shown).

To investigate whether other ecf genes within the ecf operon showed the same specificity as ecf1 we screened 253 E. coli isolates with primers to the ecf3 and ecf4 genes in addition to the ecf1 gene. Detection of ecf3 and ecf4 genes showed the same specificity as the ecf1 target (Table 9).

Finally, we examined 12 E. coli isolates of the six most frequent non-O157 strains missing stx genes. All twelve of these isolates were found to be ecf1 and eae and ehxA positive (Table 10). We tested these isolates for the presence of a typical EPEC marker (bfpA) as well as chromosomal gene markers characteristic of eae-positive STEC strains (espk, nleB, Z2098, Z2099)(see, for example, Bugarel et al. 2011. BMC Microbiology 11:142, Bugarel et al. 2010. Appl Environ Microbiol 76:203-211, Delannoy et al. 2013. Journal of clinical microbiology 51:1083-1088, and Bugarel et al. 2011. Appl Environ Microbiol 77:2275-2281). In each of the 12 E. coli isolates the typical EPEC marker was missing and 11 E. coli isolates were positive for all eae-positive STEC markers and one E. coli isolate was negative for the eae-positive STEC markers Z2098 and Z2099 but the eae-positive STEC markers espK and nleB were present (Table 10).

TABLE 9
Presence of ecf-1, ecf-3 and ecf-4 genes in 253 E. coli O157:H7 and non O157:H7 isolates

| | | Virulence Factors | | | | | | | |
|---|---|---|---|---|---|---|---|---|---|
| # | Isolates | ecf1 | ecf3 | ecf4 | stx1 | stx2 | eae | ehxA | Source |
| 1 | E. coli O157:H7 | + | + | + | + | + | + | + | apple cider |
| 2 | E. coli O157:H7 | + | + | + | + | + | + | + | sausage |
| 3 | E. coli O157:H7 | + | + | + | + | + | + | + | chesse curds |
| 4 | E. coli O157:H7 | + | + | + | + | + | + | + | USDA Culture |
| 5 | E. coli O157:H7 | + | + | + | + | + | + | + | salami outbreak |
| 6 | E. coli O157:H7 | + | + | + | + | + | + | + | pig feces |
| 7 | E. coli O157:H7 | + | + | + | + | + | + | + | clinical |
| 8 | E. coli O157:H7 | + | + | + | + | − | + | + | clinical |
| 9 | E. coli O157:H7 | + | + | + | + | + | + | + | ground beef |
| 10 | E. coli O157:H7 | + | + | + | + | + | + | + | ground beef |
| 11 | E. coli O157:H7 | + | + | + | + | + | + | + | ground beef |
| 12 | E. coli O157:H7 | + | + | + | + | + | + | + | ground beef |
| 13 | E. coli O157:H7 | + | + | + | + | + | + | + | ground beef |
| 14 | E. coli O157:H7 | + | + | + | + | − | + | + | ground beef |
| 15 | E. coli O157:H7 | + | + | + | + | + | + | + | ground beef |
| 16 | E. coli O157:H7 | + | + | + | + | + | + | + | ground beef |
| 17 | E. coli O157:H7 | + | + | + | + | + | + | + | ground beef |
| 18 | E. coli O157:H7 | + | + | + | + | + | + | + | ground beef |
| 19 | E. coli O157:H7 | + | + | + | + | + | + | + | ground beef |
| 20 | E. coli O157:H7 | + | + | + | + | + | + | + | food isolate |
| 21 | E. coli O157:H7 | + | + | + | + | + | + | + | ground beef |
| 22 | E. coli O157:H7 | + | + | + | + | + | + | + | pork |
| 23 | E. coli O157:H7 | + | + | + | + | + | + | + | food (hamburger) |
| 24 | E. coli O157:H7 | + | + | + | + | + | + | + | human |
| 25 | E. coli O157:H7 | + | + | + | + | + | + | + | human |
| 26 | E. coli O157:H7 | + | + | + | + | − | + | + | human |
| 27 | E. coli O157:H7 | + | + | + | − | + | + | + | human |
| 28 | E. coli O157:H7 | + | + | + | + | + | + | + | human |
| 29 | E. coli O157:H7 | + | + | + | + | + | + | + | human |
| 30 | E. coli O157:H7 | + | + | + | + | + | + | + | human |
| 31 | E. coli O157:H7 | + | + | + | + | + | + | + | human |
| 32 | E. coli O157:H7 | + | + | + | − | + | + | + | human |
| 33 | E. coli O157:H7 | + | + | + | + | + | + | + | cow (calf) |
| 34 | E. coli O157:H7 | + | + | + | − | + | + | + | human |
| 35 | E. coli O157:H7 | + | + | + | + | + | + | + | buffalo |
| 36 | E. coli O157:H7 | + | + | + | + | − | + | + | human |
| 37 | E. coli O157:H7 | + | + | + | + | + | + | + | unknown |
| 38 | E. coli O157:H7 | + | + | + | + | + | + | + | unknown |
| 39 | E. coli O157:H7 | + | + | + | + | + | + | + | unknown |
| 40 | E. coli O157:H7 | + | + | + | + | + | + | + | unknown |
| 41 | E. coli O157:H7 | + | + | + | + | + | + | + | unknown |
| 42 | E. coli O157:H7 | + | + | + | + | + | + | + | unknown |
| 43 | E. coli O157:H7 | + | + | + | + | + | + | + | unknown |
| 44 | E. coli O157:H7 | + | + | + | − | + | + | + | unknown |
| 45 | E. coli O157:H7 | + | + | + | + | − | + | + | unknown |
| 46 | E. coli O157:H7 | + | + | + | − | + | + | + | unknown |
| 47 | E. coli O157:H7 | + | + | + | − | + | + | + | unknown |
| 48 | E. coli O157:H7 | + | + | + | − | + | + | + | ground beef |
| 49 | E. coli O157:H7 | + | + | + | − | + | + | + | food isolate |
| 50 | E. coli O157:H7 | + | + | + | − | + | + | + | food isolate |
| 51 | E. coli O157:H7 | + | + | + | − | + | + | + | human |
| 52 | E. coli O157:H7 | + | + | + | − | + | + | + | cow (calf) |
| 53 | E. coli O157:H7 | + | + | + | + | + | + | + | unknown |
| 54 | E. coli O157:H7 | + | + | + | − | + | + | + | cattle |
| 55 | E. coli O157:H7 | + | + | + | − | + | + | + | cattle |
| 56 | E. coli O157:H7 | + | + | + | − | + | + | + | cattle |
| 57 | E. coli O157:H7 | + | + | + | − | + | + | + | cattle |
| 58 | E. coli O157:H7 | + | + | + | − | + | + | + | cattle |
| 59 | E. coli O157:H7 | + | + | + | + | + | + | + | cattle |
| 60 | E. coli O157:H7 | + | + | + | + | − | + | + | cattle |
| 61 | E. coli O157:H7 | + | + | + | + | + | + | + | cattle |
| 62 | E. coli O157:H7 | + | + | + | + | + | + | + | cattle |
| 63 | E. coli O157:H7 | + | + | + | − | + | + | + | cattle |
| 64 | E. coli O157:H7 | + | + | + | − | + | + | + | cattle |
| 65 | E. coli O157:H7 | + | + | + | + | − | + | + | cattle |
| 66 | E. coli O157:H7 | + | + | + | + | + | + | + | cattle |
| 67 | E. coli O157:H7 | + | + | + | + | − | + | + | cattle |
| 68 | E. coli O157:H7 | + | + | + | + | + | + | + | cattle |
| 69 | E. coli O157:H7 | + | + | + | + | − | + | + | cattle |
| 70 | E. coli O157:H7 | + | + | + | + | + | + | + | cattle |
| 71 | E. coli O157:H7 | + | + | + | + | + | + | + | cattle |
| 72 | E. coli O157:H7 | + | + | + | + | + | + | + | cattle |
| 73 | E. coli O157:H7 | + | + | + | + | − | + | + | cattle |
| 74 | E. coli O157:H7 | + | + | + | + | − | + | + | cattle |
| 75 | E. coli O157:H7 | + | + | + | + | + | + | + | cattle |
| 76 | E. coli O157:H7 | + | + | + | + | − | + | + | cattle |
| 77 | E. coli O157:H7 | + | + | + | − | + | + | + | cattle |
| 78 | E. coli O157:H7 | + | + | + | − | + | + | + | cattle |
| 79 | E. coli O157:H7 | + | + | + | + | − | + | + | cattle |
| 80 | E. coli O157:H7 | + | + | + | − | + | + | + | cattle |
| 81 | E. coli O157:H7 | + | + | + | + | + | + | + | cattle |
| 82 | E. coli O157:H7 | + | + | + | + | + | + | + | cattle |
| 83 | E. coli O157:H7 | + | + | + | − | + | + | + | cattle |
| 84 | E. coli O157:H7 | + | + | + | + | + | + | + | cattle |
| 85 | E. coli O157:H7 | + | + | + | − | + | + | + | cattle |
| 86 | E. coli O157:H7 | + | + | + | + | + | + | + | cattle |
| 87 | E. coli O157:H7 | + | + | + | + | + | + | + | cattle |
| 88 | E. coli O157:H7 | + | + | + | − | + | + | + | cattle |
| 89 | E. coli O157:H7 | + | + | + | − | + | + | + | cattle |
| 90 | E. coli O157:H7 | + | + | + | − | + | + | + | cattle |
| 91 | E. coli O157:H7 | + | + | + | − | + | + | + | cattle |
| 92 | E. coli O157:H7 | + | + | + | − | + | + | + | cattle |
| 93 | E. coli O157:H7 | + | + | + | + | + | + | + | cattle |
| 94 | E. coli O157:H7 | + | + | + | − | + | + | + | cattle |
| 95 | E. coli O157:H7 | + | + | + | − | + | + | + | cattle |
| 96 | E. coli O157:H7 | + | + | + | − | + | + | + | cattle |
| 97 | E. coli O157:H7 | + | + | + | − | + | + | + | cattle |
| 98 | E. coli O157:H7 | + | + | + | − | + | + | + | cattle |
| 99 | E. coli O157:H7 | + | + | + | − | + | + | + | unknown |
| 101 | E. coli O157-NM | + | + | + | − | + | + | + | human |
| 102 | E. coli O157-NM | + | + | + | + | + | + | + | unknown |
| 103 | E. coli O157-NM | + | + | + | − | + | + | + | unknown |
| 104 | E. coli O157-NM | + | + | + | − | + | + | + | unknown |
| 105 | E. coli O157-NM | + | + | + | + | − | + | + | unknown |
| 106 | E. coli O157-NM | + | + | + | + | + | + | + | unknown |
| 107 | E. coli O157-NM | + | + | + | − | + | + | + | human (child) |
| 108 | E. coli O157-NM | + | + | + | + | + | + | + | human |
| 109 | E. coli O157-NM | + | + | + | + | + | + | + | human |
| 110 | E. coli O157-NM | + | + | + | − | + | + | + | food |
| 111 | E. coli O157-NM | + | + | + | + | + | + | + | cow |
| 112 | E. coli O157-NM | + | + | + | + | + | + | + | cow |
| 113 | E. coli O157-NM | + | + | + | + | − | + | + | unknown |
| 114 | E. coli O157-NM | + | + | + | + | + | + | + | unknown |
| 115 | E. coli O157-NM | + | + | + | + | + | + | + | cow |
| 116 | E. coli O157-NM | + | + | + | − | + | + | + | HC |
| 117 | E. coli O157-NM | + | + | + | − | + | + | + | HC |
| 118 | E. coli O157-NM | − | − | − | − | − | − | − | unknown |
| 119 | E. coli O157-NM | − | − | − | − | − | − | − | cattle |
| 120 | E. coli O157-NM | − | − | − | − | − | − | − | cattle |
| 121 | E. coli O157-NM | − | − | − | − | − | − | − | cattle |
| 122 | E. coli O157-NM | − | − | − | − | − | − | − | cattle |
| 123 | E. coli O157-NM | − | − | − | − | − | − | − | pig |
| 124 | E. coli O157-NM | − | − | − | − | − | − | − | human |
| 125 | E. coli O26 | + | + | + | + | − | + | + | human |
| 126 | E. coli O26:N | + | + | + | + | − | + | + | human (child, 6y) |
| 127 | E. coli O26-H11 | + | + | + | + | − | + | + | human |
| 128 | E. coli O26:H11 | + | + | + | + | + | + | + | human (F, 2y) |
| 129 | E. coli O26:H11 | + | + | + | + | − | + | + | ground beef |
| 130 | E. coli O26:H11 | + | + | + | + | − | + | + | beef trim |
| 131 | E. coli O26:H8 | + | + | + | + | − | + | + | beef trim |
| 132 | E. coli O26:H11 | + | + | + | + | + | + | + | unknown |
| 133 | E. coli O26:H30 | + | + | + | + | − | + | + | feces |
| 134 | E. coli O26:NM | + | + | + | + | − | + | + | conure, feces |
| 135 | E. coli O26:H11 | + | + | + | + | − | + | + | cow |
| 137 | E. coli O26:H11 | + | + | + | + | − | + | + | unknown |
| 138 | E. coli O26:H11 | + | + | + | + | − | + | + | unknown |
| 139 | E. coli O26:NM | + | + | + | + | + | + | + | unknown |
| 140 | E. coli O26:H11 | + | + | + | + | − | + | + | unknown |
| 141 | E. coli O26:H11 | + | + | + | + | + | + | + | unknown |
| 142 | E. coli O26:H11 | + | + | + | + | − | + | + | unknown |
| 143 | E. coli O26 | + | + | + | + | − | + | + | unknown |
| 144 | E. coli O26 | + | + | + | + | − | + | + | unknown |
| 145 | E. coli O26 | + | + | + | + | − | + | + | unknown |
| 146 | E. coli O26 | + | + | + | + | − | + | + | unknown |
| 147 | E. coli O26 | + | + | + | + | − | + | + | unknown |
| 148 | E. coli O26 | + | + | + | + | − | + | + | unknown |
| 149 | E. coli O26 | + | + | + | + | − | + | + | unknown |
| 150 | E. coli O26 | + | + | + | + | − | + | + | unknown |
| 151 | E. coli O26 | + | + | + | + | − | + | + | unknown |
| 152 | E. coli O26A | + | + | + | + | + | + | + | unknown |
| 153 | E. coli O26B | + | + | + | + | + | + | + | unknown |
| 154 | E. coli O45:NM | + | + | + | + | − | + | + | human (F, 77y) |
| 155 | E. coli O45:H2 | + | + | + | + | − | + | + | human (M, 12y) |
| 156 | E. coli O45:H2 | + | + | + | + | − | + | + | human (M, 45y) |
| 157 | E. coli O45:H2 | + | + | + | + | − | + | + | human (F, 38y) |
| 158 | E. coli O45:H2 | + | + | + | + | − | + | + | unknown |
| 159 | E. coli O45:H2 | + | + | + | + | − | + | + | unknown |
| 160 | E. coli O45:H2 | + | + | + | + | − | + | + | unknown |
| 161 | E. coli O45:H2 | + | + | + | + | − | + | + | unknown |
| 162 | E. coli O103:H2 | + | + | + | + | − | + | + | human |
| 163 | E. coli O103:H2(35) | + | + | + | + | − | + | + | ground beef |
| 164 | E. coli O103:H2(35) | + | + | + | + | − | + | + | ground beef |
| 165 | E. coli O103:H2(35) | + | + | + | + | − | + | + | ground beef |
| 166 | E. coli O103:H2(35) | + | + | + | + | − | + | + | ground beef |
| 167 | E. coli O103:H6 | + | + | + | + | − | + | + | human |
| 168 | E. coli O103:H25 | + | + | + | + | − | + | + | human (F, 3y) |
| 169 | E. coli O103:N | + | + | + | + | − | + | + | human |
| 170 | E. coli O103:H2 | + | + | + | + | − | + | + | horse |
| 171 | E. coli O103:H6 | + | + | + | + | − | + | + | human |
| 172 | E. coli O103:NM | + | + | + | + | − | + | + | human |
| 173 | E. coli O103:NM | + | + | + | + | − | + | + | human |
| 174 | E. coli O103:H11 | + | + | + | + | + | + | + | unknown |
| 175 | E. coli O103:H2 | + | + | + | + | − | + | + | unknown |
| 176 | E. coli O103:H2 | + | + | + | + | − | + | + | unknown |
| 177 | E. coli O103:H25 | + | + | + | + | − | + | + | unknown |
| 178 | E. coli O103:H8 | + | + | + | + | − | + | + | unknown |
| 179 | E. coli O103:H2 | + | + | + | + | − | + | + | unknown |
| 180 | E. coli O103:H2 | + | + | + | + | − | + | + | unknown |
| 181 | E. coli O103:H11 | + | + | + | + | − | + | + | unknown |
| 182 | E. coli O103:H2 | + | + | + | + | − | + | + | unknown |
| 183 | E. coli O103:H2 | + | + | + | + | − | + | + | unknown |
| 184 | E. coli O103:H2 | + | + | + | + | − | + | + | unknown |
| 185 | E. coli O103:H2 | + | + | + | + | − | + | + | unknown |
| 186 | E. coli O103 | + | + | + | + | − | + | + | unknown |
| 187 | E. coli O103 | + | + | + | + | + | + | + | unknown |
| 189 | E. coli O111:NM | + | + | + | + | + | + | + | human (M, 67y) |
| 190 | E. coli O111- | + | + | + | + | + | + | + | unknown |
| 191 | E. coli O111:H8 | + | + | + | + | − | + | + | unknown |
| 192 | E. coli O111:H8 | + | + | + | + | + | + | + | human (F, 18y) |
| 193 | E. coli O111:H11 | + | + | + | + | − | + | + | human |
| 194 | E. coli O111:H8 | + | + | + | + | + | + | + | unknown |
| 195 | E. coli O111:H28 | + | + | + | + | − | + | + | human |
| 196 | E. coli O111:NM | + | + | + | + | − | + | + | pig |
| 197 | E. coli O111:H11 | + | + | + | + | − | + | + | cow |
| 198 | E. coli O111:NM | + | + | + | + | + | + | + | unknown |
| 199 | E. coli O111:H11 | + | + | + | + | − | + | + | cow |
| 200 | E. coli O111:NM | + | + | + | + | + | + | + | cow |
| 201 | E. coli O111:NM | + | + | + | + | + | + | + | unknown |
| 202 | E. coli O111:NM | + | + | + | + | + | + | + | cow |
| 203 | E. coli O111:NM | + | + | + | + | − | + | + | unknown |
| 204 | E. coli O111:H8 | + | + | + | + | − | + | + | unknown |
| 205 | E. coli O111:[H8] | + | + | + | + | + | + | + | unknown |
| 206 | E. coli O111:H8 | + | + | + | + | − | + | + | unknown |
| 207 | E. coli O111 | + | + | + | + | + | + | + | unknown |
| 208 | E. coli O111:NM | + | + | + | + | + | + | + | unknown |
| 209 | E. coli O111:NM | + | + | + | + | + | + | + | unknown |
| 210 | E. coli O111:H8 | + | + | + | + | − | + | + | unknown |
| 211 | E. coli O111 | + | + | + | + | − | + | + | unknown |
| 212 | E. coli O111 | + | + | + | + | + | + | + | unknown |
| 213 | E. coli O111 | + | + | + | + | + | + | + | unknown |
| 214 | E. coli O111 | + | + | + | + | + | + | + | unknown |
| 215 | E. coli O121:[H19] | + | + | + | − | + | + | + | human (F, 51y) |
| 216 | E. coli O121:H19 | + | + | + | − | + | + | + | human |
| 217 | E. coli O121 | + | + | + | − | + | + | + | human |
| 218 | E. coli O121:H19 | + | + | + | − | + | + | + | unknown |
| 219 | E. coli O121:H19 | + | + | + | − | + | + | + | unknown |
| 220 | E. coli O121:NM | + | + | + | − | + | + | + | unknown |
| 221 | E. coli O121:H19 | + | + | + | − | + | + | + | unknown |
| 222 | E. coli O121:H19 | + | + | + | − | + | + | + | unknown |
| 223 | E. coli O121:H19 | + | + | + | − | + | + | + | unknown |
| 224 | E. coli O121:H19 | + | + | + | − | + | + | + | unknown |
| 225 | E. coli O145:[28] | + | + | + | − | + | + | + | human |
| 226 | E. coli O145:H28 | + | + | + | + | − | + | + | ground beef |
| 227 | E. coli O145:NM | + | + | + | + | − | + | + | human |
| 228 | E. coli O145:NT | + | + | + | − | + | + | + | human |
| 229 | E. coli O145:+ | + | + | + | − | + | + | + | unknown |
| 230 | E. coli O145 | + | + | + | + | − | + | + | ground beef |
| 231 | E. coli O145:+ | + | + | + | − | + | + | + | food |
| 232 | E. coli O145:NM | + | + | + | + | + | + | + | cow |
| 233 | E. coli O145:NM | + | + | + | + | − | + | + | unknown |
| 234 | E. coli O145:H28 | + | + | + | − | + | + | + | unknown |
| 235 | E. coli O145:NM | + | + | + | − | + | + | + | unknown |
| 236 | E. coli O145:NM | + | + | + | + | − | + | + | unknown |
| 237 | E. coli O145:NM | + | + | + | + | − | + | + | unknown |
| 238 | E. coli O145:NM | + | + | + | + | − | + | + | unknown |
| 239 | E. coli O145:H2 | + | + | + | + | − | + | + | unknown |
| 240 | E. coli O145:H2 | + | + | + | + | − | + | + | unknown |
| 241 | E. coli O145A | + | + | + | + | + | + | + | unknown |
| 242 | E. coli O145B | + | + | + | + | + | + | + | unknown |
| 243 | E. coli O145C | + | + | + | + | + | + | + | unknown |
| 465 | E. coli O113:H21 | + | + | + | + | − | + | + | Canada |
| 466 | E. coli O125:NM | + | + | + | + | − | + | + | USA (N.C.) |
| 467 | E. coli O165:H- | + | + | + | − | + | + | + | beef trim |
| 468 | E. coli O165:H25 | + | + | + | − | + | + | + | unknown |
| 469 | E. coli O5:NM | + | + | + | + | − | + | + | unknown |
| 470 | E. coli O177:[H25] | + | + | + | − | + | + | + | unknown |
| 471 | E. coli unt:H16 | + | + | + | + | − | + | + | human |
| 472 | E. coli unt:H25 | + | + | + | + | − | + | + | unknown |
| 473 | E. coli non-O157:H7 STEC | + | + | + | − | + | + | + | cattle |
| 474 | E. coli unt:H2 | + | + | + | + | − | + | + | beef trim |
| 475 | E. coli O157:H12 | + | + | + | + | + | + | + | pig |
| 476 | E. coli O157:H19 | + | + | + | − | + | + | + | primate |
| 244 | E. coli O157:H43 | − | − | − | − | − | − | − | Unknown |

TABLE 10
Presence of plasmid and chromosomal gene markers in 12 E. coli isolates negative for stx₁ and
stx₂ and containing a top six O serogroup.

| | ecf1 | eae | stx₁ | stx₂ | ehxA | espK | nleB | Z2098 | Z2099 | bfpA |
|---|---|---|---|---|---|---|---|---|---|---|
| E. coli O26 (#476)^{a} | + | + | − | − | + | + | + | + | + | − |
| E. coli O26 (#477)^{a} | + | + | − | − | + | + | + | + | + | − |
| E. coli O26 (#480)^{a} | + | + | − | − | + | + | + | + | + | − |
| E. coli O103 (#481)^{a} | + | + | − | − | + | + | + | − | − | − |
| E. coli O103 (#482)^{a} | + | + | − | − | + | + | + | + | + | − |
| E. coli O103 (#483)^{a} | + | + | − | − | + | + | + | + | + | − |
| E. coli O103 (#484)^{a} | + | + | − | − | + | + | + | + | + | − |
| E. coli O145 (#485)^{a} | + | + | − | − | + | + | + | + | + | − |
| E. coli O145 (#486)^{a} | + | + | − | − | + | + | + | + | + | − |
| E. coli O145 (#487)^{a} | + | + | − | − | + | + | + | + | + | − |
| E. coli O145 (#488)^{a} | + | + | − | − | + | + | + | + | + | − |
| E. coli O145 (#489)^{a} | + | + | − | − | + | + | + | + | + | − |
| E. coli O157:H7 | + | + | + | + | + | + | + | + | + | − |
| Control | | | | | | | | | | |
| E. coli O55:H6 | − | + | − | − | − | − | + | − | − | + |
| Control | | | | | | | | | | |

^{a}E. coli isolate number based on Table 4b

### Screening of Ground Beef Samples for Ecf Specificity

Two beef studies were conducted in order to investigate the utility of the ecf1 gene to be used as a single marker for non-O157 STEC detection in primary meat enrichments. In Table 6 we summarize screening results from 1065 enriched ground beef samples from a commercial ground beef producer over the period of January to June 2012 (Study I). Each enrichment bag was screened for ecf1, stx1, stx2, eae and ehxA. All stx/eae positive samples in addition to ecf1 positive samples were then screened for each of the six most frequent non-O157 STEC O serogroups O26, O45, O103, O111, O121, and O145. The prevalence of stx, eae and ehxA in this study was 19%, 14.6% and 14.6%, respectively. As summarized in Table 11, 6.5% of the samples were ecf1 positive and 4.0% were positive for ecf1 as well as for one of the six most frequent non-O157 STEC O serogroups. In contrast, 7.8% of the ground beef samples were positive for both FSIS recommended STEC markers stx and eae and 5.0% of the samples were positive for stx, eae and one of the six most frequent non-O157 STEC. The most prevalent O serogroups in ecf1 positive ground beef samples were O103 (61.4%), O26 (45.5%), and O45 (31.8%). Only 11.4% ecf1 positive samples were positive for O121 and no O111 or O145 serogroups were detected. The prevalence of O serogroups O103, O26, and O45 in stx/eae positive ground beef samples was 40.2% (O103), 23.2% (O26), and 19.5% (O45), respectively. Out of all 44 ecf1 positive ground beef samples which were also positive for one of the six most frequent non-O157 STEC O serogroups 45.5% (n=20) contained two or more of the six most frequent non-O157 STEC O serogroups detected by PCR. The frequency of samples containing two or more O serogroups detected by the FSIS stx/eae method was 40.4% (n=21) indicating multiple E. coli O serogroups within the same enrichment bag.

TABLE 11
Positivity of ecf1 and other virulence markers in 1065 enriched
beef samples obtained from a commercial ground beef producer

Positivity of Beef Samples
(n = 1065) from Meat Processor

| | N | % |
|---|---|---|
| Ecf | 69 | 6.5 |
| stx + eae | 83 | 7.8 |
| Stx | 202 | 19 |
| Eae | 155 | 14.6 |
| ehxA | 155 | 14.6 |
| ecf + Big 6 O type | 43 | 4.0 |
| stx + eae + O Big 6 O type | 53 | 5.0 |

In Table 12 we summarize the screening results from 1097 beef trim and ground beef samples obtained from an independent certified testing laboratory over the period of August 2012 to January 2013 (Study II). Eighty-percent of these samples were beef trim samples and 20% were ground beef samples. Each enriched beef sample was screened for ecf1 and eae. If a sample was positive for either ecf1 or eae it was further screened with oligonucleotides specific for stx1, stx2, ehxA and the six most frequent non-O157 STEC O serogroup genes. As summarized in Table 12, 3.4% (36 beef trim samples and 1 ground beef sample) of the enriched beef samples were ecf1 positive and 1.1% (12 beef trim samples) were positive for ecf1 as well as for one of the six most frequent non-O157 STEC O serogroups. The most prevalent O serogroups were O103 and O45 (33% each) followed by O26 and O145 (16.7% each). No O111 or O121 serogroups were detected. In contrast, 4.3% of the beef samples were positive for the FSIS recommended STEC markers stx and eae and 1.1% (12 beef trim samples) were positive for stx, eae and one of the six most frequent non-O157 STEC. The most prevalent O serogroups were O103 and O45 (33% each) followed by O26 and O145 (16.7% each). No O111 or O121 serogroups were detected. Out of the 12 ecf1 positive beef trim samples which were also positive for stx and eae and one of the six most frequent non-O157 STEC O serogroups only 8% contained two or more six most frequent non-O157 STEC O serogroups indicating a low level of multiple E. coli O serogroups within the same enrichment bag.

TABLE 12
Positivity of ecf-1 and other virulence
markers in 1097 enriched beef samples

Positivity of Beef Samples
(n = 1097)

| | n | % |
|---|---|---|
| Ecf | 37 | 3.4 |
| stx + eae | 47 | 4.3 |
| ecf + Big 6 O type | 12 | 1.1 |
| stx + eae + O Big 6 O type | 12 | 1.1 |

Since the FSIS screening method detects stx and eae genes that may reside in different organisms potential false positive rates for this screening method may occur. To estimate the potential false positive rate of the eae/stx based-method we examined each stx and eae positive but ecf1 negative enriched meat sample in Study I for the detection of the specific eae-positive STEC markers, Z2098 and Z2099. We observed 22 samples which were positive for stx and eae but negative for ecf1 and out of these samples 15 were six most frequent non-O157 STEC (Table 13). None of these 22 enriched beef samples were positive for the markers Z2098 or Z2099 (Table 13).

TABLE 13
Samples with discrepant results by the stx/eae-method and the ecf1-detection method

| | Total number of | | | | | | | | | |
|---|---|---|---|---|---|---|---|---|---|---|
| | discrepant | | | | Gene Markers | | | | | |
| Study I (n-1065) | samples | | | n | ecf1 | stx^{a} | eae | ehxA | Z2098 | Z2099 |
| stx/eae-method | 22 | | | 7 | − | + | + | − | − | − |
| | | | | 10 | − | + | + | − | − | − |
| | | Top six | {open oversize brace} | | | | | | | |
| | | non-O157 STEC | | 5 | − | + | + | + | − | − |
| ecf1-detection method | 8 | Top six | | 3 | + | − | + | + | + | + |
| | | non-O157 STEC | | 5 | + | − | + | + | + | + |

^{a}Positive if stx₁ and/or stx₂ positive

To identify potential false positive results by the ecf1 detection assay in Study I we examined each ecf1 positive sample for the absence of the eae or stx genes. None of the ecf1 positive enrichment bags were missing eae while 8 were missing stx. All eight samples were positive for both Z2098 and Z2099 markers, arguing that these samples are non-O157 STEC that have lost stx genes (Table 13).

Based on the analysis of 501 E. coli isolates from various human and food sources, all E. coli isolates that contained stx, eae and ehxA were found to be ecf positive demonstrating that ecf is a very accurate surrogate marker for EHEC strains. These isolates included O157:H7, O157:H7:NM strains containing stx, Big 6 STEC's, and non-Big 6 STEC's. Although the majority of our work targeted the ecf-1 gene, analysis of 253 E. coli isolates revealed the same results for ecf-3 and ecf-4 genes. In contrast, not all ecf positive strains harbored eae, stx and ehxA. We identified ecf positive E. coli isolates as well as ecf positive enrichment meat samples that lacked stx. Because the E. coli isolates contained eae they could be EPECs containing ecf. However, none of the 12 isolates contained the BfpA gene found in typical EPEC's and all of them except one contained genes found in EHEC strains including the EHEC-specific Z2098 and Z2099 genes. The one isolate lacking the Z2098 and Z2099 genes contained the EHEC genes ecf, eae, ehxA, espK and nleB and is therefore also likely to be an EHEC strain that is not detected by Z2098 and Z2099. Because the E. coli strains containing ecf but lacking stx are EHEC's, we conclude that they have lost stx. Loss of stx genes during passage in the laboratory or in response to immune attack has been well documented.

Ecf and ehxA are located on what has been termed the large EHEC hemolysin plasmid. Z2098 and Z2099 appear to be the most specific chromosomal markers for EHEC. Based on our results, any E. coli strain that contains stx, eae and ehxA is an EHEC strain. Because ecf and ehxA both reside on the same plasmid EHEC's will be ecf positive. We have shown that stx and the large EHEC plasmid as well as portions of the EHEC plasmid can be lost from ecf positive strains. Therefore all E. coli containing stx, eae and ehxA are EHECs while all EHECs may not contain stx or an intact large EHEC plasmid.

Current regulations by the FSIS require testing of beef trim for the non-O157:H7 Big 6 STEC's (O26, O45, O103, O111, O121, O145). These 6 STECs were chosen not because they are the most virulent but because together they represent 70-80% of the STECs known to cause disease in humans. Our results reveal that all non-O157:H7 STECs other than the Big 6 isolates are accurately detected by ecf. Thus ecf is capable of detecting STECs known to cause human disease that are missed by current FSIS guidelines. Our analysis of STEC strains isolated from ground beef samples across the United States reveals that these STEC's are in the beef supply. Of the 11 ecf positive isolates from ground beef, 3 were not non-O157:H7 Big 6 STECs and one of these strains, O165, has been shown to cause HUS.

To investigate the utility of ecf to detect STECs in ground beef and beef trim samples, we carried out studies testing samples from a ground beef processor and an independent certified testing laboratory in the United States over a period of a year. We compared results we obtained with the ecf marker with those obtained by the current FSIS screening method that detects eae and stx and which is used in current commercial assays. In the first study both ecf and the FSIS method found a high level, 4.0% and 5.0%, respectively, of non-O157:H7 Big 6 STECs in ground beef enrichments. The overall level of all non-O157:H7 STECs was higher at 6.5% and 7.8% for ecf and the FSIS method, respectively. Because the FSIS method detects 2 genes that may reside in different organisms false positive rates for this method are expected to be high owing to co-contaminating bacteria and higher than detection by the ecf gene. In this study there was a significant amount of co-contamination as 43% of enrichment bags that were positive for a non-O157:H7 Big 6 STEC were positive for 2 or more of these STECs. To estimate the false positive rate of the FSIS method we examined each FSIS positive enrichment sample for the absence of ecf1. In the first beef study there were 22 such samples. To determine whether these samples had plasmid loss we screened them for two EHEC markers, Z2098 and Z2099. Delannoy et al found the Z2098 and Z2099 gene markers had a detection range of 89.6-95.5% for STEC with top six O serogroups, and a range of 67.6-96.8% for emerging STEC with other O serogroups. Although the Z2098 and Z2099 markers are not associated with all eae-positive STEC, three O serogroups (O26, O103 and O145) previously demonstrated 100% detection using the Z2098 marker. Twelve out of the 22 samples that tested positive for eae and stx but negative for ecf1 and Z2098 contained these O serogroups, thus indicating eae and stx genes resided in different organisms in these twelve samples. We estimated an additional 6 to 9 samples out of the remaining 10 samples that tested positive for eae and stx but negative for ecf1 likely resulted from co-contamination based on the prevalence of Z2098 and Z2099 markers in STEC containing other O serogroups. Overall, we estimated 1.7-2.0% of samples in Study I (18-21/1065) led to false positive results using the eae/stx screening method compared to 0.8% (8/1065) using the ecf1-detection method. Estimation of false positive rates among STEC containing the top six O serogroups also revealed a lower rate using the ecf1 detection method (5/1065, 0.5%) compared to the eae/stx screening method (12/1065, 1.1%).

Although a method using ecf1 as a STEC marker does not suffer from false positive test results arising from co-contaminated samples it could potentially have false positive results arising from EHEC with a loss of stx. Of the 43 non-O157:H7 Big 6 STEC detected by ecf1 five were stx minus and thus false positive test results. A total of 69 samples were positive for any non-O157:H7 STEC serotype but eight were stx minus and therefore false positive test results. Thus the percentage of false positive samples using ecf1 as an EHEC marker is half the value observed by the FSIS method. This suggests that the majority of false positive samples detected by ecf1 could be eliminated by a stx confirmation whereas culturing of the E. coli isolate is the only method that could reduce the false positive rate of the FSIS method. The only positive samples missed by the use of a combination of ecf1 and stx would be those arising from enrichment bags co-contaminated with microorganisms harboring, separately, each gene.

In a second beef study the level of STEC's were lower than in the first study. Whereas in the first study the level of total non-O157:H7 STEC's was 7.8% and 6.5%, respectively for the FSIS and method disclosed herein in the second study they were only 4.3% and 3.4%, respectively. Furthermore, in the second study the level of non-O157:H7 Big 6 STECs was only 1.1% for both the FSIS and use of ecf1 as is disclosed herein. One possible reason for the higher level of Big 6 STECs in the first study is the higher percentage of enrichment bags co-contaminated by two or more Big 6 STECs in the first versus the second study. Since the presence of a Big 6 O serotype is determined by detecting an O type gene an enrichment bag could have one bacterium contribute the stx, eae or ecf1 signal and another bacteria contribute the O type gene leading to false positive identification of a Big 6 STEC. In this study the number of putative false positives obtained by the FSIS and the disclosed methodology for the Big 6 STECs was 0%. The total number of putative false positives obtained by the FSIS and the disclosed methodology in the second beef study could not be evaluated due to insufficient DNA for the analysis of additional virulence gene markers.

In sum, these results described here demonstrate the ecf1 gene is an accurate surrogate marker for the detection of stx and eae and exhA positive non-O157 STEC in ground beef and beef trim samples. The ecf1 detection assay utilizes a single gene with the potential of lowering presumptive false positive rates compared to methods that detect eae and stx genes. Furthermore, the ecf1 detection assay is capable of identifying STEC with O serogroups other than O26, O45, O103, O111, O121, and O145 which are known to cause human disease and are missed by current FSIS guidelines.

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

What is claimed is:

1. A method for testing a sample for the presence of a pathogenic Escherichia coli, said method comprising detecting the presence of (i) ecf and (ii) wzx and/or stx in the sample, wherein detection of (i) ecf and (ii) wzx and/or stx in said sample is taken as an indication that said sample includes said pathogenic E. coli.

2. The method of claim 1, wherein said detecting the presence of ecf comprises detecting the presence of a nucleic acid encoding the ecf operon, or a portion thereof.

3. The method of claim 1, wherein said detecting the presence of ecf comprises detecting the presence of a nucleic acid encoding ecf-1, or a portion thereof.

4. The method of claim 1, wherein said detecting the presence of ecf comprises detecting the presence of a nucleic acid encoding ecf-2, or a portion thereof.

5. The method of claim 1, wherein said detecting the presence of ecf comprises detecting the presence of a nucleic acid encoding ecf-3, or a portion thereof.

6. The method of claim 1, wherein said detecting the presence of ecf comprises detecting the presence of a nucleic acid encoding ecf-4, or a portion thereof.

7. The method of claim 1, wherein said detecting the presence of ecf comprises detecting the presence of ecf 1 polypeptide.

8. The method of claim 1, wherein said detecting the presence of ecf comprises detecting the presence of ecf 2 polypeptide.

9. The method of claim 1, wherein said detecting the presence of ecf comprises detecting the presence of ecf 3 polypeptide.

10. The method of claim 1, wherein said detecting the presence of ecf comprises detecting the presence of ecf 4 polypeptide.

11. The method of any of claims 1-10, wherein said detecting the presence of wzx comprises detecting the presence of a nucleic acid encoding wzx.

12. The method of any of claims 1-10, wherein said detecting the presence of wzx comprises detecting the presence of wzx polypeptide.

13. The method of any of claims 1-12, wherein said detecting the presence of stx comprises detecting the presence of a nucleic acid encoding stx1.

14. The method of any of claims 1-12, wherein said detecting the presence of stx comprises detecting the presence of stx1 polypeptide.

15. The method of any of claims 1-12, wherein said detecting the presence of stx comprises detecting the presence of a nucleic acid encoding is stx2.

16. The method of any of claims 1-12, wherein said detecting the presence of stx comprises detecting the presence of stx2 polypeptide.

17. The method of any of claims 1-16, wherein detection of ecf and wzx is taken as an indication of the presence of E. coli O157:H7.

18. The method of any of claims 1-16, wherein detection ecf and the absence of wzx is taken as an indication of the presence of non-O157:H7 shiga toxin (stx)-containing E. coli (STEC).

19. The method of any of claims 1-16, wherein detection of ecf and stx is taken as an indication of the presence of enterohemorrhagic Escherichia coli (EHEC).

20. The method of any of claims 1-19, wherein said sample is obtained following enrichment of high fat ground beef, beef trim, or produce (such as fruits such as grapes, apples, peaches, or strawberries and/or vegetables such as lettuce, spinach, radishes and alfalfa sprouts).

21. The method of any of claim 1-6, 11, 13, or 15, wherein said detecting comprises contacting the sample with an oligonucleotide that hybridizes to a portion of a nucleic acid encoding the ecf operon, a nucleic acid encoding wzx, a nucleic acid encoding stx1, or a nucleic acid encoding stx2.

22. The method of claim 21, wherein said detecting comprises a hybridization assay selected from the group consisting of a Transcription Mediated Amplification (TMA) reaction, a Nucleic Acid Sequence-Based Amplification (NASBA) reaction, a Polymerase Chain Reaction (PCR) reaction, a hybridization protection assay, or a non-amplified hybridization reaction.

23. The method of claim 21 or 22, wherein the hybridizing oligonucleotide comprises a detectable label.

24. The method of any of claim 1, 7-10, 12, 14, or 16, wherein detecting comprises a polypeptide detection assay.

25. The method of claim 24, wherein said polypeptide detection assay is an immunoassay.

26. The method of claim 24 or 25, wherein said detecting comprises contacting the sample with a molecule that specifically binds to a polypeptide selected from the group consisting of ecf1, ecf2, ecf3, ecf4, wzx, stx1, and stx2.

27. The method of claim 26, wherein said molecule comprises a detectable label.

28. The method of claim 26 or 27, wherein said molecule comprises an antibody or fragment thereof.

29. The method of any of claims 1-28, wherein said detecting of (i) and detecting of (ii) are performed in a single reaction mixture.

30. A composition comprising (i) a first oligonucleotide that specifically hybridizes to a nucleic acid encoding the ecf operon, or portion thereof, and (ii) a second oligonucleotide that specifically hybridizes to a nucleic acid encoding wzx, stx1, or stx2.

31. The composition of claim 30, wherein said first oligonucleotide specifically hybridizes to a nucleic acid encoding ecf-1, or a portion thereof.

32. The composition of claim 30, wherein said first oligonucleotide specifically hybridizes to a nucleic acid encoding ecf-2, or a portion thereof.

33. The composition of claim 30, wherein said first oligonucleotide specifically hybridizes to a nucleic acid encoding ecf-3, or a portion thereof.

34. The composition of claim 30, wherein said first oligonucleotide specifically hybridizes to a nucleic acid encoding ecf-4, or a portion thereof.

35. The composition of claim 30, wherein said second oligonucleotide specifically hybridizes to a nucleic acid encoding wzx.

36. The composition of claim 30, wherein said second oligonucleotide specifically hybridizes to a nucleic acid encoding stx1 or stx2.

37. The composition of any of claims 30-36, wherein said first and/or said second oligonucleotides are detectably labeled.

38. The composition of any of claims 30-37, further comprising primers for performing a Transcription Mediated Amplification (TMA) reaction, a Nucleic Acid Sequence-Based Amplification (NASBA) reaction and/or a Polymerase Chain Reaction (PCR) reaction.

39. A composition comprising (i) a first amplicon produced by a method of amplifying a nucleic acid encoding the ecf operon and (ii) a second amplicon produced by a method of amplifying a nucleic acid encoding wzx, stx1, or stx2.

40. The composition of claim 39, wherein said first amplicon is produced by a method of amplifying a nucleic acid encoding ecf1.

41. The composition of claim 39, wherein said first amplicon is produced by a method of amplifying a nucleic acid encoding ecf2.

42. The composition of claim 39, wherein said first amplicon is produced by a method of amplifying a nucleic acid encoding ecf3.

43. The composition of claim 39, wherein said first amplicon is produced by a method of amplifying a nucleic acid encoding ecf4.

44. The composition of claim 39, wherein said second amplicon is produced by a method of amplifying a nucleic acid encoding wzx.

45. The composition of claim 39, wherein said second amplicon is produced by a method of amplifying a nucleic acid encoding stx1 or stx2.

46. The composition of any of claims 39-45, wherein method of amplifying the nucleic acid is selected from the group consisting of Transcription Mediated Amplification (TMA) reaction, a Nucleic Acid Sequence-Based Amplification (NASBA) reaction and a Polymerase Chain Reaction (PCR) reaction.

47. The composition of any of claims 39-46, wherein said first and/or second amplicon is detectably labeled.

48. A method for producing a packaged lot of meat free of a pathogenic Escherichia coli adulterant, said method comprising the steps of
a) providing a sample obtained from a lot of meat;
b) testing said sample for the presence of (i) ecf and (ii) wzx and/or stx in the sample, wherein absence of (i) ecf and (ii) wzx and/or stx in said sample is taken as an indication that said sample is free of pathogenic E. coli adulterant; and
c) packaging meat identified as free of the pathogenic E. coli adulterant.

49. The method of claim 48, wherein said detecting the presence of ecf comprises detecting the presence of a nucleic acid encoding the ecf operon, or a portion thereof.

50. The method of claim 48, wherein said detecting the presence of ecf comprises detecting the presence of a nucleic acid encoding ecf-1, or a portion thereof.

51. The method of claim 48, wherein said detecting the presence of ecf comprises detecting the presence of a nucleic acid encoding ecf-2, or a portion thereof.

52. The method of claim 48, wherein said detecting the presence of ecf comprises detecting the presence of a nucleic acid encoding ecf-3, or a portion thereof.

53. The method of claim 48, wherein said detecting the presence of ecf comprises detecting the presence of a nucleic acid encoding ecf-4, or a portion thereof.

54. The method of claim 48, wherein said detecting the presence of ecf comprises detecting the presence of ecf 1 polypeptide.

55. The method of claim 48, wherein said detecting the presence of ecf comprises detecting the presence of ecf 2 polypeptide.

56. The method of claim 48, wherein said detecting the presence of ecf comprises detecting the presence of ecf 3 polypeptide.

57. The method of claim 48, wherein said detecting the presence of ecf comprises detecting the presence of ecf 4 polypeptide.

58. The method of any of claims 48-57, wherein said detecting the presence of wzx comprises detecting the presence of a nucleic acid encoding wzx.

59. The method of any of claims 48-57, wherein said detecting the presence of wzx comprises detecting the presence of wzx polypeptide.

60. The method of any of claims 48-59, wherein said detecting the presence of stx comprises detecting the presence of a nucleic acid encoding stx1.

61. The method of any of claims 48-59, wherein said detecting the presence of stx comprises detecting the presence of stx1 polypeptide.

62. The method of any of claims 48-59, wherein said detecting the presence of stx comprises detecting the presence of a nucleic acid encoding is stx2.

63. The method of any of claims 48-59, wherein said detecting the presence of stx comprises detecting the presence of stx2 polypeptide.

64. The method of any of claims 48-63, wherein detection of ecf and wzx is taken as an indication of the presence of E. coli O157:H7.

65. The method of any of claims 48-63, wherein detection ecf and the absence of wzx is taken as an indication of the presence of non-O157:H7 shiga toxin (stx)-containing E. coli (STEC).

66. The method of any of claims 48-63, wherein detection of ecf and stx is taken as an indication of the presence of enterohemorrhagic Escherichia coli (EHEC).

67. The method of any of claims 48-66, wherein said sample is obtained following enrichment of a meat sample.

68. The method of any of claim 48-53, 58, 60, or 62, wherein said detecting comprises contacting the sample with an oligonucleotide that hybridizes to a portion of a nucleic acid encoding the ecf operon, a nucleic acid encoding wzx, a nucleic acid encoding stx1, or a nucleic acid encoding stx2.

69. The method of claim 68, wherein said detecting comprises a hybridization assay selected from the group consisting of a Transcription Mediated Amplification (TMA) reaction, a Nucleic Acid Sequence-Based Amplification (NASBA) reaction, a Polymerase Chain Reaction (PCR) reaction, a hybridization protection assay, or a non-amplified hybridization reaction.

70. The method of claim 68 or 69, wherein the hybridizing oligonucleotide comprises a detectable label.

71. The method of any of claim 48, 54-57, 59, 61, or 64, wherein detecting comprises a polypeptide detection assay.

72. The method of claim 71, wherein said polypeptide detection assay is an immunoassay.

73. The method of claim 71 or 72, wherein said detecting comprises contacting the sample with a molecule that specifically binds to a polypeptide selected from the group consisting of ecf1, ecf2, ecf3, ecf4, wzx, stx1, and stx2.

74. The method of claim 73, wherein said molecule comprises a detectable label.

75. The method of claim 73 or 74, wherein said molecule comprises an antibody or fragment thereof.

76. The method of any of claims 48-75, further comprising shipping the packaged meat.

77. The method of claim 48-76, wherein said packaging comprises a carton, container, plastic wrap, or a meat tray wrapped with plastic.

78. The method of any of claims 48-77, wherein said lot of meat comprises raw ground beef, high fat ground beef, or raw ground beef components (for example, beef and veal bulk packed manufacturing trimmings and other beef and veal components such as primal cuts, sub primal cuts, head meat, cheek meat, esophagus meat, heart, and advanced meat recovery product intended for grinding).

79. The method of any of claims 48-78, wherein said sample provided for enrichment is about 200 g to about 500 g.

80. The method of claim 79, wherein said sample is about 325 g to about 375 g.

81. The method of any of claims 48-80, wherein said detecting of (i) and detecting of (ii) are performed in a single reaction mixture.

82. A method for producing a lot of produce free of a pathogenic Escherichia coli adulterant, said method comprising the steps of
a) providing a sample obtained from a lot of produce;
b) testing for the presence of (i) ecf and (ii) wzx and/or stx in the sample, wherein absence of (i) ecf and (ii) wzx and/or stx in said sample is taken as an indication that said sample is free of pathogenic E. coli adulterant; and
c) packaging produce identified as free of the pathogenic E. coli adulterant.

83. The method of claim 82, wherein said detecting the presence of ecf comprises detecting the presence of a nucleic acid encoding the ecf operon, or a portion thereof.

84. The method of claim 82, wherein said detecting the presence of ecf comprises detecting the presence of a nucleic acid encoding ecf-1, or a portion thereof.

85. The method of claim 82, wherein said detecting the presence of ecf comprises detecting the presence of a nucleic acid encoding ecf-2, or a portion thereof.

86. The method of claim 82, wherein said detecting the presence of ecf comprises detecting the presence of a nucleic acid encoding ecf-3, or a portion thereof.

87. The method of claim 82, wherein said detecting the presence of ecf comprises detecting the presence of a nucleic acid encoding ecf-4, or a portion thereof.

88. The method of claim 82, wherein said detecting the presence of ecf comprises detecting the presence of ecf 1 polypeptide.

89. The method of claim 82, wherein said detecting the presence of ecf comprises detecting the presence of ecf 2 polypeptide.

90. The method of claim 82, wherein said detecting the presence of ecf comprises detecting the presence of ecf 3 polypeptide.

91. The method of claim 82, wherein said detecting the presence of ecf comprises detecting the presence of ecf 4 polypeptide.

92. The method of any of claims 82-91, wherein said detecting the presence of wzx comprises detecting the presence of a nucleic acid encoding wzx.

93. The method of any of claims 82-91, wherein said detecting the presence of wzx comprises detecting the presence of wzx polypeptide.

94. The method of any of claims 82-93, wherein said detecting the presence of stx comprises detecting the presence of a nucleic acid encoding stx1.

95. The method of any of claims 82-93, wherein said detecting the presence of stx comprises detecting the presence of stx1 polypeptide.

96. The method of any of claims 82-93, wherein said detecting the presence of stx comprises detecting the presence of a nucleic acid encoding is stx2.

97. The method of any of claims 82-93, wherein said detecting the presence of stx comprises detecting the presence of stx2 polypeptide.

98. The method of any of claims 82-97, wherein detection of ecf and wzx is taken as an indication of the presence of E. coli O157:H7.

99. The method of any of claims 82-97, wherein detection ecf and the absence of wzx is taken as an indication of the presence of non-O157:H7 shiga toxin (stx)-containing E. coli (STEC).

100. The method of any of claims 82-97, wherein detection of ecf and stx is taken as an indication of the presence of enterohemorrhagic E. coli (EHEC).

101. The method of any of claim 82-87, 92, 94, or 96, wherein said detecting comprises contacting the sample with an oligonucleotide that hybridizes to a portion of a nucleic acid encoding the ecf operon, a nucleic acid encoding wzx, a nucleic acid encoding stx1, or a nucleic acid encoding stx2.

102. The method of claim 101, wherein said detecting comprises a hybridization assay selected from the group consisting of a Transcription Mediated Amplification (TMA) reaction, a Nucleic Acid Sequence-Based Amplification (NASBA) reaction, a Polymerase Chain Reaction (PCR) reaction, a hybridization protection assay, or a non-amplified hybridization reaction.

103. The method of claim 101 or 102, wherein the hybridizing oligonucleotide comprises a detectable label.

104. The method of any of claim 82, 88-91, 93, 95, or 97, wherein detecting comprises a polypeptide detection assay.

105. The method of claim 104, wherein said polypeptide detection assay is an immunoassay.

106. The method of claim 104 or 105, wherein said detecting comprises contacting the sample with a molecule that specifically binds to a polypeptide selected from the group consisting of ecf1, ecf2, ecf3, ecf4, wzx, stx1, and stx2.

107. The method of claim 106, wherein said molecule comprises a detectable label.

108. The method of claim 106 or 107, wherein said molecule comprises an antibody or fragment thereof.

109. The method of any of claims 82-108, further comprising shipping the packaged produce.

110. The method of any of claims 82-109, wherein said packaging comprises a carton, container, plastic wrap, or a produce tray wrapped with plastic.

111. The method of any of claims 82-110, wherein said detecting of (i) and detecting of (ii) are performed in a single reaction mixture.

112. The method of any of claims 82-111, wherein said lot of produce comprises fruit or vegetables (such as lettuce, spinach, cabbage, celery, cilantro, coriander, cress sprouts, radishes, or alfalfa sprouts).

113. The method of any of claims 82-112, wherein said sample obtained from a lot of produce is about 200 g to about 500 g.

114. The method of claim 113, wherein said sample is about 325 g to about 375 g.


SEQUENCE LISTING (211 sequences)

SEQ ID NO: 1 - DNA, 25 bp, Artificial Sequence
  Description of Artificial Sequence Synthetic primer
cccttatgaagagccagtactgaag

SEQ ID NO: 2 - DNA, 24 bp, Artificial Sequence
  Description of Artificial Sequence Synthetic primer
attacgcatagggcgtatcagcac

SEQ ID NO: 3 - DNA, 24 bp, Artificial Sequence
  Description of Artificial Sequence Synthetic primer
tgcaaggcatcttcccgtactgat

SEQ ID NO: 4 - DNA, 24 bp, Artificial Sequence
  Description of Artificial Sequence Synthetic primer
tctgcgagccacttcatctgttca

SEQ ID NO: 5 - DNA, 24 bp, Artificial Sequence
  Description of Artificial Sequence Synthetic primer
agcaggaatattctcaccgcgact

SEQ ID NO: 6 - DNA, 24 bp, Artificial Sequence
  Description of Artificial Sequence Synthetic primer
acagacaacctgtcccagcgttta

SEQ ID NO: 7 - DNA, 24 bp, Artificial Sequence
  Description of Artificial Sequence Synthetic primer
ttcctttgccatggcggagaattg

SEQ ID NO: 8 - DNA, 24 bp, Artificial Sequence
  Description of Artificial Sequence Synthetic primer
agcggctcctgtctgattaacgat

SEQ ID NO: 9 - DNA, 24 bp, Artificial Sequence
  Description of Artificial Sequence Synthetic primer
tgatcatcgtgcatctgctgggta

SEQ ID NO: 10 - DNA, 24 bp, Artificial Sequence
  Description of Artificial Sequence Synthetic primer
atgccctgtaatgccatcaaaccg

SEQ ID NO: 11 - DNA, 24 bp, Artificial Sequence
  Description of Artificial Sequence Synthetic primer
tgtacactgttccgttcctgctgt

SEQ ID NO: 12 - DNA, 24 bp, Artificial Sequence
  Description of Artificial Sequence Synthetic primer
tccctgaattgcggattcaccaga

SEQ ID NO: 13 - DNA, 24 bp, Artificial Sequence
  Description of Artificial Sequence Synthetic primer
acgctggaatggtctggagattgt

SEQ ID NO: 14 - DNA, 24 bp, Artificial Sequence
  Description of Artificial Sequence Synthetic primer
atccaccaccggatttctctggtt

SEQ ID NO: 15 - DNA, 24 bp, Artificial Sequence
  Description of Artificial Sequence Synthetic primer
aactttaccggttatcggacggct

SEQ ID NO: 16 - DNA, 24 bp, Artificial Sequence
  Description of Artificial Sequence Synthetic primer
tgctcaggatgtggacgaacgaaa

SEQ ID NO: 17 - DNA, 24 bp, Artificial Sequence
  Description of Artificial Sequence Synthetic primer
tggtaccaccttctgctgtactct

SEQ ID NO: 18 - DNA, 24 bp, Artificial Sequence
  Description of Artificial Sequence Synthetic primer
tacctgtccacgtcatccagtaac

SEQ ID NO: 19 - DNA, 25 bp, Artificial Sequence
  Description of Artificial Sequence Synthetic primer
ttaattttgatgccagccaggttgg

SEQ ID NO: 20 - DNA, 26 bp, Artificial Sequence
  Description of Artificial Sequence Synthetic primer
gctagattctgacgttattgctggtc

SEQ ID NO: 21 - DNA, 26 bp, Artificial Sequence
  Description of Artificial Sequence Synthetic primer
aggcaagtaaaacggaatgtccctgc

SEQ ID NO: 22 - DNA, 26 bp, Artificial Sequence
  Description of Artificial Sequence Synthetic primer
tatgttgaatgcaaggcatcttcccg

SEQ ID NO: 23 - DNA, 26 bp, Artificial Sequence
  Description of Artificial Sequence Synthetic primer
gctctttcgcatttaatccagtggga

SEQ ID NO: 24 - DNA, 26 bp, Artificial Sequence
  Description of Artificial Sequence Synthetic primer
tacagcggaacgaatggaatacggga

SEQ ID NO: 25 - DNA, 21 bp, Artificial Sequence
  Description of Artificial Sequence Synthetic primer
atctccaaggcggcaacgaaa

SEQ ID NO: 26 - DNA, 30 bp, Artificial Sequence
  Description of Artificial Sequence Synthetic primer
cagaaggttatgaagttgagttcattccag

SEQ ID NO: 27 - DNA, 23 bp, Artificial Sequence
  Description of Artificial Sequence Synthetic primer
ataaatcgccattcgttgactac

SEQ ID NO: 28 - DNA, 21 bp, Artificial Sequence
  Description of Artificial Sequence Synthetic primer
agaacgcccactgagatcatc

SEQ ID NO: 29 - DNA, 21 bp, Artificial Sequence
  Description of Artificial Sequence Synthetic primer
ggcactgtctgaaactgctcc

SEQ ID NO: 30 - DNA, 22 bp, Artificial Sequence
  Description of Artificial Sequence Synthetic primer
tcgccagttatctgacattctg

SEQ ID NO: 31 - DNA, 26 bp, Artificial Sequence
  Description of Artificial Sequence Synthetic primer
cattgatcaggatttttctggtgata

SEQ ID NO: 32 - DNA, 21 bp, Artificial Sequence
  Description of Artificial Sequence Synthetic primer
ctcatgcggaaatagccgtta

SEQ ID NO: 33 - DNA, 24 bp, Artificial Sequence
  Description of Artificial Sequence Synthetic primer
ctggactcaacgtggatttcatca

SEQ ID NO: 34 - DNA, 27 bp, Artificial Sequence
  Description of Artificial Sequence Synthetic primer
acctaacgctaacaaagctaaatgaag

SEQ ID NO: 35 - DNA, 21 bp, Artificial Sequence
  Description of Artificial Sequence Synthetic primer
gtgtcagtagggaagcgaaca

SEQ ID NO: 36 - DNA, 19 bp, Artificial Sequence
  Description of Artificial Sequence Synthetic primer
atcatgttttccgccaatg

SEQ ID NO: 37 - DNA, 24 bp, Artificial Sequence
  Description of Artificial Sequence Synthetic primer
tgcgtgtggcaaaaatttaaagat

SEQ ID NO: 38 - DNA, 24 bp, Artificial Sequence
  Description of Artificial Sequence Synthetic primer
gttgccaatcaatcatgccagaag

SEQ ID NO: 39 - DNA, 24 bp, Artificial Sequence
  Description of Artificial Sequence Synthetic primer
agttaggcactctggcaacatgga

SEQ ID NO: 40 - DNA, 24 bp, Artificial Sequence
  Description of Artificial Sequence Synthetic primer
atgagcatctgcataagcagccca

SEQ ID NO: 41 - DNA, 27 bp, Artificial Sequence
  Description of Artificial Sequence Synthetic primer
cctctcaattgtcagggaaattagcgt

SEQ ID NO: 42 - DNA, 24 bp, Artificial Sequence
  Description of Artificial Sequence Synthetic primer
tgttaatggttgaaccgacggcag

SEQ ID NO: 43 - DNA, 26 bp, Artificial Sequence
  Description of Artificial Sequence Synthetic primer
ggacgacgaataaatgtcactccacc

SEQ ID NO: 44 - DNA, 24 bp, Artificial Sequence
  Description of Artificial Sequence Synthetic primer
cagcctggataccgctactcaaat

SEQ ID NO: 45 - DNA, 27 bp, Artificial Sequence
  Description of Artificial Sequence Synthetic primer
cagttactacgtatggagcagaactgt

SEQ ID NO: 46 - DNA, 24 bp, Artificial Sequence
  Description of Artificial Sequence Synthetic primer
cgatgcattcccagccactaagta

SEQ ID NO: 47 - DNA, 24 bp, Artificial Sequence
  Description of Artificial Sequence Synthetic primer
atgaatgcgctgacaaccgatgtg

SEQ ID NO: 48 - DNA, 24 bp, Artificial Sequence
  Description of Artificial Sequence Synthetic primer
aactgttggtgcgtttgggttacg

SEQ ID NO: 49 - DNA, 24 bp, Artificial Sequence
  Description of Artificial Sequence Synthetic primer
tgggaactgcatctgatactggca

SEQ ID NO: 50 - DNA, 24 bp, Artificial Sequence
  Description of Artificial Sequence Synthetic primer
tctacgcatttcaccgctacacct

SEQ ID NO: 51 - DNA, 22 bp, Artificial Sequence
  Description of Artificial Sequence Synthetic primer
gtatcgctgaaattagaagcgc

SEQ ID NO: 52 - DNA, 21 bp, Artificial Sequence
  Description of Artificial Sequence Synthetic primer
agttgaaacacccgtaatggc

SEQ ID NO: 53 - DNA, 21 bp, Artificial Sequence
  Description of Artificial Sequence Synthetic primer
tgttccaggtggtaggattcg

SEQ ID NO: 54 - DNA, 21 bp, Artificial Sequence
  Description of Artificial Sequence Synthetic primer
tcacgatgttgatcatctggg

SEQ ID NO: 55 - DNA, 18 bp, Artificial Sequence
  Description of Artificial Sequence Synthetic primer
cgttgtgcatggtggcat

SEQ ID NO: 56 - DNA, 22 bp, Artificial Sequence
  Description of Artificial Sequence Synthetic primer
tggccaaaccaactatgaactg

SEQ ID NO: 57 - DNA, 22 bp, Artificial Sequence
  Description of Artificial Sequence Synthetic primer
aggcgctgtttggtctcttaga

SEQ ID NO: 58 - DNA, 20 bp, Artificial Sequence
  Description of Artificial Sequence Synthetic primer
gaaccgaaatgatgggtgct

SEQ ID NO: 59 - DNA, 20 bp, Artificial Sequence
  Description of Artificial Sequence Synthetic primer
ttggagcgttaactggacct

SEQ ID NO: 60 - DNA, 24 bp, Artificial Sequence
  Description of Artificial Sequence Synthetic primer
atattcgctatatcttcttgcggc

SEQ ID NO: 61 - DNA, 19 bp, Artificial Sequence
  Description of Artificial Sequence Synthetic primer
aaactgggattggacgtgg

SEQ ID NO: 62 - DNA, 19 bp, Artificial Sequence
  Description of Artificial Sequence Synthetic primer
cccaaaacttctaggcccg

SEQ ID NO: 63 - DNA, 1318 bp, Escherichia coli
ttaattttgatgccagccaggttggtcattctcaaatacctcagcctcggggaatattttagtcaatgggataacatatttgaccagatcatgggtaaaaaagtcatccgcagtttcatctaccatccacgcagagttctcatctatgcgtggcacgagataatgaacattcaggcggactattttagatttttgctcaaaatttactatggcagcatagtttttttcccttcgtttatcggcaacatccttttgaatatctacatccaaagctatatgccctacgcctagaccataatccaaaagctggttatctatctctgctaatgtttttgttacatgtcgcgaacgtgcatcgaaagatttttcattgatacaacgccatgttatcaaagaagcaagtttaactttggttatgaaacgtgagtcgcgctcatctggtcgtcccatagcaacaacttggtaattttcatcttccagaggggagcctttgataagacgagcaagtttagtgttgactagtaatgagccgttttcagtgatatcttccctgataagatttaagttggccggtctaatcgttccttttccataatcatcattccattcataaaatacacctttaaagttttttaagtggctaatgatatagttttcaggcgcgtctttaacttcacataaatatgtgacatcagtccatacgttcatctttttggatttgatatgaagttcggcaagatgtgagcgtatatgatgctgtaatatttcctgctttacgtaaggacctttctgaagccttttgcattcgacaaagaaataatcgccatctccaaggcggcaacgaaactccggggtttttgctatccctttttgttctggaatgaactcaacttcataaccttctgatgcatagtttccagcaagaaccaactctaataaggctgtgtctggtaatactgttgtattcttaagcattctgattgcgcgatctctagcgccagtaattctatcgagcgatttagcacacactcctaattgtttaacccatggtattatatttgaagcattagtaacttcgtaagaccttctattatcgataagcgattttgcttgtgcgaaataaccagcaaccacgtcggaaccataccattctgggtcaaactgtttcccgaaattggcagctttttgggttgtagctatgtaaaattgctgtgcttttgctaagcgctggtaaaagtgttgtttgttctcttgagttgatgctaaccactcaagaccagcaataacgtcagaatctagcataacaccgtcattcatt

SEQ ID NO: 64 - DNA, 5612 bp, Escherichia coli
gaattccgggccatgatcctgatcaggaagataatatccccagtatccctgccggactgagcggacaaaggaggcaataccgtcattacgtgcgtgcagttttcctccgaaacgacgacgaacactgttccagacataatccaccaccggatttctctggttatgaaacatcgctgccatttttctgcccgacgcagccatcagcattgcgggaatatccactccccaggcatgtggcacaagaaaaatgaccttttcattgttctgtgccatcttctccacaatctccagaccattccagcgtatacgatgcgaaattttatcaggcccggacagtgctaattcagccataagcacaacggccatggaggctgttgcaaacatggcatcaacgatattctccttttctttatcactgtactccggaaaacaaagcgataaattaatcaaggcccgttgacgtgcgcttttccccagacgcccaaccagcatgcccaattttcccaacaatggatctctcagtgcgggggggaccattgcaaacacacagataataccgatacctgtccacgtcatccagtaacgggggaagcataatgtaaggttaaagcggggaataaactcagttctgatatttttcatatttaccggtatctgcctgcttatctcattatgataacagagtacagcagaaggtggtaccatccctggaggccaccggacagcagaggaaaacacatcctcctgccttcagaacggaaccgaagataagaaaatactatggctcaccatacggaagaggtcgaaatcagtgagcccattctttttatacggatttccaatccagcgggtggtttccctgaattggggattcaccagagaacgggtcggatcgtaaccatcgtatgtcagtcccgccaaatctgaccaggtgtggatcagttcagccagactgtatttgcggtcaacatactgcgaaaaatctcgcggatgtgcagcatgccacttttccgaggtccacagcaggaacggaacagtgtacatgggacgtgtggggttatcttcattccgtccctgcgttttatatggcggagtatcatatacttcttcaccatggtctgaaaaataaagcagaaagccgtccggcgcagttgctctgaagtcctttatcagactggccaccacatgatcgttaaacagattggcattatcgtaatcgttatacacttccagctctttcgcatttaatccagtgggaatatgccctgtaatgccatcaaaccgcccctgaccttccggataacggtatttatacttaatgtgcgtacccagcagatgcacgatgatcagtttctttggtgcagggtcattcagcacttcccggaacggcttcagcacgttagtgtcatattcacgcgcactttgtgttcgctgctgattcatgtagtactgcctgtccgtctggcgcgaaaatacagtgagcatggtgttacgggctgtgattgtctgctggttggtaatccagaatgttttatagcctgcctgcttcatcatgttcatcagcgacggctgcgtcagatacagatcaggattcttttcgttggcgaaagtaagggcctgttgcaatgcttcaatggtgtacggacgcgatgccaccacattattaaacacagtaagacccggatcggttttacgcagtgcatccagctccggcgtcgtttcacgtagatacccgtataagctcatgcgttcgcgctgtgtcgactcaccaatcaccaggaccagtgtgcgtggtctctcccctgattcatccctgagattacccagtggtggcagtgcgctattttcattcaggaaggttgtcagtgcattcagttgctgatggtactgataataactggatacaaactgccagggggcagcaggctccattcgggatgccagtttgcctagagtatcattcagcggctcctgtctgattaacgatttcagaacaaccggatgcagaagcagagcataaagcagcaggaaagagacaattctccgccatggcaaaggaatatatacaggacgcagacgtgtccacagaaaaacggacaccgcagtatataccagcgagataagcaacagtttaaggctgaaatactggctgaaatattcaccagcctctctggcattcgtttcaaacataacgaaaagaacactttgagagaattcatgaccatagagaaaataataacaaagtgctgccagcgacgtgccccagagaatgaaaccgacaacagctgcaattatttttatccgatcaggatagagaaacaccgggatcaaccacagacaactgaataacagtgagtcccgtattccattcgttccgctgtacccactgctgaagatgatgacctgcaggagagtggaaaaaaagccaaaataaaaaattgcccatcccagcgcgctccagctgaaagtaggcctgttctgtccggtatttaaatgcattgaccgtccccgtatttaaacaatgtgataaattactccgttaccggaaaaccgctgaacaaaattcgggctgaaaagaggatccgccgttatctgttgcatttccccttagcctgactagccagagacacaatgatctgtgccgttctgttaatatcaaaccggtactcaatatcttctctggcgctggctgccatcatccggaagcgttccggtcgggataaaaaatcgcgcagtgcgccggtccatgcagacacatcccccacgggtaacagcgtccctgtcacattcttctgaatgacatcagggatcccgcccgtctcactggcgataacgggcacgccggagactgacgcttcagccagtaccataccaaacgcttcattttccgaaggcatgaccaccacactggcaatccggtagaccggtaacgctgggaaaagggcacctgccattaacacatctccgctcattcccaggtgttctgtctgctgacgcagacttgcttcgtattcttcacgcccggcgcccaccacgagccagcgaaatgatttcccttccatcttcagctgatacaatacacgcagcataaattcatgtcctttttcgggacgtagcatccccacctgaacgataagcggaacattgtctgctgatgcagcccaggcgtggatatgcaggggtaacggtcgcatggcttcattatgcaatgcgggccagtcgaaacccggtggaataaccgttaccggtgtcctgacaccttccgccatcagatgcgccatcatggctgagctaggcacaacaatgaaatcacacagataattcagggaaaacgttctggtcttacgggtgatgtaggttttttgtctgacaatactgaagcggtgacagcatatcagacggctcagtcctgctatattactgtcatggccactatggcagatgaccagatcaggtttaaattccccgataatccgtcgaaatctgaggatggaaggaaggtgaaggctgttcctgaaaggaataaaagtgacatcatgccctctttttctggcttccggagcaattttacttttttctctgcaggcaagtaaaacggaatgtccctgcttttgaagagcagtcatctgggccagtgcctgcagctcctggcccccaatatctgacgaagattcagtgaataaaattttcatcattaattatctggtaatcttggccccttatgaagagccagtactgaagaaaaaagtggcgttgtataataaaaaaggcgtcgtttcagccagccggaatgttcccgctctttggtgctgatacgccctatgcgtaatgaaccattttcaggacagttcattcttctttcggttgtatataaataagagaaaccaagcttaccggcgagattaatataatcccgattataatatccttcaggccagcacagatgggaactacagaaccccagcttttccgtcagacattgttttcccaccagaatatcttctttcatgagccggcactgttcagcacgtgatacagacaacctgtcccagcgtttatgtgaatgggtgtgcacatgaaattcaaccagtccggaatcacgcatttcccggacttcagaccagcgaagcatgacctcatctgaacggttatcggcaatcaaccgttcacagtcgcggtgagaatattcctgctcctgcctgctacgtacgtttcccttaccaatcagccccgtaatgagaaaaatatgtgcatgaaggttatattccttcaggaccggccatgctctgagccagttatcaagataaccatcatcgaatgtgagcatgacactctttcgtggaagcgttcccccctgataaaaatattcaagctcagcagatgttaccgtccgccagttattatctgcgagccacttcatctgttcacaaaatgtttcaggtgagagtgtcacaagccccggacaacgactgacatgatgatacatcagtacgggaagatgccttgcattcaacataaaaaaataatcgtctgttaagcattctgaaaatatcctcgcgtaaacaacagtgaggatccatagatatcacacatggtgatattattgtgtaatcccggaatggtccggagattaccgacaacagggattttttaatatttttatcagttaatcaaccagaacttaaatttcccttaacgcatatgctctttttaatcagattttctgtttttcagaaaaaacagaataccgcaatttcagtaaacacagcgagagatatccgcctcagtaaaaaatcagaagattatcatcctgatttattgcataaaaccagtcgagtagcggaattttctgcatccggataccactccccctgaaaaatactcgcacaatatcgcgccgcatgcactgatggaccgggccatcgtgagaatctcttgcatcgtagatattaaccacaccagtgccccccagaacaacagtaatacctgtagcagacctgccatagaggttaaataaccattcatatccctcttctacccattcatcccgaagtttcagccagcgacgaatggactgtacctcctgtttcagccgaggggctgagttgacaaaccgtttagagccgccggatattcaggttaccggcctgcaaatcaatatcagacagttttatacctaataactctgatgcttggacaccatgaagataccccataaggagcaggcagtgattacgttccgggtgtggcactttgaagacagcactgtgcaacttatgaatctcacccgggttcagatatttttgttgtccattacctgtccttatttattgaaagtcgatattagtttaaaaagctgctaatcatgacaccattacagaagtaaaatcaatttattttaaatacataaaattattgttcatttatttttttgcaaacattcatgaactaaaaacaatggataaaaccaataatattgcataataatacacctcccttataaataatggagaagaaaatgaaaaggcggtatatcacctatgctgaacctgtttaggatgctgtgtaaatgccttttctccgaagtgaccgtccaagcggtcaccgaattc

SEQ ID NO: 65 - DNA, 949 bp, Escherichia coli
aggcaagtaaaacggaatgtccctgcttttgaagagcagtcatctgggccagtgcctgcagctcctggcccccaatatctgacgaagattcagtgaataaaattttcatcattaattatctggtaatcttggccccttatgaagagccagtactgaagaaaaaagtggcgttgtataataaaaaaggcgtcgtttcagccagccggaatgttcccgctctttggtgctgatacgccctatgcgtaatgaaccattttcaggacagttcattcttctttcggttgtatataaataagagaaaccaagcttaccggcgagattaatataatcccgattataatatccttcaggccagcacagatgggaactacagaaccccagcttttccgtcagacattgttttcccaccagaatatcttctttcatgagccggcactgttcagcacgtgatacagacaacctgtcccagcgtttatgtgaatgggtgtgcacatgaaattcaaccagtccggaatcacgcatttcccggacttcagaccagcgaagcatgacctcatctgaacggttatcggcaatcaaccgttcacagtcgcggtgagaatattcctgctcctgcctgctacgtacgtttcccttaccaatcagccccgtaatgagaaaaatatgtgcatgaaggttatattccttcaggaccggccatgctctgagccagttatcaagataaccatcatcgaatgtgagcatgacactctttcgtggaagcgttcccccctgataaaaatattcaagctcagcagatgttaccgtccgccagttattatctgcgagccacttcatctgttcacaaaatgtttcaggtgagagtgtcacaagccccggacaacgactgacatgatgatacatcagtacgggaagatgccttgcattcaacataaaaaaataatcgtct

SEQ ID NO: 66 - DNA, 1150 bp, Escherichia coli
gctctttcgcatttaatccagtgggaatatgccctgtaatgccatcaaaccgcccctgaccttccggataacggtatttatacttaatgtgcgtacccagcagatgcacgatgatcagtttctttggtgcagggtcattcagcacttcccggaacggcttcagcacgttagtgtcatattcacgcgcactttgtgttcgctgctgattcatgtagtactgcctgtccgtctggcgcgaaaatacagtgagcatggtgttacgggctgtgattgtctgctggttggtaatccagaatgttttatagcctgcctgcttcatcatgttcatcagcgacggctgcgtcagatacagatcaggattcttttcgttggcgaaagtaagggcctgttgcaatgcttcaatggtgtacggacgcgatgccaccacattattaaacacagtaagacccggatcggttttacgcagtgcatccagctccggcgtcgtttcacgtagatacccgtataagctcatgcgttcgcgctgtgtcgactcaccaatcaccaggaccagtgtgcgtggtctctcccctgattcatccctgagattacccagtggtggcagtgcgctattttcattcaggaaggttgtcagtgcattcagttgctgatggtactgataataactggatacaaactgccagggggcagcaggctccattcgggatgccagtttgcctagagtatcattcagcggctcctgtctgattaacgatttcagaacaaccggatgcagaagcagagcataaagcagcaggaaagagacaattctccgccatggcaaaggaatatatacaggacgcagacgtgtccacagaaaaacggacaccgcagtatataccagcgagataagcaacagtttaaggctgaaatactggctgaaatattcaccagcctctctggcattcgtttcaaacataacgaaaagaacactttgagagaattcatgaccatagagaaaataataacaaagtgctgccagcgacgtgccccagagaatgaaaccgacaacagctgcaattatttttatccgatcaggatagagaaacaccgggatcaaccacagacaactgaataacagtgagtcccgtattccattcgttccgctgta

SEQ ID NO: 67 - DNA, 1269 bp, Escherichia coli
tcaaaaggaaactatattcagaagtttgagggttaactgttatgttgtactgcttcatttttatatatattgtaaattactttatatggtataaatgtagttttaaaaaacatatcgatagacagttaaatataagaggatgaaaatgaaatatataccagtttaccaaccgtcattgacaggaaaagaaaaagaatatgtaaatgaatgtctggactcaacgtggatttcatcaaaaggaaactatattcagaagtttgaaaataaatttgcggaacaaaaccatgtgcaatatgcaactactgtaagtaatggaacggttgctcttcatttagctttgttagcgttaggtatatcggaaggagatgaagttattgttccaacactgacatatatagcatcagttaatgctataaaatacacaggagccacccccattttcgttgattcagataatgaaacttggcaaatgtctgttagtgacatagaacaaaaaatcactaataaaactaaagctattatgtgtgtccatttatacggacatccatgtgatatggaacaaattgtagaactggccaaaagtagaaatttgtttgtaattgaagattgcgctgaagcctttggttctaaatataaaggtaaatatgtgggaacatttggagatatttctacttttagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactaggaccgcagaggaaagagaggaattaaggaatcaccttgcagataaactcatcgaaacaaggccagttttttaccctgtccacacgatgccaatgtactcggaaaaatatcaaaagcaccctatagctgaggatcttggttggcgtggaattaatttacctagtttccccagcctatcgaatgagcaagttatttatatttgtgaatctattaacgaattttatagtgataaatagcctaaaatattgtaaaggtcattcatgaa

SEQ ID NO: 68 - DNA, 1392 bp, Escherichia coli
atgataatgaataaaatcaaaaaaatacttaaattttgcactttaaaaaaatatgatacatcaagtgctttaggtagagaacaggaaaggtacaggattatatccttgtctgttatttcaagtttgattagtaaaatactctcactactttctcttatattaactgtaagtttaactttaccttatttaggacaagagagatttggtgtatggatgactattaccagtcttggtgctgctctgacatttttggacttaggtataggaaatgcattaacaaacaggatcgcacattcatttgcgtgtggcaaaaatttaaagatgagtcggcaaattagtggtgggctcactttgctggctggattatcgtttgtcataactgcaatatgctatattacttctggcatgattgattggcaactagtaataaaaggtataaacgagaatgtgtatgcagagttacaacactcaattaaagtctttgtaatcatatttggacttggaatttattcaaatggtgtgcaaaaagtttatatgggaatacaaaaagcctatataagtaatattgttaatgccatatttatattgttatctattattactctagtaatatcgtcgaaactacatgcgggactaccagttttaattgtcagcactcttggtattcaatacatatcgggaatctatttaacaattaatcttattataaagcgattaataaagtttacaaaagttaacatacatgctaaaagagaagctccatatttgatattaaacggttttttcttttttattttacagttaggcactctggcaacatggagtggtgataactttataatatctataacattgggtgttacttatgttgctgtttttagcattacacagagattatttcaaatatctacggtccctcttacgatttataacatcccgttatgggctgcttatgcagatgctcatgcacgcaatgatactcaatttataaaaaagacgctcagaacatcattgaaaatagtgggtatttcatcattcttattggccttcatattagtagtgttcggtagtgaagtcgttaatatttggacagaaggaaagattcaggtacctcgaacattcataatagcttatgctttatggtctgttattgatgctttttcgaatacatttgcaagctttttaaatggtttgaacatagttaaacaacaaatgcttgctgttgtaacattgatattgatcgcaattccagcaaaatacatcatagttagccattttgggttaactgttatgttgtactgcttcatttttatatatattgtaaattactttatatggtataaatgtagttttaaaaaacatatcgatagacagttaaatataagaggatga

SEQ ID NO: 69 - DNA, 1185 bp, Escherichia coli
gtgaagtcagcggctaagttgatttttttattcctatttacactttatagtctccagttgtatggggttatcatagatgatcgtataacaaattttgatacaaaggtattaactagtattataattatatttcagattttttttgttttattattttatctaacgattataaatgaaagaaaacagcagaaaaaatttatcgtgaactgggagctaaagttaatactcgttttcctttttgtgactatagaaattgctgctgtagttttatttcttaaagaaggtattcctatatttgatgatgatccagggggggctaaacttagaatagctgaaggtaatggactttacattagatatattaagtattttggtaatatagttgtgtttgcattaattattctttatgatgagcataaattcaaacagaggaccatcatatttgtatattttacaacgattgctttatttggttatcgttctgaattggtgttgctcattcttcaatatatattgattaccaatatcctgtcaaaggataaccgtaatcctaaaataaaaagaataatagggtattttttattggtaggggttgtatgctcgttgttttatctaagtttaggacaagacggagaacaaaatgactcatataataatatgttaaggataattaataggttaacaatagagcaagttgaaagtgttccatatgttgtttctgaatctattaagaacgatttctttccgacaccagagttagaaaaggaattaaaagcaataataaatagaatacagggaataaagcatcaagacttattttatggagaacggttacataaacaagtatttggagacatgggagcaaattttttatcagttactacgtatggagcagaactgttagttttttttggttttctctgtgtattcattatccctttagggatatatatacctttttatcttttaaagagaatgaaaaaaacccatagctcgataaattgcgcattctattcatatatcattatgattttattgcaatacttagtggctgggaatgcatcggccttcttttttggtccttttctctccgtattgataatgtgtactcctctgatcttattgcatgatacgttaaagagattatcacgaaatgaaaatatcagttataactgtgacttataa

SEQ ID NO: 70 - DNA, 2634 bp, Escherichia coli
tgcgacgctgacgcgtcttatcatgcccggaagtctgcgcccgaatcgtaggccggataaggcgtttacgccgcatccggcagtcgtgcaccgacgcctgatgcgacgcgggcgcgtcatatcacgccaaaaccgtaggccgcctccgccatgttaaatgttaactggcattggcaatttactcttcccggcctttactcatacttttttggtcttcatccggatagtgtttttttagatattccaggacgtttttattgaccttgtgttgcgtatacacccaccctttccagtaatcaggctggtccaggtaaacttctggcggaatggtgaaatcagaaagcgttaaccattcggctaacagatcggggtttcgtttctgtatcaactgcaacagcataatcagcgacatggcagaggcaggagccgtactatcgccgcttaaatacttccacactgtcgaccggttcgaacgaaaaaggatcggtagcaatgcccggtccagattcatttcattaaaaatcttctcaaattcattcatttaaattttcctgcctggcgtaaacctcttaaaaattgagatttatcaaagaaacgcattttagcacacatcaggaaccgcttcacgtttagtccagaaacagaatttatttcgcttatcaaaacaagtctttactcttttttacattgaaagagcacgaaatgatttccttttttatttatataagaaaccatttttgtttcttattgatggtgtttacgcttacaacagacaaaaatgcgctttacatcacacaaatggcggcgtagatttcgattaaattgcaacgcagtttatttcttaaaacaatattatttgtttcttatagaaacattaatacgacttattttgaacaagagaaaatgaatgaaaactgtaaacgtagctttactggcactcataatttcagcaacatccagccctgttgttttagctggtgataccattgaagcggcggcaacagagctttcagccattaactctggcatgtcgcaatcggagattgagcagaagattacccgctttttagaacgcacagacaacagccccgctgcgtatacctatttgactgaacatcactacatcccttctgaaacacctgataccactcagactcccactgtccagacagatcctgacgcaggacaaaaaaccgttgccgctacaggtgatgtacagacaactgcccgttatcagagcatgatcaacgcccgacagtctgcggtaactgacgcccagcaaacgcaaattacagagcaacaggcgcagatcgtagccacacaaaaaacgctcgccgcgactggagatacgcaaaataccgcgcattatcaggaaatgattaatgccagactggcggctcaaaatgaggctaatcagcgcaccgccactgaacaagggcagaaaatgaatgcgctgacaaccgatgtggcagtacaacagcaaaatgaaaggactcaatacgataaacaaatgcaaagtctggcgcaggagtctgcccaggcacatgaacaaattgacagcctgtcacaagacgtaacccaaacgcaccaacagttaaccaacacccaaaaacgggttgcagataacagccagcaaattaacacgctcaataaccatttcagttcgctaaaaaacgaagttgatgacaatcgtaaagaagccaatgcgggaactgcatctgccatcgctatcgcctcacaaccacaggttaaaaccggtgacgtgatgatggtgtcagcgggagcgggaaccttcaacggtgaatctgcggtgtctgtcggaacatcatttaatgccggaacgcatacggtacttaaagccggtatttctgcggatacacaatctgatttcggcgcaggtgtcggcgtgggatattcgttctaatatttcaatcctcaatataaataagagcaaggaagcttgccgggttcacctcttcattaatttgtacattatttaaggttaacaatgatgaatagctccattaaatcgttttccctgctggcggttatattactggctggctgtagttcacccacttcccgcatcgcagattgccaggcgcagggcgtcagtcatgacacctgttacctcgcagaacagcagcgtcaggcggctattttaagtgcatccgaggcacaggcatttaaaaatgcagaagccgcacaacacgcccaggcggcaaagaaagccatttataaaggatttggcatgacctttagaatgagcagtaaaaactttgcttatctcaatgattcattatgtgcaattgatgaagacaataaagatgccactgtttatcagtcaggtctatataacgtcattgtttatcatcacacaggaaaagtcgccttaatgaaagaaggccagtttgtgggttatttaaaatgaaggagcaaaggaaaatacccctgacgcatattatgattatcggtgcgtttatttttgccttcttgcaagtagtattattagcctccctggttcacgctgtgaatgttaacaacgaaatccaggaaggcttatttcagtcggggcgcattatggtagaaagtttgcagcatattctttcggtgcaaacggggattcactgattt

SEQ ID NO: 71 - DNA, 279 bp, Escherichia coli
atgtctttatatataaaactcatcctctcaattgtcagggaaattagcgtgaatactatctgttctttaattgttgtggttgcactgtctttattatcattcagtagcgtcgccaaaacgattactgccgtcggttcaaccattaacagcactgaaaaagaaatttctttacaggcagaaaaacaagggaaatcatataaaattctgggcgcgttttttaagaacagagtttatatgatagcaaagttaacaccagtcagtaaaaatgatgcttcataa

SEQ ID NO: 72 - DNA, 357 bp, Escherichia coli
ttatttcttctcgcagtttcgcatcttatagaagaatcctgtatttccatcttccacgatgaagcgatcgttaaaagttggacgacgaataaatgtcactccacctattgttgctttttgttttataccatcatcaacattttccagatctggagagtacaattgatctccatttggcatttgaacgatgaaattgtttccattatcgagaactgttgcggattttgacagaatttgagtagcggtatccaggctggaaatcctaatgtcacaaacatatttgcctggccccagatcttcttctgcaaatacaggtaaggaaaataatgcaaatgcgaggatgagattcttattcat

SEQ ID NO: 73 - DNA, 1489 bp, Escherichia coli
ctgcagtccggagatgaaagcaccactgtgtgtaccccatcagcgtggtcccgcaggccatgatttttgtcacagactcaatgactaccggacgcactgaaccttccggttgtttctccagccagttaagccagcggtttccctgctgaaaaatgtcggcaaaacggggaagcatcagaagggcgggggaactccgtccggccagtgaaccgtgccacactccgggcagtacatgccgccggcgctgataccggcaagaatggtcgcaaactcccgctccgtgcagcgggctatttcaggatacccttcgtcatcaacacgtacaaaccagaagaccagctttttgtttctgacatccacaaagaagggaatattcaggtctgcgcagcactcaacggcatcgtcagttgcggcttggaaccccttagtattttttgtctgtagtatctatcccagcaataggtatatcctgttgcatcaataaagttgacttttgtatacaacatgcgaatttcccttaatccggagctattcgtatgataaaaaaaactcttcctgttctgattcttctggcgctatcggggagcttttctaccgctgtagccgctgataaaaaagagactcaaaatttctactatccagaaacactggatttaactcctctgagattacacagccctgaatcaaatccctggggggctgattttgattatgccaccagatttcaacagctggatatggaggctctgaaaaaagatatcaaagatttgctgacaacttcccaggattggtgccctgcggattatggtcattatggtcctttctttattcgtatggcttggcacggtgccggaacatacaggacatatgatggccggggaggcgccagtggtggtcagcaacgttttgaaccgctgaacagctggccggataacgttaatctggataaagcccgtcgattgctgtggccagtcaagaaaaaatacggctccagtatttcctggggagacctgatggtcctgactggtaatgttgcccttgaatccatgggatttaaaacgctgggatttgctggcggaagagaagatgactgggagtcggacctggtatactgggggcctgacaacaagcctcttgcagataaccgggataaaaacgggaaacttcagaaacctcttgccgccacgcagatgggacttatttatgtcaatcctgaaggccccggtggaaaaccagatcctctggcttccgcgaaagatatcagggaagctttttcacgtatggccatggatgatgaggagactgtggccctgatcgcgggagggcatacatttggtaaagcacatggtgcagcgtctcctgaaaaatgtattggcgcagggcctgatggtgcacctgtggaggagcagggactgggatggaaaaataaatgtggtacaggaaacggcaaatataccatcaccagtggcctggaaggagcctggtcgac

SEQ ID NO: 74 - DNA, 23 bp, Artificial Sequence
  Description of Artificial Sequence Synthetic primer
agaagggaatattcaggtctgcg

SEQ ID NO: 75 - DNA, 27 bp, Artificial Sequence
  Description of Artificial Sequence Synthetic primer
cctattgctgggatagatactacagac

SEQ ID NO: 76 - DNA, 822 bp, Escherichia coli
ttaattatctggtaatcttggccccttatgaagagccagtactgaagaaaaaagtggcgttgtataataaaaaaggcgtcgtttcagccagccggaatgttcccgctctttggtgctgatacgccctatgcgtaatgaaccattttcaggacagttcattcttctttcggttgtatataaataagagaaaccaagcttaccggcgagattaatataatcccgattataatatccttcaggccagcacagatgggaactacagaaccccagcttttccgtcagacattgttttcccaccagaatatcttctttcatgagccggcactgttcagcacgtgatacagacaacctgtcccagcgtttatgtgaatgggtgtgcacatgaaattcaaccagtccggaatcacgcatttcccggacttcagaccagcgaagcatgacctcatctgaacggttatcggcaatcaaccgttcacagtcgcggtgagaatattcctgctcctgcctgctacgtacgtttcccttaccaatcagccccgtaatgagaaaaatatgtgcatgaaggttatattccttcaggaccggccatgctctgagccagttatcaagataaccatcatcgaatgtgagcatgacactctttcgtggaagcgttcccccctgataaaaatattcaagctcagcagatgttaccgtccgccagttattatctgcgagccacttcatctgttcacaaaatgtttcaggtgagagtgtcacaagccccggacaacgactgacatgatgatacatcagtacgggaagatgccttgcattcaacat

SEQ ID NO: 77 - protein, 273 aa, Escherichia coli
MLNARHLPVLMYHHVSRCPGLVTLSPETFCEQMKWLADNNWRTVTSAELEYFYQGGTLPRKSVMLTFDDGYLDNWLRAWPVLKEYNLHAHIFLITGLIGKGNVRSRQEQEYSHRDCERLIADNRSDEVMLRWSEVREMRDSGLVEFHVHTHSHKRWDRLSVSRAEQCRLMKEDILVGKQCLTEKLGFCSSHLCWPEGYYNRDYINLAGKLGFSYLYTTERRMNCPENGSLRIGRISTKEREHSGWLKRRLFYYTTPLFSSVLALHKGPRLPDN

SEQ ID NO: 78 - DNA, 1107 bp, Escherichia coli
ttatctgttgcatttccccttagcctgactagccagagacacaatgatctgtgccgttctgttaatatcaaaccggtactcaatatcttctctggcgctggctgccatcatccggaagcgttccggtcgggataaaaaatcgcgcagtgcgccggtccatgcagacacatcccccacgggtaacagcgtccctgtcacattcttctgaatgacatcagggatcccgcccgtctcactggcgataacgggcacgccggagactgacgcttcagccagtaccataccaaacgcttcattttccgaaggcatgaccaccacactggcaatccggtagaccggtaacgctgggaaaagggcacctgccattaacacatctccgctcattcccaggtgttctgtctgctgacgcagacttgcttcgtattcttcacgcccggcgcccaccacgagccagcgaaatgatttcccttccatcttcagctgatacaatacacgcagcataaattcatgtcctttttcgggacgtagcatccccacctgaacgataagcggaacattgtctgctgatgcagcccaggcgtggatatgcaggggtaacggtcgcatggcttcattatgcaatgcgggccagtcgaaacccggtggaataaccgttaccggtgtcctgacaccttccgccatcagatgcgccatcatggctgagctaggcacaacaatgaaatcacacagataattcagggaaaacgttctggtcttacgggtgatgtaggttttttgtctgacaatactgaagcggtgacagcatatcagacggctcagtcctgctatattactgtcatggccactatggcagatgaccagatcaggtttaaattccccgataatccgtcgaaatctgaggatggaaggaaggtgaaggctgttcctgaaaggaataaaagtgacatcatgccctctttttctggcttccggagcaattttacttttttctctgcaggcaagtaaaacggaatgtccctgcttttgaagagcagtcatctgggccagtgcctgcagctcctggcccccaatatctgacgaagattcagtgaataaaattttcatcat

SEQ ID NO: 79 - protein, 368 aa, Escherichia coli
MMKILFTESSSDIGGQELQALAQMTALQKQGHSVLLACREKSKIAPEARKRGHDVTFIPFRNSLHLPSILRFRRIIGEFKPDLVICHSGHDSNIAGLSRLICCHRFSIVRQKTYITRKTRTFSLNYLCDFIVVPSSAMMAHLMAEGVRTPVTVIPPGFDWPALHNEAMRPLPLHIHAWAASADNVPLIVQVGMLRPEKGHEFMLRVLYQLKMEGKSFRWLVVGAGREEYEASLRQQTEHLGMSGDVLMAGALFPALPVYRIASVVVMPSENEAFGMVLAEASVSGVPVIASETGGIPDVIQKNVTGTLLPVGDVSAWTGALRDFLSRPERFRMMAASAREDIEYRFDINRTAQIIVSLASQAKGKCNR

SEQ ID NO: 80 - DNA, 1749 bp, Escherichia coli
tcagaacggaaccgaagataagaaaatactatggctcaccatacggaagaggtcgaaatcagtgagcccattctttttatacggatttccaatccagcgggtggtttccctgaattggggattcaccagagaacgggtcggatcgtaaccatcgtatgtcagtcccgccaaatctgaccaggtgtggatcagttcagccagactgtatttgcggtcaacatactgcgaaaaatctcgcggatgtgcagcatgccacttttccgaggtccacagcaggaacggaacagtgtacatgggacgtgtggggttatcttcattccgtccctgcgttttatatggcggagtatcatatacttcttcaccatggtctgaaaaataaagcagaaagccgtccggcgcagttgctctgaagtcctttatcagactggccaccacatgatcgttaaacagattggcattatcgtaatcgttatacacttccagctctttcgcatttaatccagtgggaatatgccctgtaatgccatcaaaccgcccctgaccttccggataacggtatttatacttaatgtgcgtacccagcagatgcacgatgatcagtttctttggtgcagggtcattcagcacttcccggaacggcttcagcacgttagtgtcatattcacgcgcactttgtgttcgctgctgattcatgtagtactgcctgtccgtctggcgcgaaaatacagtgagcatggtgttacgggctgtgattgtctgctggttggtaatccagaatgttttatagcctgcctgcttcatcatgttcatcagcgacggctgcgtcagatacagatcaggattcttttcgttggcgaaagtaagggcctgttgcaatgcttcaatggtgtacggacgcgatgccaccacattattaaacacagtaagacccggatcggttttacgcagtgcatccagctccggcgtcgtttcacgtagatacccgtataagctcatgcgttcgcgctgtgtcgactcaccaatcaccaggaccagtgtgcgtggtctctcccctgattcatccctgagattacccagtggtggcagtgcgctattttcattcaggaaggttgtcagtgcattcagttgctgatggtactgataataactggatacaaactgccagggggcagcaggctccattcgggatgccagtttgcctagagtatcattcagcggctcctgtctgattaacgatttcagaacaaccggatgcagaagcagagcataaagcagcaggaaagagacaattctccgccatggcaaaggaatatatacaggacgcagacgtgtccacagaaaaacggacaccgcagtatataccagcgagataagcaacagtttaaggctgaaatactggctgaaatattcaccagcctctctggcattcgtttcaaacataacgaaaagaacactttgagagaattcatgaccatagagaaaataataacaaagtgctgccagcgacgtgccccagagaatgaaaccgacaacagctgcaattatttttatccgatcaggatagagaaacaccgggatcaaccacagacaactgaataacagtgagtcccgtattccattcgttccgctgtacccactgctgaagatgatgacctgcaggagagtggaaaaaaagccaaaataaaaaattgcccatcccagcgcgctccagctgaaagtaggcctgttctgtccggtatttaaatgcat

SEQ ID NO: 81 - protein, 582 aa, Escherichia coli
MHLNTGQNRPTFSWSALGWAIFYFGFFSTLLQVIIFSSGYSGTNGIRDSLLFSCLWLIPVFLYPDRIKIIAAVVGFILWGTSLAALCYYFLYGHEFSQSVLFVMFETNAREAGEYFSQYFSLKLLLISLVYTAVSVFLWTRLRPVYIPLPWRRIVSFLLLYALLLHPVVLKSLIRQEPLNDTLGKLASRMEPAAPWQFVSSYYQYHQQLNALTTFLNENSALPPLGNLRDESGERPRTLVLVIGESTQRERMSLYGYLRETTPELDALRKTDPGLTVFNNVVASRPYTIEALQQALTFANEKNPDLYLTQPSLMNMMKQAGYKTFWITNQQTITARNTMLTVFSRQTDRQYYMNQQRTQSAREYDTNVLKPFREVLNDPAPKKLIIVHLLGTHIKYKYRYPEGQGRFDGITGHIPTGLNAKELEVYNDYDNANLFNDHVVASLIKDFRATAPDGFLLYFSDHGEEVYDTPPYKTQGRNEDNPTRPMYTVPFLLWTSEKWHAAHPRDFSQYVDRKYSLAELIHTWSDLAGLTYDGYDPTRSLVNPQFRETTRWIGNPYKKNGLTDFDLFRMVSHSIFLSSVPF

SEQ ID NO: 82 - DNA, 705 bp, Escherichia coli
gaattccgggccatgatcctgatcaggaagataatatccccagtatccctgccggactgagcggacaaaggaggcaataccgtcattacgtgcgtgcagttttcctccgaaacgacgacgaacactgttccagacataatccaccaccggatttctctggttatgaaacatcgctgccatttttctgcccgacgcagccatcagcattgcgggaatatccactccccaggcatgtggcacaagaaaaatgaccttttcattgttctgtgccatcttctccacaatctccagaccattccagcgtatacgatgcgaaattttatcaggcccggacagtgctaattcagccataagcacaacggccatggaggctgttgcaaacatggcatcaacgatattctccttttctttatcactgtactccggaaaacaaagcgataaattaatcaaggcccgttgacgtgcgcttttccccagacgcccaaccagcatgcccaattttcccaacaatggatctctcagtgcgggggggaccattgcaaacacacagataataccgatacctgtccacgtcatccagtaacgggggaagcataatgtaaggttaaagcggggaataaactcagttctgatatttttcatatttaccggtatctgcctgcttatctcattatgataacagagtacagcagaaggtggtaccat

SEQ ID NO: 83 - protein, 235 aa, Escherichia coli
MVPPSAVLCYHNEISRQIPVNMKNIRTEFIPRFNLTLCFPRYWMTWTGIGIICVFAMVPPALRDPLLGKLGMLVGRLGKSARQRALINLSLCFPEYSDKEKENIVDAMFATASMAVVLMAELALSGPDKISHRIRWNGLEIVEKMAQNNEKVIFLVPHAWGVDIPAMLMAASGRKMAAMFHNQRNPVVDYVWNSVRRRFGGKLHARNDGIASFVRSVRQGYWGYYLPDQDHGPEF

SEQ ID NO: 84 - DNA, 1263 bp, Escherichia coli
atgttgaaaaaaaaacttcaaaaaataaaggaatatcattcagtattggagttggcaataattcagggtgcgaatgccatatttcctgtgttggtattcccattttttcttattaccttaggggaaaacatcttttcaagtattgctgttggtgaagtactagcactatatgtgcttatattttcgctatacagttttgatattataagtgtgcagaaggtaatttcaagtgtgacaaaagatgaaatatttaaagtttacattctgacactaatctgtaggttgtgtttatttgttatttcaggaatatgtcttttatttataacgtatttaattaataaaacattaagtgtatacttgggattgtttttattgtacccagtagggatgatattgcaatctaattatttttttcaggctacgaataacaataggccattggctgtttttgtactaattgctcgtggtatgtcattatgtcttatttatttttataatggaccagcaggctatttaacaagttattattatgtcatttgtgtgtctggttcgtattttttatctggcgtgctatcgcttatatatatatattatcaaaataagactaataaagctaaaattcaatgggcggaaattttagaatatatatgcacaggttatcatctgtttattgctaatatatttgttattctatacagaaatagtaatattattattcttggcactcttgcttcgcctgttgcaacgtctctgtacgcgacggcagagaaaattattaaatgtattcagtctatagcaaccccgttaaatcaatactatttcacgaggttgataaagcaacatgaattgaaattagaaccatacaaagttggagaatataaaagcctgctatatgcaagcacaaatattcagctaaagttcatggttttcattgtcctgagtttagggggggtgggtactatattgggatataaggttcaaagtatcgctgaaattagaagcgcgttcatccctttatcaataatgtcttttgcaatatttatggggatatacaattttatgtttggttcggttggattgtccataagagggtataaaaaagaattttcttatatagtggccattacgggtgtttcaactattattttatcattatgcctgagttatttctttgctgaaataggcgctgcaattgcttatgtatttgctgagtttatcttacttattctcatacttagaatttataaagtgaaacgattataa

SEQ ID NO: 85 - protein, 420 aa, Escherichia coli
MLKKKLQKIKEYHSVLELAIIQGANAIFPVLVFPFFLITLGENIFSSIAVGEVLALYVLIFSLYSFDIISVQKVISSVTKDEIFKVYILTLICRLCLFVISGICLLFITYLINKTLSVYLGLFLLYPVGMILQSNYFFQATNNNRPLAVFVLIARGMSLCLIYFYNGPAGYLTSYYYVICVSGSYFLSGVLSLIYIYYQNKTNKAKIQWAEILEYICTGYHLFIANIFVILYRNSNIIILGTLASPVATSLYATAEKIIKCIQSIATPLNQYYFTRLIKQHELKLEPYKVGEYKSLLYASTNIQLKFMVFIVLSLGGVGTILGYKVQSIAEIRSAFIPLSIMSFAIFMGIYNFMFGSVGLSIRGYKKEFSYIVAITGVSTIILSLCLSYFFAEIGAAIAYVFAEFILLILILRIYKVKRL

SEQ ID NO: 86 - DNA, 1260 bp, Escherichia coli
atgtctaattttttattatcagccataaaaaaaaatgtggcggatgaagataaaagaaaagtctttgtaaacttcttttatcttagcctcgagagaatcactcaacttgttatcattattatcattaacagattattgataaatcactatgattttggtgatttcagcagttggcaatattctctcgtcattttagccatatttatgacaggcacatggatctgtggagccgagatggtaattcctaaattactggatgcaccatctaacataaataccacaatgagcaacgtgattatattacgtctggctgcagggactttcgttgcgttgtgcatggtggcatggggattttttgctgcaagtgggctgtccagacagttcatagttggtttggccatatctgtcgcattacgagaaactttcatcgttggtttaacatggtatcaaagccaggctcgtctcaaattaccttgtctggtacttatgttagctgcaataattaaactcattgttatttatattggtttaaggaatcagttgccaataaattatctttgggtagcatgggtcattgaatcacttttaccttgtggctttatcttttattttttcagaaaggctaccgggtttagatttgtgaaggttaattctgaaattttctcatatcttaagattggtgttgccatatggtgttgtttgatcatgcaacaggtcactatgaagtttgatcgtgtttatctggaaggtaaaatttccggagaaatgtattcaaactatgcagcagcacttcaattggtcgataactggtatgcaatatgtattctgtttgtccaggcaattgcaccaatttttatatttaaattcattgatataataaatattaaaagaaaattgcctttttgtgttttcgctacacttgcagtaacctgcacgggcgcattatttacaacagttcttgcagacatgatcattcatattctttatggtgaaaagcttgttaatgcctatagttatttgcgaacgtttgtctggttaaccccaattttagctatagatcaactgctcagcatggttataattagaatgaatcagctaaataaattagctattaagtggttgatagctttctgcctagtagtggctgttgtgcctgctatttaccattatatgggtattagtaacatcgtcatcggtttagcagtagtttactccttcaatataatttattctacgaggtgcattggtgcagtacaatga

SEQ ID NO: 87 - protein, 419 aa, Escherichia coli
MSNFLLSAIKKNVADEDKRKVFVNFFYLSLERITQLVIIIIINRLLINHYDFGDFSSWQYSLVILAIFMTGTWICGAEMVIPKLLDAPSNINTTMSNVIILRLAAGTFVALCMVAWGFFAASGLSRQFIVGLAISVALRETFIVGLTWYQSQARLKLPCLVLMLAAIIKLIVIYIGLRNQLPINYLWVAWVIESLLPCGFIFYFFRKATGFRFVKVNSEIFSYLKIGVAIWCCLIMQQVTMKFDRVYLEGKISGEMYSNYAAALQLVDNWYAICILFVQAIAPIFIFKFIDIINIKRKLPFCVFATLAVTCTGALFTTVLADMIIHILYGEKLVNAYSYLRTFVWLTPILAIDQLLSMVIIRMNQLNKLAIKWLIAFCLVVAVVPAIYHYMGISNIVIGLAVVYSFNIIYSTRCIGAVQ

SEQ ID NO: 88 - DNA, 1266 bp, Escherichia coli
atggaaaagctaaatttcgcaaaagttacgatactcagtggtgtagtaacatgctttagactaatttgtggccttcttatttcaaaagttgttgcaatttatactggtccaattggaatagcaaatttgggacaattgcaaaattttgtggcctttataaatggattcatttcatcacaagtttcacaaggagttaatagatattcagcagaaaatcaaggtgattacgaaaatgcatgtttttattggcgtgcatcaattaaattatccttcatagcctgttgttttattataagtacgggggtgcttttttccagtaaaatatcagaatacttattttatcaaagtgatttgtattggttaattataattgctctatgcgctcttcctcttaatatagtaaacaatatatttttaggggttttaaatggactaaatgaatataatagatttttccttgcgaatgttttagccatatcctcatcgttgttatctatggtgggcttagtttatttttttggcctcaaaggcgcattagtgtctgcatcattaaataatgcggttgcaggtgtctggcttattactattataattaaaagaccatggtttaaatttaaatactgggtgggacatactcctcggcataacatcactcagatgaaaaattacttttatatgggggtgattggagcgttaactggacctatttcgatgatagttgttcgaactattctcactaataatttttctttagaggatgccggatattggcaagctgtaaatagaatttctgaggcttatctggctgttcttactacggcattgacagtttattattttccaaaaactgcagccgcaagaagatatagcgaatatataactcttttaaagaccggggcttgtattgttgtaccgctcgcgttgagcatggctttaaccatctacggcttgaaagattttataattagtatcttgttcacggctgactttatacgtgctcgggagctttttttatttcaaaatatcggggattttctgcggatttgtagttggctgtttgccactatattgttagcaaagggatattttaaaataaatgctatattggaggttatgttttcaataatttttccagtattggtaaagtttttgataattaattttggactaaatgccgttagtattgcctacagcatcacttatgcattgtattttatagtggtgtgtattatttattgttggcatctgacaaaaaaggccctataa

SEQ ID NO: 89 - protein, 420 aa, Escherichia coli
MEKLNFAKVTILSGVVTCFRLICGLLISKVVAIYTGPIGIANLGQLQNFVAFINGFISSQVSQGVNRYSAENQGDYENACFYWRASIKLSFIACCFIISTGVLFSSKISEYLFYQSDLYWLIIIALCALPLNIVNNIFLGVLNGLNEYNRFFLANVLAISSSLLSMVGLVYFFGLKGALVSASLNNAVAGVWLITIIIKRPWFKFKYWVGHTPRHNITQMKNYFYMGVIGALTGPISMIVVRTILTNNFSLEDAGYWQAVNRISEAYLAVLTTALTVYYFPKTAAARRYSEYITLLKTGACIVVPLALSMALTIYGLKDFIISILFTADFIRARELFLFQNIGDFLRICSWLFATILLAKGYFKINAILEVMFSIIFPVLVKFLIINFGLNAVSIAYSITYALYFIVVCIIYCWHLTKKA

SEQ ID NO: 90 - DNA, 1266 bp, Escherichia coli
atggtattaacagtgaaaaaaattttagcgtttggctattctaaagtactaccaccggttattgaacagtttgtcaatccaatttgcatcttcattatcacaccactaatactcaaccacctgggtaagcaaagctatggtaattggattttattaattactattgtatctttttctcagttaatatgtggaggatgttccgcatggattgcaaaaatcattgcagaacagagaattcttagtgatttatcaaaaaaaaatgctttacgtcaaatttcctataatttttcaattgttattatcgcatttgcggtattgatttcttttcttatattaagtatttgtttcttcgatgttgcgaggaataattcttcattcttattcgcgattattatttgtggtttttttcaggaagttgataatttatttagtggtgcgctaaaaggttttgaaaaatttaatgtatcatgtttttttgaagtaattacaagagtgctctgggcttctatagtaatatatggcatttacggaaatgcactcttatattttacatgtttagcctttaccattaaaggtatgctaaaatatattcttgtatgtctgaatattaccggttgtttcatcaatcctaattttaatagagttgggattgttaatttgttaaatgagtcaaaatggatgtttcttcaattaactggtggcgtctcacttagtttgtttgataggctcgtaataccattgattttatctgtcagtaaactggcttcttatgtcccttgccttcaactagctcaattgatgttcactctttctgcgtctgcaaatcaaatattactaccaatgtttgctagaatgaaagcatctaacacatttccctctaattgtttttttaaaattctgcttgtatcactaatttctgttttgccttgtcttgcgttattcttttttggtcgtgatatattatcaatatggataaaccctacatttgcaactgaaaattataaattaatgcaaattttagctataagttacattttattgtcaatgatgacatcttttcatttcttgttattaggaattggtaaatctaagcttgttgcaaatttaaatctggttgcagggctcgcacttgctgcttcaacgttaatcgcagctcattatggcctttatgcaatatctatggtaaaaataatatatccggcttttcaattttattacctttatgtagcttttgtctattttaatagagcgaaaaatgtctattga

SEQ ID NO: 91 - protein, 420 aa, Escherichia coli
MVLTVKKILAFGYSKVLPPVIEQFVNPICIFIITPLILNHLGKQSYGNWILLITIVSFSQLICGGCSAWIAKIIAEQRILSDLSKKNALRQISYNFSIVIIAFAVLISFLILSICFFDVARNNSSFLFAIIICGFFQEVDNLFSGALKGFEKFNVSCFFEVITRVLWASIVIYGIYGNALLYFTCLAFTIKGMLKYILVCLNITGCFINPNFNRVGIVNLLNESKWMFLQLTGGVSLSLFDRLVIPLILSVSKLASYVPCLQLAQLMFTLSASANQILLPMFARMKASNTFPSNCFFKILLVSLISVLPCLALFFFGRDILSIWINPTFATENYKLMQILAISYILLSMMTSFHFLLLGIGKSKLVANLNLVAGLALAASTLIAAHYGLYAISMVKIIYPAFQFYYLYVAFVYFNRAKNV

SEQ ID NO: 92 - DNA, 1395 bp, Escherichia coli
atgggagcagagtcatatggattaattggcttttttactatgcttcaggcgctgtttggtctcttagacttagggctaactccaacaattggtcgtgaaacagctcgctatcatggcgggacaatgacagtgctggactacagaaaattgctaagaactcttcatattttgttcttcactatcgctactattggtggagtatttctatttttcttagcacccatcatttcggttcattggttaaaggttacaacaatatcaattgacaccgtaaattactgtgtaaagataatggcattctctatagcattacgatggataggtgggttatataggggaatcattagcggtagcgaaaggttagactggctaagctatttaaatatttttataacaacgcttagattcatcattatatttcctgtcatgaaagtaaaaggttatactcctttcgtatttttcacatatcaattaatcgttgcgatcattgagtatttgtctttattatgtttgtctactttattaactcctaaattgaaatcagcaaacgagaaaattggtttttcattttcgcctattatgtcactgatgcgcttttccttaacagttgcttttacttcctctatttggatattgattactcagagtgataaactcatcttgtcgggtatattggaattaagtgaatacgggcatttcacactttctgtgttgttggctagtggcattctgatgattaatattccggttacaaactctatattaccgaggcttgcgcgtttacatgctgaaaataagcatgatgaacttctccgtatttataaaaatacgactatgttagtatgcatcttgggagtaagcgcatcgctagttgttgctatctatgcagaaccattgcttcttatctggactggtgatgcagaagtttcagcaagtgcatcgcctattctctcattgtatgccattggaaatggtttactggcagtatcagctttcccatattatctacaatatgctcaagggcggctaaagtatcacttctggggaaatattgtaatgttcttcatgctaatccccacaattattgtacttgctcgtaattttggtggagttggcgcgggttatgcctggttaatagttaatgtcttttatctttttgtatggactgcattggttcatcataaattaatacccggattacatctctcttggttaatgagtatcggtatgataacaattcctaccggtattattgtattttttctctcctttattgtccgatttagcggtaatagattgcatgacataattgtgctgggcttaatatcaattttggctctgatggtttcaatagtatcatgctggctaatcaaaagaatgaatctcggggtgtaa

SEQ ID NO: 93 - protein, 464 aa, Escherichia coli
MGAESYGLIGFFTMLQALFGLLDLGLTPTIGRETARYHGGTMTVLDYRKLLRTLHILFFTIATIGGVFLFFLAPIISVHWLKVTTISIDTVNYCVKIMAFSIALRWIGGLYRGIISGSERLDWLSYLNIFITTLRFIIIFPVMKVKGYTPFVFFTYQLIVAIIEYLSLLCLSTLLTPKLKSANEKIGFSFSPIMSLMRFSLTVAFTSSIWILITQSDKLILSGILELSEYGHFTLSVLLASGILMINIPVTNSILPRLARLHAENKHDELLRIYKNTTMLVCILGVSASLVVAIYAEPLLLIWTGDAEVSASASPILSLYAIGNGLLAVSAFPYYLQYAQGRLKYHFWGNIVMFFMLIPTIIVLARNFGGVGAGYAWLIVNVFYLFVWTALVHHKLIPGLHLSWLMSIGMITIPTGIIVFFLSFIVRFSGNRLHDIIVLGLISILALMVSIVSCWLIKRMNLGV

SEQ ID NO: 94 - DNA, 1239 bp, Escherichia coli
atgtttaatactatgcttaagtattactcaagtgttggattaagagggattactctacttactaaatttattttcattgttttgcttgctcgacttttaccatcaacagatttaggagtgtatggattaattaatgcagctgtaggatatggtattttcgttgtaggttttgagttttatacgtattcaacgagagaaataattaactcgcaaaaaaataggcttttttttatactaaaaaatcaagctctatttactgttatatcttatatactatgtataccggcatttatttttttattatatttagaaatattaccatctggaagtgaatactggtttatcctacttttattttttgagcacttatcacaagagattaatagagttctaataacaatagaaagtcaatcgattgcaagttttattctttttgtaagacaaggtgtatggtgttggttagctatagctgtgatgctagtgtatccgaacttaagaaatataacagttgtatttattttttggtttggtggtactgtgtccgcgagtgtgcttggagtggcttatattttaaataaaaaaaaacaaagcgatattacaaactgggattggacgtggataaaaaaaggtataaagctgtctgtaccaatgctaattgcagcccttgcactacgaggctttttcacgtttgatagattcgcggtagaaaaaatatcgggcctagaagttttgggaggatatacattatttgttagtatgacttcagctattcaatcatttttggatactattttgatatctttttcatttccaaagcttgccttgttatattcagggaaaaaatatataaaatttaaatctgagttaagaaaattcacttataaattaattttactactatctttcttgagcatctgttgcttttttactgggattattttggttaagtggttggataaacgagattacatacaattatttcctgtatttatattattaatagcagcgacttatatctattgtataagtcttattccacatattgctttatacgcgatgagagaagatcgttacatattagtaagtcaactgatatcatttttatcttttttactatttgttttttttagcgtatatcaaagtgatatctattacttgctaattggtatgatagctagttttgtattacttttgatcttaaaaatgatcccgttatataaaattctaaaaaaggtttaa

SEQ ID NO: 95 - protein, 412 aa, Escherichia coli
MFNTMLKYYSSVGLRGITLLTKFIFIVLLARLLPSTDLGVYGLINAAVGYGIFVVGFEFYTYSTREIINSQKNRLFFILKNQALFTVISYILCIPAFIFLLYLEILPSGSEYWFILLLFFEHLSQEINRVLITIESQSIASFILFVRQGVWCWLAIAVMLVYPNLRNITVVFIFWFGGTVSASVLGVAYILNKKKQSDITNWDWTWIKKGIKLSVPMLIAALALRGFFTFDRFAVEKISGLEVLGGYTLFVSMTSAIQSFLDTILISFSFPKLALLYSGKKYIKFKSELRKFTYKLILLLSFLSICCFFTGIILVKWLDKRDYIQLFPVFILLIAATYIYCISLIPHIALYAMREDRYILVSQLISFLSFLLFVFFSVYQSDIYYLLIGMIASFVLLLILKMIPLYKILKKV

SEQ ID NO: 96 - DNA, 1239 bp, Escherichia coli
atgaaaataataatttttagagtgctaacttttttctttgttatcttttctgttaatgtggttgcgaaggaatttaccttagatttctcgacagcaaagacgtatgtagattcgctgaatgtcattcgctctgcaataggtactccattacagactatttcatcaggaggtacgtctttactgatgattgatagtggcacaggggataatttgtttgcagttgatgtcagagggatagatccagaggaagggcggtttaataatctacggcttattgttgaacgaaataatttatatgtgacaggatttgttaacaggacaaataatgttttttatcgctttgctgatttttcacatgttacctttcctggtacaactgcggttacattgtctggtgacagtagctataccacgttacagcgtgttgcggggatcagtcgtacggggatgcagataaatcgccattcgttgactacttcttatctggatttaatgtcgcatagcggaacctcactgacgcagtctgtggcaagagcgatgttacggtttgttactgtgacagctgaagctttacgttttcggcaaattcagaggggatttcgtacaacacttgatgatctcagtgggcgttcttatgtaatgactgctgaagatgttgatcttacgttgaactggggaaggttgagtagtgtcctgcctgactatcatggacaagactctgttcgtgttggaagaatttcttttggaagtgttaatgcaattctgggtagcgtggcattaatactgaattgtcatcatcatgcatcgcgagttgccagaattgtacctaatgagtttccttctatgtgcccggtagatggaagagtgcgtgggattacgcacaataaaatattgtgggactcatccactctgggggcaattttgatacgcagggctattagcagttgaggggggtaaaatgaaaaaaatattattaatagctgcatcactttcatttttttcagcaagtgtgctggctgcgccagattgtgtaactgggaaggtggagtatacaaaatataatgatgacgatacctttacagttaaagtgggagataaagaattatttactaacagatggaatcttcagtctcttcttctcagtgcacaaattacggggatgacggtaaccattaaaactaatgcctgtcataatggagggggattcagcgaggttattttccgttgactcagaatagc

SEQ ID NO: 97 - protein, 315 aa, Escherichia coli
MKIIIFRVLTFFFVIFSVNVVAKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVARIVPNEFPSMCPVDGRVRGITHNKILWDSSTLGAILIRRAISS

SEQ ID NO: 98 - protein, 89 aa, Escherichia coli
MKKILLIAASLSFFSASVLAAPDCVTGKVEYTKYNDDDTFTVKVGDKELFTNRWNLQSLLLSAQITGMTVTIKTNACHNGGGFSEVIFR

SEQ ID NO: 99 - DNA, 1228 bp, Escherichia coli
atgaaaataataatttttagagtgctaacttttttctttgttatcttttctgttaatgtggttgcgaaggaatttaccttagatttctcgacagcaaagacgtatgtagattcgctgaatgtcattcgctctgcaataggtactccattacagactatttcatcaggaggtacgtctttactgatgattgatagtggcacaggggataatttgtttgcagttgatgtcagagggatagatccagaggaagggcggtttaataatctacggcttattgttgaacgaaataatttatatgtgacaggatttgttaacaggacaaataatgttttttatcgctttgctgatttttcacatgttacctttcctggtacaactgcggttacattgtctggtgacagtagctataccacgttacagcgtgttgcggggatcagtcgtacggggatgcagataaatcgccattcgttgactacttcttatctggatttaatgtcgcatagcggaacctcactgacgcagtctgtggcaagagcgatgttacggtttgttactgtgacagctgaagctttacgttttcggcaaattcagaggggatttcgtacaacacttgatgatctcagtgggcgttcttatgtaatgactgctgaagatgttgatcttacgttgaactggggaaggttgagtagtgtcctgcctgactatcatggacaagactctgttcgtgttggaagaatttcttttggaagtgttaatgcaattctgggtagcgtggcattaatactgaattgtcatcatcatgcatcgcgagttgccagaattgtacctaatgagtttccttctatgtgcccggtagatggaagagtgcgtgggattacgcacaataaaatattgtgggactcatccactctgggggcaattttgatacgcagggctattagcagttgaggggggtaaaatgaaaaaaatattattaatagctgcatcactttcatttttttcagcaagtgtgctggctgcgccagattgtgtaactgggaaggtggagtatacaaaatataatgatgacgatacctttacagttaaagtgggagataaagaattatttactaacagatggaatcttcagtctcttcttctcagtgcacaaattactgggatgacggtaaccattaaaactaatgcctgtcataatggagggggattcagcgaggttattttccgttga

SEQ ID NO: 100 - protein, 315 aa, Escherichia coli; identical to SEQ ID NO: 97

SEQ ID NO: 101 - protein, 89 aa, Escherichia coli; identical to SEQ ID NO: 98

SEQ ID NO: 102 - DNA, 1227 bp, Escherichia coli
atgaaaataatgatttttagggcgctaacatttttcttcgttatcttttcagttaatgcgattgctaaggagtttaccttagatttctcaacagcaaagaagtatgttgattcgctgaatgtcattcgctctgcaataggtacgccattacagactatttcatcgggaggtacatctttactgatgattgatagtggcacaggcgataatttatttgcagtcgatatcatggggttagaaccagaggaagagcggtttaacaatctacgactgattgttgaacgaaataatttatatgtgacaggatttgttaacaggacaaacaatgttttttatcgctttgctgatttttcacacgttacctttcctggtacaagagcggttacattgtctggtgacagtagctataccacgttacagcgtgttgcggggatcagtcgtacggggatgcagataaatcgccattcgctgactacttcttatctggatttgatgtcgtatagcggaacctcactgacgcagtctgtagcaagagcaatgttacggtttgttactgtgacagctgaagctttgcgttttcgccaaatccagaggggatttcgtacaacacttgatgatctcagtggacgttcttatgtaatgactgctgaagatgttgatcttacattgaattggggaagactgagtagtattctgcccgactatcatggacaagactccgttcgtgttggaagaatttcttttgggagtattaatgcaattctgggaagcgtggcattaatattaaattgccatcatcatgcatcacgagttgccagaatgacacctgatgagtttccttctatgtgcccgacagatggaagtgggcgtgggattactcacaataaaatattgtgggactcatctacactgggggcaattttgattcgcagaactatcagtagttgagggggtaaaatgaaaaaagtattattaatagctgtttcactttcatttctttcagcaagtgttctggcagcgccagattgtgtaaccggaaaggtggagtatacaaaatataatgatgacgatacttttacagttaaagtggcagataaagaattatttactaacagatggaatcttcagtctcttcttctaagtgcacaaattacggggatgacggtaactattaagaccactgcttgtcataatggagggggattcagcgaggttatttttcgttga

SEQ ID NO: 103 - protein, 315 aa, Escherichia coli
MKIMIFRALTFFFVIFSVNAIAKEFTLDFSTAKKYVDSLNVIRSAIGTPLQTISSGGTSLLMIDSGTGDNLFAVDIMGLEPEEERFNNLRLIVERNNLYVTGFVNRTNNVFYRFADFSHVTFPGTRAVTLSGDSSYTTLQRVAGISRTGMQINRHSLTTSYLDLMSYSGTSLTQSVARAMLRFVTVTAEALRFRQIQRGFRTTLDDLSGRSYVMTAEDVDLTLNWGRLSSILPDYHGQDSVRVGRISFGSINAILGSVALILNCHHHASRVARMTPDEFPSMCPTDGSGRGITHNKILWDSSTLGAILIRRTISS

SEQ ID NO: 104 - protein, 89 aa, Escherichia coli
MKKVLLIAVSLSFLSASVLAAPDCVTGKVEYTKYNDDDTFTVKVADKELFTNRWNLQSLLLSAQITGMTVTIKTTACHNGGGFSEVIFR

SEQ ID NO: 105 - DNA, 1274 bp, Escherichia coli
tacttcagccaaaaggaacaccttgtatatgaagcgtatattatttaaatgggtactgtgcctgttactgggcttttcttcggtatcctattcccgggaatttacgatagacttttcgactcaacaaagttatgtatcttcgttaaatagtatacggacagagatatcgacccctcttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacgccgggagacgtggacctcactctgaactgggggcgaatcagcaatgtgcttccggagtatcggggagaggatggtgtcagagtggggagaatatcctttaataatatatcggcgatactgggcactgtggccgttatactgaattgtcatcatcagggggcgcgttctgttcgcgccgtgaatgaagatagtcaaccagaatgtcagataactggcgacaggcccgttataaaaataaacaatacattatgggaaagtaatacagctgcagcgtttctgaacagaaagtcacagtttttatatacaacgggtaaataaaggagttaagtatgaagaagatgtttatggcggttttatttgcattagtttctgttaatgcaatggcggcggattgcgctaaaggtaaaattgagttttccaagtataatgagaatgatacattcacagtaaaagtggccggaaaagagtactggaccagtcgctggaatctgcaaccgttactgcaaagtgctcagttgacaggaatgactgtcacaatcaaatccagtacctgtgaatcaggctccggatttgctgaagtgcagtttaataatgactgaggcat

SEQ ID NO: 106 - protein, 319 aa, Escherichia coli
MKRILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEDSQPECQITGDRPVIKINNTLWESNTAAAFLNRKSQFLYTTGK

SEQ ID NO: 107 - protein, 89 aa, Escherichia coli
MKKMFMAVLFALVSVNAMAADCAKGKIEFSKYNENDTFTVKVAGKEYWTSRWNLQPLLQSAQLTGMTVTIKSSTCESGSGFAEVQFNND

SEQ ID NO: 108 - DNA, 1213 bp, Escherichia coli
tacttcagccaaaaggaacaccttgtatatgaagtgtatattatttaaatgggtactgtgcctgttactgggcttttcttcggtatcctattcccgggaatttacgatagacttttcgactcaacaaagttatgtatcttcgttaaatagtatacggacagagatatcgacccctcttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacgccgggagacgtggacctcactctgaactgggggcgaatcagcaatgtgcttccggagtatcggggagaggatggtgtcagagtggggagaatatcctttaataatatatcggcgatactgggcactgtggccgttatactgaattgtcatcatcagggggcgcgttctgttcgcgccgtgaatgaagatagtcaaccagaatgtcagataactggcgacaggcccgttataaaaataaacaatacattatgggaaagtaatacagctgcagcgtttctgaacagaaagtcacagtttttatatacaacgggtaaataaaggagttaagtatgaagaagatgtttatggcggttttatttgcattagtttctgttaatgcaatggcggcggattgcgctaaaggtaaaattgagttttccaagtataatgagaatgatacattcacagtaaaagtggccggaaaagagtactggaccagtcgctggaatctgcaaccgttactgcaaagtgctcagttgacaggaatgactgtcacaatcaaat

SEQ ID NO: 109 - protein, 319 aa, Escherichia coli
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEDSQPECQITGDRPVIKINNTLWESNTAAAFLNRKSQFLYTTGK

SEQ ID NO: 110 - protein, 108 aa, Escherichia coli
MTKNTRFSPEVRQRAIRMVLESQDEYDSQWAAICSIAPKIGCTPETLRVWVRQHERDTGGGDGGLTSAERQRLKELERENRELRRSNDILRQASAYFAKAEFDRLWKK

SEQ ID NO: 111 - DNA, 1260 bp, Escherichia coli
tacttcagccaaaaggaacacctgtatatgaagtgtatattatttaaatgggtactgtgcctgttactgggtttttcttcggtatcctattcccgggagtttatgatagacttttcgacccaacaaagttatgtctcttcgttaaatagtatacggacagagatatcgacccctcttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggctgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacaccggaagaagtggacctcacactgaactgggggagaatcagcaatgtgcttccggagtttcggggagagggtggtgtcagagtggggcgaatatcctttaataatatatcagcgatactgggcacagtggcggttatactgaattgccatcatcagggggcgcgttccgttcgcgccgtgaatgaagagatacaaccagaatgtcagataactggcgacaggccagttataaggataaacaatactttatgggaaagtaataccgcagctgcttttctgaatcgcagggcccactctttaaatacatccggagaataacaggagttaaatatgaagaagatatttgtagcggctttatttgcttttgtttctgttaatgcaatggcagctgattgtgcaaaaggtaaaattgagttctctaagtataatgagaatgatacattcacagtaaaagtggccgggaaagagtactggactaaccgctggaatctgcaaccgctactgcaaagcgcacagttaacaggaatgacggtaacaatcaaatcaaatacctgtgcgtcaggttcaggatttgctgaagtgcagtttaat

SEQ ID NO: 112 - protein, 319 aa, Escherichia coli
MKCILFKWVLCLLLGFSSVSYSREFMIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPEEVDLTLNWGRISNVLPEFRGEGGVRVGRISFNNISAILGTVAVILNCHHQGARSVRAVNEEIQPECQITGDRPVIRINNTLWESNTAAAFLNRRAHSLNTSGE

SEQ ID NO: 113 - protein, 87 aa, Escherichia coli
MKKIFVAALFAFVSVNAMAADCAKGKIEFSKYNENDTFTVKVAGKEYWTNRWNLQPLLQSAQLTGMTVTIKSNTCASGSGFAEVQFN

SEQ ID NO: 114 - DNA, 1232 bp, Escherichia coli
atgaagtgtatattgttaaagtggatactgtgtctgttactgggtttttcttcggtatcctattcccaggagtttacgatagacttttcgactcaacaaagttatgtatcttcgttaaatagtatacggacagcgatatcgacccctcttgaacatatatctcagggagctacatcggtatccgttattaatcatacaccaccaggaagttatatttccgtaggtatacgagggcttgatgtttatcaggagcgttttgaccatcttcgtctgattattgaacgaaataatttatatgtggctggatttgttaatacgacaacaaatactttctacagattttcagattttgcacatatatcattgcccggtgtgacaactatttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatcaagagcagttctgcgttttgtcactgtcacagcagaagccttacggttcaggcaaatacagagagaatttcgtctggcactgtctgaaactgctcctgtttatacgatgacgccggaagacgtggacctcactctgaactgggggagaatcagcaatgtgcttccggagtatcggggagaggctggtgtcagagtggggagaatatcctttaataatatatcagcgatacttggtactgtggccgttatactgaattgccatcatcagggcgcgcgttctgttcgcgccgtgaatgaagagagtcaaccagaatgtcagataactggcgacaggcccgttataaaaataaacaatacattatgggaaagtaatacagcagcagcgtttctgaacagaaagtcacagtctttatatacaactggtgaatgaaaggagttaagaatgaagaagatgtttatagcggttttatttgcattggtttctgttaatgcaatggcggcggattgtgctaaaggtaaaattgagttttccaagtataatgaggataatacctttactgtgaaggtgtcaggaagagaatactggacgaacagatggaatttgcagccattgttacaaagtgctcagctgacagggatgactgtaacaatcatatctaatacctgcagttcaggctcaggctttgcccaggtgaagtttaa

SEQ ID NO: 115 - protein, 319 aa, Escherichia coli
MKCILLKWILCLLLGFSSVSYSQEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVRAVNEESQPECQITGDRPVIKINNTLWESNTAAAFLNRKSQSLYTTGE

SEQ ID NO: 116 - protein, 87 aa, Escherichia coli
MKKMFIAVLFALVSVNAMAADCAKGKIEFSKYNEDNTFTVKVSGREYWTNRWNLQPLLQSAQLTGMTVTIISNTCSSGSGFAQVKFN

SEQ ID NO: 117 - DNA, 1189 bp, Escherichia coli
gcttgtcttcagcatcttatgcagatgagtttactgtggatttctcttcgcaaaagagctatgttgattcattgaatagtataaggtcggcaatatccactccacttggaaatatatctcagggtggtgtttctgtttcagtaattaatcatgttccaggcggaaactatatatcattgaatgttagaggccttgatccatatagcgagagatttaaccacctccgtttaataatggaacggaataacttatatgttgcaggctttattaatactgaaacgaataccttttacagattctccgatttctcacatatttcagtgcctgatgtgataactgtttccatgacgacggacagcagttattcatcattacagcgaatcgcagatctggaacgtacagggatgcagattgggcgtcattcactggttggttcatatctggatttaatggagttcagaggacgttccatgacccgcgcatcatccagagctatgctgcgttttgtcacagtgatagcagaagctctgcgattcagacaaatacagcggggattccgaccggcgctgtctgaggcatctccgctttatacaatgacggctcaggatgttgaccttaccctgaactggggaagaataagtaatgttcttccagagtacagaggagaggaaggggtaagaatcggtaggatatcttttaatagtctttctgcgattctcggaagtgttgcggtcatccttaattgccactcaaccggaagttattcagttcgttccgtgagccaaaaacagaaaacagaatgccagattgttggagacagggcggccattaaagtaaataatgttttgtgggaagcgaatacaatcgctgctttattaaatcgcaagcctcaggatcttactgaaccaaaccaataacagggggtgaatatgaagaagatgattattgcagttttattcggtctcttttctgctaattccatggcggcggattgtgctgtaggaaaaattgagttttccaagtataatgaggatgatacctttactgtgaaggtgtcaggaagagaatactggacgaacagatggaatttgcagccattgttacaaagtgctcagctgacagggatgactgtaacaatcatatctaatacctgcagttcaggctcaggctttgcccaggtgaagtttaa

SEQ ID NO: 118 - protein, 319 aa, Escherichia coli
MRHILLKLVLFFCVCLSSASYADEFTVDFSSQKSYVDSLNSIRSAISTPLGNISQGGVSVSVINHVPGGNYISLNVRGLDPYSERFNHLRLIMERNNLYVAGFINTETNTFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 119 - protein, 87 aa, Escherichia coli
MKKMIIAVLFGLFSANSMAADCAVGKIEFSKYNEDDTFTVKVSGREYWTNRWNLQPLLQSAQLTGMTVTIISNTCSSGSGFAQVKFN

SEQ ID NO: 120 - DNA, 1269 bp, Escherichia coli
tacttcagccaaaaggaatacctgtatatgaagtgtatattatttaaatgggtactgtgcctgttactgggcttttcttcggtatcctattcccgggaatttacgatagacttttcgactcaacaaagttatgtatcttcgttaaatagtatacggacagaaatatcgacccctcttgagcatatatctcagggggctacatcggtatctgttattaatcatactccaccgggtagttatatttctgtggatatccgagggcttgatatctatgaggcgcgttttgaccatcttcgtctgattattgagcaaaataatttatatgtggccgggttcgttaatacggccacaaatactttctaccgtttttcagattttacacatatatcagtgcccggtgtgacaactgtttccatgacaacggacagcagttataccacgctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgttacagcagaagccttacggttcaggcaaatacagagagaatttcgtctggcactgtctgaaactgctcctgtttatacgatgacgccggaagacgtggacctcactctgaactgggggagaatcagcaatgtgcttccggagtatcggggagaggatagtgtcagagtggggagaatatcctttaataatatatcagcaatactgggtactgtagcggttattctgaattgccatcatcaggggacgcgttcggttcgctacgtgaatgaagagatgcaaccagaatgtcagataagtggcgacaggccagttataaaaataaacaatacattatgggaaagtaatacagcagcagcctttctgaacagaaagtctcagtcattatatacaacgggtgaatgaaaggagttaagcatgaagaagatgtttatggcggttttatttgcattggtttctgttaatgcaatggcggcggattgtgctaaaggtaaaattgagttttccaaatataatggggataacacatttactgtaaaggttgacgggaaagaatactggactaaccggtggaatttgcagccgttgttacaaagtgcacagttaacaggaatgaccgtaacaatcaaatccaatacctgtgaatcaggctctggatttgctgaagtgcagtttaataatgactga

SEQ ID NO: 121 - protein, 319 aa, Escherichia coli
MKCILFKWVLCLLLGFSSVSYSREFTIDFSTQQSYVSSLNSIRTEISTPLEHISQGATSVSVINHTPPGSYISVDIRGLDIYEARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEDSVRVGRISFNNISAILGTVAVILNCHHQGTRSVRYVNEEMQPECQISGDRPVIKINNTLWESNTAAAFLNRKSQSLYTTGE

SEQ ID NO: 122 - protein, 89 aa, Escherichia coli
MKKMFMAVLFALVSVNAMAADCAKGKIEFSKYNGDNTFTVKVDGKEYWTNRWNLQPLLQSAQLTGMTVTIKSNTCESGSGFAEVQFNND

SEQ ID NO: 123 - DNA, 20 bp, Artificial Sequence
  Synthetic Construct
ccagtctgcgtctgattcca

SEQ ID NO: 124 - DNA, 22 bp, Artificial Sequence
  Synthetic Construct
cgttgcgctcattacttctgaa

SEQ ID NO: 125 - DNA, 26 bp, Artificial Sequence
  Synthetic Construct
cattgatcaggatttttctggtgata

SEQ ID NO: 126 - DNA, 21 bp, Artificial Sequence
  Synthetic Construct
ctcatgcggaaatagccgtta

SEQ ID NO: 127 - DNA, 30 bp, Artificial Sequence
  Synthetic Construct
atagtctcgccagtattcgccaccaatacc

SEQ ID NO: 128 - DNA, 21 bp, Artificial Sequence
  Synthetic Construct
gtgtcagtagggaagcgaaca

SEQ ID NO: 129 - DNA, 19 bp, Artificial Sequence
  Synthetic Construct
atcatgttttccgccaatg

SEQ ID NO: 130 - DNA, 24 bp, Artificial Sequence
  Synthetic Construct
tctgttgaagagctcattggcgga

SEQ ID NO: 131 - DNA, 24 bp, Artificial Sequence
  Synthetic Construct
tatcagcaccaaagagcgggaaca

SEQ ID NO: 132 - DNA, 24 bp, Artificial Sequence
  Synthetic Construct
cccttatgaagagccagtactgaa

SEQ ID NO: 133 - DNA, 26 bp, Artificial Sequence
  Synthetic Construct
aaaggcgtcgtttcagccagccggaa

SEQ ID NO: 134 - DNA, 24 bp, Artificial Sequence
  Synthetic Construct
ttcctttgccatggcggagaattg

SEQ ID NO: 135 - DNA, 24 bp, Artificial Sequence
  Synthetic Construct
agcggctcctgtctgattaacgat

SEQ ID NO: 136 - DNA, 24 bp, Artificial Sequence
  Synthetic Construct
acgctggaatggtctggagattgt

SEQ ID NO: 137 - DNA, 24 bp, Artificial Sequence
  Synthetic Construct
atccaccaccggatttctctggtt

SEQ ID NO: 138 - DNA, 20 bp, Artificial Sequence
  Synthetic Construct
tttcgctcacaacaatcgaa

SEQ ID NO: 139 - DNA, 21 bp, Artificial Sequence
  Synthetic Construct
ttggccaaaagaaagtgtagc

SEQ ID NO: 140 - DNA, 26 bp, Artificial Sequence
  Synthetic Construct
attgtaactgatgttatttcgtttgg

SEQ ID NO: 141 - DNA, 23 bp, Artificial Sequence
  Synthetic Construct
grcatcaaaagcgaaatcacacc

SEQ ID NO: 142 - DNA, 20 bp, Artificial Sequence
  Synthetic Construct
accatgaatgcgtgctgtaa

SEQ ID NO: 143 - DNA, 20 bp, Artificial Sequence
  Synthetic Construct
ctggacggactggatttgtt

SEQ ID NO: 144 - DNA, 23 bp, Artificial Sequence
  Synthetic Construct
catgttgaaggctggaastttgt

SEQ ID NO: 145 - DNA, 20 bp, Artificial Sequence
  Synthetic Construct
ccgctacagggcgatatgtt

SEQ ID NO: 146 - DNA, 20 bp, Artificial Sequence
  Synthetic Construct
tttcaggaacggtgagatcc

SEQ ID NO: 147 - DNA, 20 bp, Artificial Sequence
  Synthetic Construct
ccctttactccgggaagaac

SEQ ID NO: 148 - DNA, 20 bp, Artificial Sequence
  Synthetic Construct
ggccgcttttcagttatgag

SEQ ID NO: 149 - DNA, 20 bp, Artificial Sequence
  Synthetic Construct
cgaccggagccactttagtt

SEQ ID NO: 150 - DNA, 20 bp, Artificial Sequence
  Synthetic Construct
gagagcagcactttcgcttt

SEQ ID NO: 151 - DNA, 20 bp, Artificial Sequence
  Synthetic Construct
tggatacccgaacactcaca

SEQ ID NO: 152 - DNA, 29 bp, Artificial Sequence
  Synthetic Construct
tttgtyactgtsacagcwgaagcyttacg

SEQ ID NO: 153 - DNA, 26 bp, Artificial Sequence
  Synthetic Construct
ccccagttcarwgtragrtcmacdtc

SEQ ID NO: 154 - DNA, 31 bp, Artificial Sequence
  Synthetic Construct
ctggatgatctcagtgggcgttcttatgtaa

SEQ ID NO: 155 - DNA, 29 bp, Artificial Sequence
  Synthetic Construct
tttgtyactgtsacagcwgaagcyttacg

SEQ ID NO: 156 - DNA, 26 bp, Artificial Sequence
  Synthetic Construct
ccccagttcarwgtragrtcmacdtc

SEQ ID NO: 157 - DNA, 27 bp, Artificial Sequence
  Synthetic Construct
tcgtcaggcactgtctgaaactgctcc

SEQ ID NO: 158 - DNA, 23 bp, Artificial Sequence
  Synthetic Construct
ataaatcgccattcgttgactac

SEQ ID NO: 159 - DNA, 21 bp, Artificial Sequence
  Synthetic Construct
agaacgcccactgagatcatc

SEQ ID NO: 160 - DNA, 21 bp, Artificial Sequence
  Synthetic Construct
ggcactgtctgaaactgctcc

SEQ ID NO: 161 - DNA, 22 bp, Artificial Sequence
  Synthetic Construct
tcgccagttatctgacattctg

SEQ ID NO: 162 - DNA, 20 bp, Artificial Sequence
  Synthetic Construct
atctgcccactcatgctttc

SEQ ID NO: 163 - DNA, 20 bp, Artificial Sequence
  Synthetic Construct
ggccagcgattactttacca

SEQ ID NO: 164 - DNA, 20 bp, Artificial Sequence
  Synthetic Construct
cggagaagtcaccacctgat

SEQ ID NO: 165 - DNA, 20 bp, Artificial Sequence
  Synthetic Construct
ttgatgatggttgcactggt

SEQ ID NO: 166 - DNA, 20 bp, Artificial Sequence
  Synthetic Construct
ctgctccgttgttgggtaac

SEQ ID NO: 167 - DNA, 20 bp, Artificial Sequence
  Synthetic Construct
gcatcagcgtggttttacct

SEQ ID NO: 168 - DNA, 20 bp, Artificial Sequence
  Synthetic Construct
ttggagcgttaactggacct

SEQ ID NO: 169 - DNA, 24 bp, Artificial Sequence
  Synthetic Construct
atattcgctatatcttcttgcggc

SEQ ID NO: 170 - DNA, 28 bp, Artificial Sequence
  Synthetic Construct
aggcttatctggctgttcttactacggc

SEQ ID NO: 171 - DNA, 21 bp, Artificial Sequence
  Synthetic Construct
tgttccaggtggtaggattcg

SEQ ID NO: 172 - DNA, 21 bp, Artificial Sequence
  Synthetic Construct
tcacgatgttgatcatctggg

SEQ ID NO: 173 - DNA, 29 bp, Artificial Sequence
  Synthetic Construct
tgaaggcgaggcaacacattatatagtgc

SEQ ID NO: 174 - DNA, 22 bp, Artificial Sequence
  Synthetic Construct
aggcgctgtttggtctcttaga

SEQ ID NO: 175 - DNA, 20 bp, Artificial Sequence
  Synthetic Construct
gaaccgaaatgatgggtgct

SEQ ID NO: 176 - DNA, 28 bp, Artificial Sequence
  Synthetic Construct
cgctatcatggcgggacaatgacagtgc

SEQ ID NO: 177 - DNA, 19 bp, Artificial Sequence
  Synthetic Construct
aaactgggattggacgtgg

SEQ ID NO: 178 - DNA, 19 bp, Artificial Sequence
  Synthetic Construct
cccaaaacttctaggcccg

SEQ ID NO: 179 - DNA, 29 bp, Artificial Sequence
  Synthetic Construct
tgctaattgcagcccttgcactacgaggc

SEQ ID NO: 180 - DNA, 22 bp, Artificial Sequence
  Synthetic Construct
gtatcgctgaaattagaagcgc

SEQ ID NO: 181 - DNA, 21 bp, Artificial Sequence
  Synthetic Construct
agttgaaacacccgtaatggc

SEQ ID NO: 182 - DNA, 28 bp, Artificial Sequence
  Synthetic Construct
tggttcggttggattgtccataagaggg

SEQ ID NO: 183 - DNA, 18 bp, Artificial Sequence
  Synthetic Construct
cgttgtgcatggtggcat

SEQ ID NO: 184 - DNA, 22 bp, Artificial Sequence
  Synthetic Construct
tggccaaaccaactatgaactg

SEQ ID NO: 185 - DNA, 26 bp, Artificial Sequence
  Synthetic Construct
attttttcgtcgaagtgggctgtaca

SEQ ID NO: 186 - DNA, 21 bp, Artificial Sequence
  Synthetic Construct
ctgaaaagagccagaacgtgc

SEQ ID NO: 187 - DNA, 22 bp, Artificial Sequence
  Synthetic Construct
tgcctaagatcattacccggac

SEQ ID NO: 188 - DNA, 20 bp, Artificial Sequence
  Synthetic Construct
tagcgggacaattgtcacgg

SEQ ID NO: 189 - DNA, 23 bp, Artificial Sequence
  Synthetic Construct
gtctttcggagaaacattctgcc

SEQ ID NO: 190 - DNA, 20 bp, Artificial Sequence
  Synthetic Construct
ctgcgacacggtatctgaaa

SEQ ID NO: 191 - DNA, 20 bp, Artificial Sequence
  Synthetic Construct
accgataaatgggaccaaca

SEQ ID NO: 192 - DNA, 20 bp, Artificial Sequence
  Synthetic Construct
cacgatgactggctgaagaa

SEQ ID NO: 193 - DNA, 20 bp, Artificial Sequence
  Synthetic Construct
cggtagtgcggaccttttta

SEQ ID NO: 194 - DNA, 20 bp, Artificial Sequence
  Synthetic Construct
atggcaggtctgctacaggt

SEQ ID NO: 195 - DNA, 20 bp, Artificial Sequence
  Synthetic Construct
tagcggaattttctgcatcc

SEQ ID NO: 196 - DNA, 20 bp, Artificial Sequence
  Synthetic Construct
atcattggcaacactggtga

SEQ ID NO: 197 - DNA, 20 bp, Artificial Sequence
  Synthetic Construct
aaagatgcctcaggagcaga

SEQ ID NO: 198 - DNA, 20 bp, Artificial Sequence
  Synthetic Construct
ttctttctcccgacatccag

SEQ ID NO: 199 - DNA, 20 bp, Artificial Sequence
  Synthetic Construct
tatgggcctgttctcctctg

SEQ ID NO: 200 - DNA, 20 bp, Artificial Sequence
  Synthetic Construct
tgtcagccagaaccactgac

SEQ ID NO: 201 - DNA, 20 bp, Artificial Sequence
  Synthetic Construct
gcctttttccttgtcatcca

SEQ ID NO: 202 - DNA, 20 bp, Artificial Sequence
  Synthetic Construct
tatgggcctgttctcctctg

SEQ ID NO: 203 - DNA, 20 bp, Artificial Sequence
  Synthetic Construct
ttctttctcccgacatccag

SEQ ID NO: 204 - DNA, 20 bp, Artificial Sequence
  Synthetic Construct
caacctggacaggaggtcat

SEQ ID NO: 205 - DNA, 20 bp, Artificial Sequence
  Synthetic Construct
gcaccccggtttttatttct

SEQ ID NO: 206 - DNA, 20 bp, Artificial Sequence
  Synthetic Construct
gtgcatgatgtatggcaagc

SEQ ID NO: 207 - DNA, 20 bp, Artificial Sequence
  Synthetic Construct
ggaacccgggactgtttaat

SEQ ID NO: 208 - DNA, 20 bp, Artificial Sequence
  Synthetic Construct
agtcaactatccgggggaag

SEQ ID NO: 209 - DNA, 20 bp, Artificial Sequence
  Synthetic Construct
ctgtgggatttccgtgattt

SEQ ID NO: 210 - DNA, 20 bp, Artificial Sequence
  Synthetic Construct
agagtgaaggggaacgaggt

SEQ ID NO: 211 - DNA, 20 bp, Artificial Sequence
  Synthetic Construct
tccggtaaccagaacctcac